(19) United States
(12) Patent Application Publication
(10) Pub. No.: US 2024/0409938 A1
(43) Pub. Date: Dec. 12, 2024

(54) LIPOPHILIC SIRNA CONJUGATES FOR THE TREATMENT OF CENTRAL NERVOUS SYSTEM DISEASES

(71) Applicant: Vanderbilt University, Nashville, TN (US)

(72) Inventors: Craig L. Duvall, Nashville, TN (US); Ethan S. Lippmann, Nashville, TN (US); Alexander G. Sorets, Nashville, TN (US)

(21) Appl. No.: 18/737,720

(22) Filed: Jun. 7, 2024

Related U.S. Application Data
(60) Provisional application No. 63/507,015, filed on Jun. 8, 2023.

(51) Int. Cl. C12N 15/113; A61K 47/10; A61K 47/20; A61K 47/26; A61K 47/42; A61K 47/54

(52) U.S. Cl. CPC C12N 15/113; A61K 47/10; A61K 47/20; A61K 47/26; A61K 47/42; A61K 47/542; C12N 2310/14; C12N 2310/315; C12N 2320/32

(57) ABSTRACT

Disclosed herein are methods of treating central nervous system diseases, such as Alzheimer's disease and Huntington's disease, using lipophilic siRNA conjugates that can bind to albumin. The disclosed methods can advantageously provide widespread, long-term gene silencing in the central nervous system. An example method includes administering to a subject an effective amount of a conjugate, optionally in combination with a pharmaceutically acceptable excipient, wherein the conjugate includes a siRNA capable of inhibiting expression of a protein associated with the central nervous system disease; a lipophilic ligand capable of binding albumin; and a linker attaching the siRNA to the lipophilic ligand, the linker including a branching molecule attached to the siRNA, and a hydrophilic spacer attaching the branching molecule to the lipophilic ligand.

### CROSS-REFERENCE TO RELATED APPLICATIONS

This application claims priority to U.S. Provisional Patent Application No. 63/507,015 filed on Jun. 8, 2023 which is incorporated fully herein by reference.

### STATEMENT REGARDING FEDERALLY SPONSORED RESEARCH

This invention was made with government support under grant number 1R21AG077807-01 awarded by the NIA. The government has certain rights in the invention.

### REFERENCE TO AN ELECTRONIC SEQUENCE LISTING

The contents of the electronic sequence listing (093386-0025-US02 Sequence Listing.xml; Size: 16,037; and Date of Creation: Jun. 7, 2024) is herein incorporated by reference in its entirety.

### TECHNICAL FIELD

The present disclosure relates to lipophilic siRNA conjugates and their use in the treatment of central nervous system diseases.

### INTRODUCTION

Improved efficacy of therapies can be useful in treating diseases, such as diseases related to the central nervous system.

### SUMMARY

In one aspect, disclosed are methods of treating a central nervous system (CNS) disease in a subject in need thereof, the method comprising administering to the subject an effective amount of a disclosed conjugate, optionally in combination with a pharmaceutically acceptable excipient, wherein the conjugate comprises a siRNA capable of inhibiting expression of a protein associated with the CNS disease; a lipophilic ligand capable of binding albumin; and a linker attaching the siRNA to the lipophilic ligand, the linker comprising a branching molecule attached to the siRNA, and a hydrophilic spacer attaching the branching molecule to the lipophilic ligand.

In another aspect, disclosed are methods of delivering a therapeutic to a CNS of a subject in need thereof, the method comprising administering a disclosed conjugate to the subject intravenously or intracerebroventricularly, wherein the conjugate localizes to the subject's CNS, and wherein the conjugate comprises a siRNA capable of inhibiting expression of a protein associated with the CNS disease; a lipophilic ligand capable of binding albumin; and a linker attaching the siRNA to the lipophilic ligand, the linker comprising a branching molecule attached to the siRNA, and a hydrophilic spacer attaching the branching molecule to the lipophilic ligand.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A: Since reactive microgliosis—where microglia proliferate and undergo morphological changes—is a common feature of brain inflammation, injury, and neurodegeneration (including AD), it was decided to use this phenotype as a readout for responses to siRNA-lipid conjugates. A bilateral ICV injection was carried out with 120 μg siRNA (60 μg per ventricle), and after 48 hours, brains were isolated and single-cell suspensions were generated using a Miltenyi Adult Brain Dissociation kit. Microglia were labeled with an anti-CD11b antibody and their abundance was quantified by flow cytometry. Microglia numbers were unchanged by free siRNA or L2-siRNA treatment, whereas microglia numbers after Chol-siRNA treatment were roughly doubled, indicating activation and proliferation. Each dot represents an individual biological replicate (i.e., a single mouse brain; N=3-6 brains analyzed per condition). Bar graphs represent mean±SD. Statistical significance was calculated using a one-way ANOVA with Tukey's post-hoc test.

FIG. 1B: Representative images of brain cortices were acquired using confocal microscopy from mice receiving Chol-siRNA versus L2-siRNA. Fibrinogen is stained using an antibody to highlight deposition around vessels (staining positive with a CD31+ antibody), which is an indicator of focal BBB disruption and a contributor to neuroinflammation. This accumulation of fibrinogen was observed in brains of mice injected with Chol-siRNA but not L2-siRNA. Collectively, these results highlight an improved safety profile for L2-siRNA that is more favorable for clinical translation.

FIG. 2A: Nucleic acid structures are synthesized using standard zipper siRNA chemistry, characterized by alternating 2′ Omethyl (OMe) and fluoro bases, terminating with two phosphorothioate bonds. Sequences utilize the 5′ vinyl phosphonate (VP) on the anti-sense strand, and when applicable the Cy5 fluorophore is positioned at the 3′ position of the sense strand. Lipid conjugates are attached to the 5′ end of the sense strand.

FIG. 2B: L2-siRNA is composed of a divalent spacer and lipid tail. L2-siRNA may also be referred to herein as EG18. The spacer contains three repeats of hexaethylene glycol groups on each side of a brancher (totaling 18 ethylene glycol units) and the lipid is an 18-carbon alkyl chain. Chol-siRNA contains a triethylene glycol spacer separating the cholesterol moiety from the siRNA.

FIG. 2C: Overview of CSF anatomy highlighting interfaces for drug delivery. To examine biodistribution, Cy5-tagged compounds are injected into CSF through the intracerebroventricular route. CSF flows from the ventricular system to the subarachnoid space and then some CSF moves alongside vessels in the perivascular spaces of the brain.

FIG. 2D: Histological examination of CNS distribution either 2 or 48 hours after ICV injection of unconjugated siRNA, Chol-siRNA, or L2-siRNA (7.5-10 nmol dose) (left side panels). Cy5 signal is presented in red and DAPI nuclear signal is presented in cyan. Images are acquired using epifluorescence microscopy. Representative sagittal images of left hemispheres are displayed (N=3 mice per condition). Section thickness=30 μm, scale bars=2.5 mm. Histological examination of distribution in spinal cord either 2 or 48 hours after ICV injection of unconjugated siRNA, Chol-siRNA, or L2-siRNA (7.5-10 nmol dose) (right side panels). The 2-hour time point shows representative lumbar regions while the 48-hour time point shows representative cervical regions (N=3 mice per condition). Section thickness=30 μm, scale bars=500 μm.

FIG. 2E: Plot profile of Cy5 signal emanating from ventral subarachnoid CSF 2 hours after injection. Yellow arrow in panel D (labeled as “E”) represents the analyzed region. Data are presented as mean of N=3 biological replicates.

FIG. 2F: Representative plot profile of Cy5 signal across deeper brain structures 48 hours after injection (from midbrain to interpeduncular cistern). Arrow in panel D (labeled as “F”) represents the analyzed region. Arrows point to L2-siRNA peaks reflecting perivascular transport far from brain boundaries.

FIG. 2G: Representative confocal image showing perivascular localization of L2-siRNA 48 hours after injection. Perivascular space is defined as the region between Glut1+ endothelium and AQP4+ glia limitans (stained with fluorescently conjugated antibodies). Scale bar=20 μm (left) or 10 μm (right). The inset is acquired at the indicated area but at a slightly different z-plane using confocal microscopy.

FIG. 2H: Representative traces of Cy5 fluorescence within the perivascular space (PVS) shown for a representative vessel from each condition, with boundaries demarcated by peak Glut1 and Aqp4 intensities.

FIG. 2I: Quantification of perivascular Cy5 signal. Each dot represents the average of 5 non-capillary vessels from a single mouse. N=3 biological replicates per condition and data presented as mean±SD. Statistical significance was calculated using a one-way ANOVA with Bonferroni's correction (** p<0.01, ns—not significant). AU=arbitrary units, AUC=area under curve.

FIG. 3A: Experimental approach for evaluating gene silencing. Mice receive a bilateral ICV injection of 15 nmol L2-siRNA^{NTC}, siRNA^{Htt}, or L2-siRNA^{Htt} and brain tissue is harvested after 1, 3, or 5 months. The brain is sliced into 1 mm sagittal sections, which are biopsy punched (2 mm) to extract different regions for analysis. A representative brain section is shown with biopsy punches extracted. RT-qPCR samples are acquired from the first 1 mm medial slice of the left hemisphere, Western blot samples are acquired from the second medial slice of the left hemisphere, and PNA samples are acquired from the first medial slice of the right hemisphere. Str, striatum; Hip, hippocampus; Cor, cortex; Bstem, brainstem; Cere, cerebellum.

FIG. 3B-FIG. 3D: Htt mRNA levels in parenchymal regions as measured by RT-qPCR. Each region and time point is normalized to its respective L2-siRNA^{NTC} control. For 1 (FIG. 3B) and 3 months (FIG. 3C), significance was calculated for each region and timepoint as a one-way ANOVA compared to L2-siRNA^{NTC} with Bonferroni's correction for multiple comparisons (N=5-6 mice). For 5 months (FIG. 3D), unpaired two-tailed t-tests were performed for each region (N=7-8 mice).

FIG. 3E-FIG. 3G: Htt protein levels in parenchymal regions as measured by Western blot. Each region and time point is normalized to its respective L2-siRNA^{NTC} control. For 1 (FIG. 3E) and 3 months (FIG. 3F), significance was calculated for each region and timepoint as a one-way ANOVA compared to L2-siRNA^{NTC} with Bonferroni's correction for multiple comparisons (N=5-6 mice). For 5 months (FIG. 3G), unpaired two-tailed t-test was performed for each region (N=7-8 mice).

FIG. 3H: Absolute amount of antisense strand (ng) per milligram of brain tissue as measured by the PNA assay for L2-siRNA^{NTC}. N=4-6 biological replicates per region per time point (M=month). For all panels, data are represented as mean±SD and each point represents an individual biological replicate (i.e. a single mouse brain). In all panels, statistical significance has the same labels (* p<0.05, ** p<0.01, *** p<0.001, **** p<0.0001, ns—not significant).

FIG. 4A: Htt expression levels in spinal cord regions as measured by RT-qPCR. Mice receive a bilateral ICV injection of 15 nmol L2-siRNA^{NTC}, siRNA^{Htt}, or L2-siRNA^{Htt} and spinal cords collected after 1, 3, or 5 months. N=4-6 spinal cords analyzed at 1 and 3 months, and N=7-8 spinal cord analyzed at 5 months. For 1 and 3 months, significance was calculated for each region and timepoint as a one-way ANOVA compared to the L2-siRNA^{NTC} control with Bonferroni's correction for multiple comparisons. For 5 months, an unpaired two-tailed t-test was performed for each region.

FIG. 4B: Htt expression levels in spinal cord regions as measured by Western blot. N=4-6 spinal cords analyzed at 1 and 3 months, and N=7-8 spinal cord analyzed at 5 months. For 1 and 3 months, significance was calculated for each region and timepoint as a one-way ANOVA compared to the L2-siRNA^{NTC} control with Bonferroni's correction for multiple comparisons. For 5 months, unpaired two-tailed t-test was performed for each region.

FIG. 4C: Absolute amount of antisense strand (ng) per milligram of spinal cord tissue measured by the PNA assay at 1, 3, and 5 months (M). N=3-6 biological replicates per region per time point. Values below the limit of detection were plotted as 0.

FIG. 4D: Relationship between L2-siRNA delivery and gene/protein knockdown. For every graph, each point represents an individual biological replicate (i.e. a single mouse spinal cord) and data are presented as mean±SD. In all panels, statistical significance has the same labels (* p<0.05, ** p<0.01, *** p<0.001, **** p<0.0001, ns—not significant).

FIG. 5A: Experimental design for assessment of cell-specific gene silencing. Mice were administered 15 nmol of L2-siRNA^{Ppib} or L2-siRNA^{NTC} and after 1 month, brains were dissociated into single cells and bead-sorted into Cd11b^{pos} and Cd11b^{neg} populations, which were further separated for scRNA-seq or RT-qPCR. For all experiments in this figure, N=3 individual mice (biological replicates) for both L2-siRNA^{Ppib} or L2-siRNA^{NTC}.

FIG. 5B: Ppib expression in Cd11b^{pos} and Cd11b^{neg} populations measured by RT-qPCR. Statistical significance was calculated using an unpaired two-tailed t-test (** p<0.01, *** p<0.001).

FIG. 5C: Comparison of Ppib expression between bulk RT-qPCR (x-axis) and scRNA-seq averaged across all identified cells (y-axis). Line with a slope of 1 represents equivalent expression between readouts.

FIG. 5D: Uniform Manifold Approximation and Projection (UMAP) dimension reduction plot visualizes clusters of cell populations. Here, it is shown that all Cd11b^{pos} populations were identified, where each cell is a dot and each population is presented in a different color. These clusters show a clear grouping of different cell types.

FIG. 5E: Knockdown in CD11b^{pos} cells. Statistical significance was calculated using an unpaired two-tailed t-test (** p<0.01).

FIG. 5F: UMAP dimension reduction plot showing clustering and annotation of Cd11b^{neg} populations. Unless indicated with a line, all text labels lie above their respective cluster. Each cell cluster is presented in a different color to highlight their unique identity.

FIG. 5G: Average Ppib expression across different Cd11b^{neg} cell types, normalized to L2-siRNA^{NTC}. Statistical significance was calculated using an unpaired two-tailed t-test (* p<0.05, ** p<0.01, *** p<0.001, **** p<0.0001, ns—not significant). Data are presented as mean±SD in every panel.

FIG. 6A: Depiction of fibroblast anatomical locations in the leptomeninges.

FIG. 6B: UMAP dimension reduction plot showing sub-clustering of fibroblast populations. Each cluster is presented in a different color to emphasize the unique gene expression profile (i.e., each cell population groups together).

FIG. 6C: scRNA-seq assessment of Ppib knockdown in fibroblasts of the brain borders at a 1-month time point. Knockdown is normalized to L2-siRNA^{NTC}. N=3 individual mice (biological replicates) for both L2-siRNA^{Ppib} and L2-siRNA^{NTC}. Statistical significance was calculated using an unpaired two-tailed t-test (* p<0.05, ** p<0.01, *** p<0.001).

FIG. 6D: Light sheet microscopy is used to generate a 3D image demonstrating extensive perivascular delivery of L2-siRNA (magenta) around lectin-stained vessels (green) in the cortex (48 hours, 10 nmol dose). Representative images displayed are consistent across replicates.

FIG. 6E: Representative confocal images show delivery of Cy5-tagged L2-siRNA to Lyve1⁺ border-associated macrophages (BAM) shown in cyan. (I) Delivery along penetrating vessel, scale bar=20 μm. (II, III) Subcellular localization in perivascular Lyve1+ BAMs, scale bar=10 μm. (IV) Delivery to ventral meningeal Lyve1+ BAMs, scale bar=50 μm.

FIG. 6F: Representative confocal images of Cy5-tagged L2-siRNA delivery to MHCII+ BAMs 48 hours after injection. Scale bar=100 μm (top left), 50 μm (top right), 25 μm (bottom).

FIG. 6G: scRNA-seq assessment of Ppib knockdown in MHCII⁺ macrophages at a 1-month time point. Knockdown is normalized to L2-siRNA^{NTC}. N=3 individual mice (biological replicates) for both L2-siRNA^{Ppib} and L2-siRNA^{NTC}. Statistical significance was calculated using an unpaired two-tailed t-test (*** p<0.001). Data are presented as mean±SD for all graphs.

FIG. 6H: Representative confocal image of Cy5-tagged L2-siRNA delivery to cells of the 4th ventricle choroid plexus (ChP) 48 hours after injection (2 nmol in 10 μl ICV). An antibody against Aqp1 is used to mark the apical side of ChP epithelial cells, while a CD31 antibody stained the inner vasculature. Scale bar=100 μm (top) and 50 μm (bottom).

FIG. 6I: scRNA-seq assessment of Ppib knockdown in ChP epithelial cells at a 1-month time point. Knockdown is normalized to L2-siRNA^{NTC}. N=3 individual mice (biological replicates) for both L2-siRNA^{Ppib} and L2-siRNA^{NTC}. Statistical significance was calculated using an unpaired two-tailed t-test (**** p<0.0001).

FIG. 7A: Mice administered 15 nmol of compounds (or 0.9% saline) and stained with a GFAP antibody (shown in green) 1-month after ICV injection to visualize reactive astrogliosis (as indicated by elevated GFAP signal). Representative images of the hippocampal dentate gyrus are shown. Section thickness=8 μm, scale bar=100 μm.

FIG. 7B: Quantification of GFAP mean fluorescence intensity (MFI) normalized to vehicle across different brain regions. Data are presented as mean±SD, with N=4-5 mice per condition. One-way ANOVA with Bonferroni's correction was performed for each region.

FIG. 7C: GFAP levels measured by Western blot, normalized to GADPH housekeeping gene, and then normalized to vehicle control. Raw Western blot displayed for GFAP and GAPDH. N=5 mice statistically analyzed with a one-way ANOVA and Bonferroni's correction.

FIG. 7D: Mice were administered 15 nmol of compounds (or 0.9% saline) and stained with an antibody for Iba1 1-month after ICV injection. The abundance of Iba1 reflects microglia proliferation, a hallmark of neuroinflammation. Representative images of the hippocampal dentate gyrus are shown. Section thickness=8 μm, scale bar=100 μm.

FIG. 7E: Quantification of Iba1 protein levels normalized to vehicle. N=4-6 mice per condition. One-way ANOVA with Bonferroni's correction was performed for each region compared to vehicle.

FIG. 7F: Levels of inflammatory cytokines assessed two weeks after ICV injection of vehicle (0.9% saline) or L2-siRNA^{Htt} (5 nmol or 15 nmol). N=4 mice per condition. One-way ANOVA with Bonferroni's correction was performed for each region and cytokine (ns—not significant). All data are presented as mean±SD.

FIG. 8A: Cell uptake was evaluated by flow cytometry in N2a cells after a 2-hour incubation with unconjugated siRNA, Chol-siRNA, or L2-siRNA (60 nM). Mean fluorescence intensity (MFI) is reported from three independent experiments, where the bars represent mean±SD. One-way ANOVA without assuming equal SD was performed with Dunnett's T3 correction for multiple comparisons (*p<0.05).

FIG. 8B: Carrier-free Htt knockdown in N2a cells assessed by RT-qPCR after a 48-hour incubation with siRNA^{Htt}, Chol-siRNA^{Htt}, or L2-siRNA^{Htt} normalized to L2-siRNA^{NTC} (represented by dotted line at Y=1.0). Data reported from two independent experiments, each normalized to L2-siRNA^{NTC}, with bars representing mean±SD. One-way ANOVA compared to L2-siRNA^{NTC} with Bonferroni's multiple comparison correction (ns—not significant, ** p<0.01).

FIG. 8C: Albumin-binding properties of siRNA conjugates in human CSF. Cy5-labeled siRNA, Chol-siRNA, or L2-siRNA (1 μM) mixed with 300 μl of human CSF was analyzed using fast protein liquid chromatography (FPLC). Only L2-siRNA elutes in known albumin-containing fractions. Same N=1 CSF sample used for each condition.

FIG. 8D: L2-siRNA associates with albumin in CSF from patients with Alzheimer's Disease (AD) as well as those without neurodegeneration (N=3 control and AD samples). Error bars represent SD.

FIG. 9A-FIG. 9B: Accumulation in peripheral organs assessed by In Vivo Imaging System (IVIS) 2 hours (FIG. 9A) and 48 hours (FIG. 9B) after ICV injection of Cy5-labeled siRNA or L2-siRNA (7.5-10 nmol). Average radiant efficiency of Cy5 fluorescence is reported. N=3-4 mice.

FIG. 9C: L2-siRNA^{NTC} liver delivery 1, 3 and 5 months (M) after ICV injection (15 nmol) measured with the PNA assay. Values below the limit of detection were plotted as 0. N=4-6 mice.

FIG. 9D: Htt mRNA expression levels in the liver as measured by RT-qPCR 3 months after ICV injection (15 nmol). Each point represents an individual mouse (N=3-6). Significance was calculated as a one-way ANOVA compared to L2-siRNA^{NTC} with Bonferroni's correction for multiple comparisons. Data presented as mean±SD in every graph (ns—not significant).

FIG. 10A: Comparing siRNA delivery between L2-siRNA^{NTC} and L2-siRNA^{Htt} 3 months after ICV injection (15 nmol). Units are nanograms (ng) of anti-sense strand per milligram (mg) of tissue. Mean±SD from N=5-6 biological replicates, two-tailed unpaired t-tests for each region (ns—not significant, *p<0.05).

FIG. 10B: Representative HPLC trace showing elution of anti-sense: PNA complex overlayed with ion-exchange gradient (dashed black line). Trace shown from a cortex sample of L2-siRNA^{NTC}.

FIG. 11: Mice were administered 15 nmol of ASO^{Htt} or L2-siRNA^{Htt} ICV and knockdown was normalized to a vehicle (0.9% NaCl) control. The ASO^{Htt} sequence was designed and ordered from IDT. Htt levels were determined by RT-qPCR one month after injection. Since the ASO^{Htt} and L2-siRNA^{Htt} knockdown studies were performed on different days, each sample is normalized to their respective cohort of vehicle controls, and only the vehicle from the ASO^{Htt} study is plotted here. Each point represents a mouse (N=6) and bars represent mean±SD. One-way ANOVA was performed for each region comparing ASO^{Htt} to vehicle, L2-siRNA^{Htt} to ASO^{Htt} (Bonferroni's correction for multiple comparison).

FIG. 12A-FIG. 12G: Mice were injected ICV with a vehicle (0.9% NaCl) or L2-SiRNA^{Htt} (5 nmol or 15 nmol), and blood serum was isolated after two weeks. Six standard markers were measured in this panel: alanine aminotransferase (ALT), aspartate aminotransferase (AST), blood urea nitrogen (BUN), creatinine, amylase, and total bilirubin. Data are presented as mean±SD. Statistics are computed as a one-way ANOVA with Bonferroni's correction compared to vehicle (ns—not significant, *p<0.05). Standard chemistry reference range is represented by dotted lines.

FIG. 13A: Experimental design to assess endothelial uptake of L2-siRNA. In brief, mice are injected with 20 mg/kg of Cy5 labeled L2-siRNA or unconjugated siRNA and after 48 hours, the brain is apportioned for flow cytometry and histology.

FIG. 13B: Cell specific antibodies are used to identify CNS cells types, and Cy5 uptake is quantified as mean fluorescence intensity or gated for percent positive based on fluorescent-minus-one controls (i.e., brain cells from an uninjected mouse). Statistics computed as multiple unpaired two-tailed t-tests (Holm-Sidak), and bar represent mean±SD. (* p<0.05, ** p<0.01, *** p<0.001, **** p<0.0001).

FIG. 13C: Epifluorescence microscopy is used to visualize L2-siRNA delivery throughout the mouse brain, revealing widespread accumulation in structures resembling blood vessels. Delivery to the brainstem (bottom left, scale bar=500 μm) and hippocampus (bottom right, scale bar=200 μm) are highlighted. Section thickness=20 μm, scale bar=2 mm for hemisphere image.

FIG. 13D: To discern L2-siRNA localization, histological samples are stained for endothelial cells (CD31+ inner portion of vessel) and laminin (reflects outer border of the neurovascular unit).

FIG. 14A: 48 hours after a 20 mg/kg I.V. injection of Cy5 labeled siRNA and L2-siRNA, the 4th ventricle, lateral ventricle, and 3^{rd} ventricle choroid plexus were extracted, embedded, and sectioned. Representative epifluorescence images of delivery to choroid plexus.

FIG. 14B: Quantification of Cy5 mean fluorescence intensity (MFI) in each choroid plexus shows equivalent delivery to each choroid plexus, with far greater accumulation achieved for L2-siRNA compared to unconjugated siRNA. Statistics represent unpaired two-tailed t-tests, mean±SD. (** p<0.01, *** p<0.001, **** p<0.0001).

FIG. 14C: The 4^{th} ventricle ChP is whole mounted (confocal z-stack projection, scale bar=2 mm) and labeled with aquaporin 1 (AQP1) staining the apical side of the epithelium and CD31 staining the vasculature. This image highlights the broad distribution of L2-siRNA throughout the choroid plexus.

FIG. 14D: Confocal microscopy used to show L2-siRNA localization in the stromal space and in AQP1+ epithelial cells.

FIG. 14E: Punctate pattern observed in ChP vessels highlights delivery of L2-siRNA to the vasculature.

FIG. 14F: Bright L2-siRNA signal colocalizes with Iba1+ macrophages, as visualized by confocal microscopy.

FIG. 15A: Experimental design to assess Ppib silencing in brain endothelial cells and the choroid plexuses.

FIG. 15B: Time course of mRNA knockdown in MACS sorted endothelial cells as assessed by RT-qPCR. Results are normalized to non-targeting control (NTC) L2-siRNA^{NTC} at each time point examined.

FIG. 15C: Time course of knockdown in non-endothelial CNS cells (CD31^{neg}).

FIG. 15D: Gene silencing in the choroid plexuses (ChPs) of the two lateral ventricles combined.

FIG. 15E: Gene silencing in the ChP of the fourth ventricle. For all panels, N=5 biological replicates (i.e. individual mice) for each timepoint. Unpaired two-tailed t-tests for each timepoint normalized to L2-siRNA^{NTC}. NTC=non-targeting control, mean±SD. (* p<0.05, ** p<0.01, *** p<0.001, **** p<0.0001, ns—not significant).

FIG. 16A: Carrier-free gene silencing for sequence 1152 assessed in RAW 264.7 macrophages after 48 hours. Data shown is a representative experiment containing N=3 technical replicates (i.e. treated wells), and normalized to a non-targeting L2-siRNA control.

FIG. 16B: Carrier-free gene silencing for sequence 2288 assessed in RAW 264.7 macrophages after 48 hours. Data shown is a representative experiment containing N=3 technical replicates (i.e. treated wells), and normalized to a non-targeting L2-siRNA control.

FIG. 16C: Experimental design to assess CD33 silencing. Mice are injected with 15 nmol of siRNA and the brain is harvested after one month, where some tissue is collected via biopsy punch to examine regional knockdown and the remaining tissue is used to isolate microglia (CD11b^{pos}).

FIG. 16D: Both CD33-targeting constructs exhibit robust gene silencing in CD11b isolated cells, as measured by RT-qPCR.

FIG. 16E: The spinal cord was isolated and segmented for mRNA analysis.

FIG. 16F: Gene silencing in five regions of the parenchyma. All statistical tests in D-F are one-way ANOVA with Bonferroni's correction. N=6 mice, data presented as mean±SD. (* p<0.05, ** p<0.01, *** p<0.001, **** p<0.0001, ns—not significant).

FIG. 17A: Identifying candidate zipper siRNA sequences in Bend.3 cells by RT-qPCR. Sequences are named by the mRNA target position. Representative reverse transfection shown where each data point represents a technical replication (i.e., a well).

FIG. 17B: Four lead candidates were selected from the lipofectamine screen and were synthesized as L2-siRNA. These constructs were screened carrier-free at two doses in Bend.3 brain endothelial cells.

FIG. 17C: Sequence “573” was chosen and evaluated in vivo for gene silencing. The interleukin 1 receptor, type I (IL-1R1) targeting L2-siRNA conjugate (L2-siRNA^{IL-1R1}) was injected 20 mg/kg on day 1 and 4 and compared to a non-targeting control (L2-siRNA^{NTC}). At day 14, mice were injected ICV with IL1β or saline.

FIG. 17D: IL-1R1 gene levels are measured in CD31 MACS sorted cells. N=5 mice. Unpaired two-tailed t-tests (Holm-Sidak correction).

### DETAILED DESCRIPTION

Neurodegenerative disease is one of the greatest threats to independence and quality of life in the elderly. Yet, despite considerable advances in understanding disease pathophysiology and the identification of novel therapeutic targets, most age-associated neurodegenerative diseases remain incurable. This incongruency may be attributed, in part, to the challenge of modulating expression of disease-driving proteins, many of which are considered undruggable by traditional technologies such as small molecule or antibody therapies. This is either due to their structure (e.g., necessity of binding pockets for small molecules, or surface expression for antibody engagement) or their location within the central nervous system (e.g., requires crossing the blood-brain-barrier (BBB)). These shortcomings highlight the unmet need for strategies that achieve therapeutic dosing of biological drugs in the brain without negative side effects.

Gene therapies, in particular short interfering RNA (siRNAs), are an emerging class of therapeutics that have generated great interest in the clinic, owing to their ability to inhibit nearly any target. Despite the recent FDA approval of four new siRNA drugs (all targeting hepatocytes), extrahepatic targets remain underdeveloped, as there are no siRNA therapies currently approved for CNS disorders. The central challenge is one of drug delivery-first into the brain parenchyma and then into specific cells. Unfortunately, at present, systemic administration of siRNA-based therapeutics do not reach the CNS in meaningful concentrations due to the blood-brain barrier (BBB). To circumvent the BBB, drugs can be injected into the CSF, leading to distribution in brain regions connected to bulk CSF circulation. This is the most common delivery route for oligonucleotide drugs, yet brain-wide distribution remains subpar because CSF clearance is rapid (˜5-6 hour turnover in humans) and siRNA does not effectively enter cells owing to its negatively charged backbone. Thus, to advance clinically relevant lipid-siRNA biotechnology, a balance must be achieved between transport through CSF compartments (including perivascular spaces), dispersion through parenchymal tissue, and sufficient cell uptake, all without overt toxicity.

Disclosed herein is the use of a lipid-siRNA conjugate that can overcome the aforementioned shortcomings by being able to potentiate widespread and long-term gene silencing in the CNS. Specifically, it has been found that the disclosed methods can achieve high transfection of cells in the brain; uptake in specific locations of the CNS, such as the choroid plexus and perivascular spaces; and long-term silencing 30 days post-administration and beyond.

### 1. Definitions

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed technology. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are contemplated, and for the range 1.5-2, the numbers 1.5, 1.6, 1.7, 1.8, 1.9, and 2 are contemplated.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “antisense strand” refers to a strand of an siRNA duplex that contains some degree of complementarity to an oligonucleotide and contains complementarity to the sense strand of the siRNA duplex.

The term “attached” refers to two moieties being attached through a bond where there can be intervening moieties or molecules in between. For example, the branching molecule can be attached to the siRNA by having an intervening moiety, such as a second linker, between the siRNA and the linker. Attached can also include “directly attached,” which refers to two moieties being attached through a bond with no other intervening moieties or molecules.

The terms “effective amount” or “therapeutically effective amount” refer to an amount sufficient to effect beneficial or desirable biological and/or clinical results to treat a disease or one or more of its symptoms and/or to prevent or reduce the risk of the occurrence or reoccurrence of the disease or disorder or symptom(s) thereof. In reference to central nervous system diseases an effective or therapeutically effective amount can include an amount sufficient to, among other things, inhibit the expression of a protein associated with the central nervous system disease. Inhibiting the expression of a protein associated with the central nervous system disease may decrease the underlying pathology associated with the central nervous system disease.

The term “complementary” refers to the relationship between nucleotides exhibiting Watson-Crick base pairing, or to oligonucleotides that hybridize via Watson-Crick base pairing to form a double-stranded nucleic acid. The term “complementarity” refers to the state of an oligonucleotide (e.g., a sense strand or an antisense strand) that is partially or completely complementary to another oligonucleotide. Oligonucleotides described herein as having complementarity to a target oligonucleotide may be ˜ 100%, >99%, >95%, >90%, >85%, >80%, >75%, >70%, >65%, >60%, >55% or >50% complementary to the target oligonucleotide. Complementary and complementarity can also be used to describe specifically hybridizing to a target oligonucleotide by a siRNA.

The term “oligonucleotide” refers to a polymer of nucleotides. The terms “polynucleotide,” “nucleic acid,” and “oligonucleotide”, may be used interchangeably herein. Typically, a polynucleotide comprises at least three nucleotides. Oligonucleotides can be single stranded or double stranded. Example oligonucleotides include, but are not limited to, DNA and RNA, such as mRNA, RNAi, siRNA, and shRNA.

The term “sense strand” refers to a strand of an siRNA duplex that contains complementarity to an antisense strand of the siRNA duplex.

The term “siRNA” refers to small interfering RNAs that induce the RNA interference (RNAi) pathway. siRNA molecules can vary in length (e.g., 10-40 base pairs) and contain varying degrees of complementarity to their target oligonucleotide. The term “siRNA” can include duplexes of two separate strands, as well as single strands that can form hairpin structures comprising a duplex region.

The terms “treatment” or “treating” refer to the medical management of a subject with the intent to heal, cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### 2. Methods

Disclosed herein are methods of treating a central nervous system (CNS) disease in a subject. The method can include administering to the subject an effective amount of a conjugate. The conjugate can be administered optionally with a pharmaceutically acceptable excipient. The conjugate can include a siRNA capable of inhibiting expression of a protein associated with the CNS disease; a lipophilic ligand capable of binding albumin; and a linker attaching the siRNA to the lipophilic ligand, the linker including a branching molecule attached to the siRNA, and a hydrophilic spacer attaching the branching molecule to the lipophilic ligand.

A number of CNS diseases may be treated by the disclosed methods. For example, the CNS disease can be Alzheimer's disease, Huntington's disease, a tauopathy, frontal temporal dementia, hydrocephalus, stroke, a brain tumor, or Amyotrophic Lateral Sclerosis. In some embodiments, the CNS disease is Alzheimer's disease, Huntington's disease, or Amyotrophic Lateral Sclerosis. In some embodiments, the CNS disease is Alzheimer's disease or Huntington's disease. In some embodiments, the CNS disease is Huntington's disease.

The subject of the disclosed methods is generally not limited and can be any animal that is in need of a treatment for a CNS disease. Subject can mean a mammal that wants or is in need of the herein described conjugates or methods. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant. The subject may be male or female. In some embodiments, the subject is human.

The methods can provide advantageous benefits to the subject receiving the treatment. For example, the method can decrease the underlying pathology associated with the CNS disease in the subject. Underlying pathology as it relates to the CNS disease can include causes, developments, and/or structural/functional changes associated with the CNS disease and its progression. The underlying pathology for individual CNS diseases can differ, and it is within the skill of the artisan to recognize different underlying pathologies for different diseases. For example, Huntington's disease (HD) is a promising candidate for gene inhibition therapies because it is a monogenic disorder caused by polyglutamine expansions solely in the huntingtin (Htt) protein. There is a considerable push to use precise molecular tools, such as an siRNA, to treat Huntington's disease by silencing expression of this pathological protein. Similarly, advances in the understanding of underlying Alzheimer's Disease mechanisms has led to the identification of new therapeutic targets. Some of these targets, (such as CD33, beta-secretase, and amyloid precursor protein) seek to mitigate amyloid plaque build-up, while others are aimed at reducing tau pathology (i.e., MAPT inhibition).

In some embodiments, the method decreases the underlying pathology associated with the CNS disease for at least 60 days post-administration, at least 40 days post-administration, at least 30 days post-administration, at least 25 days post-administration, at least 20 days post-administration, at least 15 days post-administration, or at least 10 days post-administration. In some embodiments, the method decreases the underlying pathology associated with the inflammatory disease for greater than 5 days post-administration, greater than 10 days post-administration, greater than 15 days post-administration, greater than 20 days post-administration, greater than 25 days post-administration, or greater than 30 days post-administration.

The methods can provide widespread gene silencing in the CNS of the subject. For example, the method can inhibit the expression of the protein associated with the CNS disease in the subject's striatum, hippocampus, cortex, cerebellum, spinal cord, brain parenchyma, brain vasculature, choroid plexus, brainstem, or a combination thereof. In some embodiments, the method inhibits the expression of the protein associated with the CNS disease in the subject's striatum, hippocampus, cortex, cerebellum, spinal cord, brain parenchyma, brain stem, or a combination thereof. In some embodiments, the method inhibits the expression of the protein associated with the CNS disease in the subject's brain vasculature, choroid plexus, or both.

The method can also provide long-lasting gene silencing in the CNS of the subject. For example, the method can inhibit the expression of the protein associated with the CNS disease for at least 1 month post-administration, at least 2 months post-administration, at least 3 months post-administration, at least 4 months post-administration, at least 5 months post-administration, or at least 6 months post-administration. In some embodiments, the method inhibits the expression of the protein associated with the CNS disease for about 1 month to about 6 months, such as about 1 month to about 5 months, about 2 months to about 5 months, or about 3 months to about 5 months. In some embodiments, the aforementioned long-lasting gene silencing is done with a single administration of the conjugate.

Numerous techniques known within the art can be used to assess the associated benefits of the disclosed methods, such as gene or protein expression (e.g., RT-qPCR, Western blot, etc.) of target molecules and histology of a site associated with the CNS disease. Further discussion of assessing the disclosed methods can be found herein, e.g., in the Examples.

In another aspect, disclosed are methods of delivering a therapeutic to a CNS of a subject (e.g., in need thereof). The method can include administering the conjugate as disclosed herein to the subject intravenously or intracerebroventricularly. The method can include the conjugate localizing to the subject's CNS. For example, the conjugate can localize to the subject's striatum, hippocampus, cortex, cerebellum, spinal cord, brain parenchyma, brain vasculature, choroid plexus, brain stem, or a combination thereof. It has been found that the administration route can affect the localization of the conjugate. For example, it has been found that systemic intravenous administration can allow localization of the conjugate to the choroid plexus or brain vasculature, while local intracerebroventricular can allow localization to the perivascular regions of the brain. In some embodiments, the conjugate is administered intravenously and localizes to the subject's brain vasculature, choroid plexus, or both. In some embodiments, the conjugate is administered intracerebroventricularly and localizes to the subject's perivascular region of the brain.

### A. Conjugates

The conjugate can provide advantageous benefits for the disclosed methods. For example, the conjugate, following administration, can localize to regions of the CNS associated with the CNS disease. This can allow for a more targeted, long-lasting treatment of the CNS disease, while decreasing potential side-effects within the subject.

The conjugate includes a siRNA, a lipophilic ligand capable of binding albumin, and a linker attaching the siRNA and the lipophilic ligand. The linker includes a branching molecule attached to the siRNA and a hydrophilic spacer attaching the branching molecule to the lipophilic ligand.

The siRNA, the linker, and the lipophilic ligand can be attached to each other through various types of linkages/bonds. For example, the siRNA, the linker, and/or the lipophilic ligand can be attached through phosphorothioate bonds, phosphodiester bonds, a cleavable linker (e.g., deoxythymidine (dT), pH-cleavable bond such as ketal), or a combination thereof. In some embodiments, the siRNA, the linker, and/or the lipophilic ligand are attached through phosphorothioate bonds. In some embodiments, the conjugate includes about 20% to about 60% phosphorothioate linkages based on the total amount of phosphate-based linkages in the conjugate, such as about 20% to about 55% phosphorothioate linkages, about 25% to about 50% phosphorothioate linkages, about 35% to about 45% phosphorothioate linkages, or about 40% to about 45% phosphorothioate linkages-based on the total amount of phosphate-based linkages in the conjugate. The combination of phosphodiester linkages and phosphorothioate linkages can be referred to as the total amount of phosphate-based linkages, and is not inclusive of potential phosphorylation of sequences, e.g., to avoid deactivation of phosphatases.

The conjugate's arrangement and composition can provide advantageous benefits, such as being able to bind albumin while also minimizing its propensity to self-assemble into micelles. For example, the conjugate can have a binding affinity (K_{d}) to albumin of less than 1 μM, less than 500 nM, less than 250 nM, less than 100 nM, less than 80 nM, less than 60 nM, less than 50 nM, less than 45 nM, less than 40 nM, or less than 35 nM. In some embodiments, the conjugate has a K_{d} to albumin of greater than 0.1 nM, greater than 0.2 nM, greater than 0.4 nM, greater than 0.5 nM, greater than 0.6 nM, greater than 0.7 nM, greater than 0.8 nM, greater than 0.9 nM, greater than 1 nM, or greater than 5 nM. In some embodiments, the conjugate has a K_{d} to albumin of about 0.1 nM to about 1 μM, such as about 0.5 nM to about 500 nM, about 0.8 nM to about 100 nM, about 0.5 nM to about 100 nM, about 1 nM to about 50 nM, or about 5 nM to about 35 nM. The conjugate can reversibly bind albumin. In some embodiments, the conjugate does not covalently bind to albumin.

In addition, the conjugate can have a critical micelle concentration of greater than 1850 nM, greater than 1900 nM, greater than 1950 nM, greater than 2000 nM, greater than 2100 nM, greater than 2200 nM, greater than 2300 nM, greater than 2400 nM, greater than 2500 nM, greater than 3000 nM, or greater than 3500 nM. In some embodiments, the conjugate has a critical micelle concentration of less than 4500 nM, less than 4000 nM, less than 3500 nM, less than 3000 nM, less than 2500 nM, less than 2400 nM, less than 2300 nM, less than 2200 nM, less than 2100 nM, less than 2000 nM, or less than 1950 nM. In some embodiments, the conjugate has a critical micelle concentration of about 1850 nM to about 4000 nM, such as about 1900 nM to about 3500 nM, about 2000 nM to about 3500 nM, about 1850 nM to about 3500 nM, or about 2500 nM to about 3500 nM.

As discussed elsewhere, the conjugate can bind albumin through the lipophilic ligand. In some embodiments, the binding of the conjugate to the serum protein albumin enhances the pharmacokinetic properties of the siRNA as compared to an unmodified siRNA and/or existing nanocarrier including the siRNA. In some embodiments, enhancing the pharmacokinetic properties includes increasing the circulation half-life and/or bioavailability of the siRNA, as compared to an unmodified siRNA and/or existing nanocarrier including the siRNA. Additionally, enhancing the pharmacokinetic properties may including improving localization to specific tissues, increasing the quantity of cellular accumulation, increasing the homogeneity of cellular accumulation, increasing resistance to nucleases, and/or permitting increased dosing amount with decreased toxicity as compared to an unmodified siRNA and/or existing nanocarrier including the siRNA.

i. SiRNA

As mentioned above, the conjugate includes a siRNA. The siRNA can instill a therapeutic and/or beneficial property to the conjugate. The siRNA can be single stranded or double stranded. An example of a single stranded siRNA includes, but is not limited to, single stranded antisense RNA.

The benefits realized from the end modification of the disclosed conjugate (e.g., attaching the lipophilic ligand to the siRNA through the linker) can be used with any desirable siRNA useful for treatment of a CNS disease. In other words, the siRNA of the conjugate is sequence agnostic. This modification can be added to either single or double stranded siRNA that contain either natural or modified nucleotide bases. The single or double stranded nucleotides can also be of variable length, such as 10 to 40 bases in length.

For an example process of selecting an siRNA, the sequence can be first determined using publicly available prediction algorithms. These algorithms can generate many candidate sequences for targeting any given gene. These potential sequences are first screened for on-target gene silencing potency in vitro. After identification of one or more potent sequences, L2 chemical modifications can be added to the sequence, and it can be rescreened for in vitro gene silencing activity prior to screening for albumin binding affinity and pharmacokinetic/pharmacodynamic behaviors in vivo. The disclosed albumin binding end chemistry may be successfully integrated with multiple sequences targeting any single gene and may also be adapted for delivery of sequences against theoretically any gene of interest.

The siRNA can be capable of specifically hybridizing to an oligonucleotide encoding a protein associated with a signaling pathway involved with the pathology of the CNS disease. In some embodiments, the siRNA is capable of specifically hybridizing to an oligonucleotide encoding an amyloid protein, a tau protein, or an oncogene. In some embodiments, the siRNA is capable of specifically hybridizing to an oligonucleotide encoding huntingtin, CD33, ApoE, MAPT, PDK1, C1QA, VCAM1, TREM2, SPP1, C3, SOD1, SERPINA3, IL-1R1, PPIB, or Htt. In some embodiments, the siRNA is capable of specifically hybridizing to an oligonucleotide encoding huntingtin, CD33, ApoE, MAPT, PDK1, C1QA, VCAM1, TREM2, SPP1, C3, SOD1, SERPINA3, or IL-1R1. In some embodiments, the siRNA is capable of specifically hybridizing to an oligonucleotide encoding huntingtin, CD33, ApoE, MAPT, PDK1, C1QA, VCAM1, TREM2, SPP1, or C3. In some embodiments, the siRNA is capable of specifically hybridizing to an oligonucleotide encoding huntingtin, CD33, or IL-1R1.

The siRNA can include a plurality of stabilizing modifications. Examples of stabilizing modifications include, but are not limited to, phosphorothioate linkages, 2′F modification, 2′OMe modification, and combinations of 2′F and 2′OMe modifications (e.g., zipper pattern). In addition, the siRNA can include both phosphodiester linkages and phosphorothioate linkages. In some embodiments, the siRNA includes a plurality of phosphorothioate linkages. In some embodiments, the siRNA includes phosphorothioate linkages at its terminal end(s). In some embodiments, the siRNA includes about 1% to about 30% phosphorothioate linkages based on a total amount of phosphate-based linkages in the siRNA, such as about 10% to about 25% phosphorothioate linkages or about 15% to about 24% phosphorothioate linkages-based on a total amount of phosphate-based linkages in the siRNA. In some embodiments, the siRNA includes about 21% phosphorothioate linkages based on a total amount of phosphate-based linkages in the siRNA.

The siRNA can have a varying number of nucleotides. For example, the siRNA can be about 15 to about 40 nucleotides in length, such as about 16 to about 38 nucleotides in length, about 15 to about 35 nucleotides in length, about 18 to about 32 nucleotides in length, about 18 to about 30 nucleotides in length, or about 20 to about 35 nucleotides length.

In some embodiments, the siRNA includes a nucleotide sequence of SEQ ID NO: 1 to SEQ ID NO:17 (e.g., one of the foregoing sequences), or a combination thereof. In some embodiments, the siRNA includes a nucleotide sequence of SEQ ID NO: 4, SEQ ID NO: 5, SEQ ID NO: 6, SEQ ID NO: 8 to SEQ ID NO: 17, or a combination thereof. In some embodiments, the siRNA includes a nucleotide sequence of SEQ ID NO: 4, SEQ ID NO: 5, SEQ ID NO: 6, SEQ ID NO: 8 to SEQ ID NO: 17, or a combination thereof. In some embodiments, the siRNA includes a nucleotide sequence of SEQ ID NO: 6, SEQ ID NO: 8 to SEQ ID NO: 17, or a combination thereof.

ii. Lipophilic Ligand

The lipophilic ligand is capable of binding albumin, and thus can instill in the conjugate the ability to bind albumin. The lipophilic ligand can include any lipophilic moiety suitable for binding into a fatty acid pocket of albumin. In some embodiments, the lipophilic ligand includes a lipid with a long hydrocarbon chain.

The lipid can include a C12-C22 hydrocarbon chain, such as a C12-C20 hydrocarbon chain, a C14-C22 hydrocarbon chain, a C16-C22 hydrocarbon chain, or a C16-C20 hydrocarbon chain. In some embodiments, the lipid includes a C18 hydrocarbon chain. The lipid can be saturated or unsaturated. In addition, the lipid may have a terminal end. The terminal end may include a functional group that may aid in binding. In some embodiments, the terminal end of the lipid includes an alkyl, carboxyl, hydroxyl, or amino. In some embodiments, the terminal end of the lipid includes an alkyl or carboxyl. In embodiments where there is more than one lipid, each lipid can include a different functional group at its terminal end or can include the same functional group. For example, one terminal end can include an alkyl and one terminal end can include a carboxyl, or both terminal ends can include, e.g., an alkyl. In some embodiments, the terminal end includes an alkyl. In some embodiments, the terminal end of the lipid does not include a hydroxyl or a carboxyl.

The lipophilic ligand can include more than one lipid. Having more than one lipid can allow for multivalency of the lipophilic ligand and the conjugate thereof. The lipophilic ligand can include at least 2 lipids, at least 3 lipids, at least 4 lipids, at least 5 lipids, at least 6 lipids, at least 7 lipids, or at least 8 lipids. In some embodiments, the lipophilic ligand includes less than 10 lipids, less than 9 lipids, less than 8 lipids, less than 7 lipids, less than 6 lipids, or less than 5 lipids. In some embodiments, the lipophilic ligand includes 1 to 10 lipids, such as 1 to 8 lipids, 1 to 6 lipids, 2 to 8 lipids, 2 to 6 lipids, 2 to 4 lipids, or 2 to 3 lipids.

The lipophilic ligand can be divalent. For example, the lipophilic ligand can include two independent lipids. In some embodiments, the lipophilic ligand incudes two independent lipids, each lipid including a C12-C22 hydrocarbon chain. In some embodiments, the lipophilic ligand includes two independent lipids, each lipid including a C18 hydrocarbon chain.

The lipophilic ligand and lipid(s) can be attached to the hydrophilic spacer. In some embodiments, the lipophilic ligand and lipid(s) are directly attached to the hydrophilic spacer. In some embodiments, the lipophilic ligand includes two individual lipids, each lipid bound to a separate, individual hydrophilic spacer which is bound to a separate branch of the branching molecule. In such embodiments, the lipids may be the same or different. For example, the lipids can both include a C18 hydrocarbon chain. Alternatively, in other embodiments, each lipid can include a hydrocarbon chain of varying length. In some embodiments, the lipophilic ligand includes two distinct types of lipids.

iii. Linker

The lipophilic ligand is attached to the siRNA through a linker. The arrangement and composition of the linker can provide the conjugate with advantageous properties, such as, but not limited to, binding to albumin, decreased propensity to self-assembly into micelles, and improved pharmacokinetics. The linker includes a branching molecule and a hydrophilic spacer. The linker can further include other types of spacers and/or linkers known within the art.

a. Branching Molecule

The branching molecule can be any suitable molecule that allows for branching of the conjugate, e.g., extending from the siRNA. Example branching molecules include, but are not limited to, a phosphoramidite (e.g., symmetrical branching CED phosphoramidite), a tri-valent splitter, or a tetra-valent splitter.

The branching molecule can be positioned between the siRNA and the hydrophilic spacer. Or in other words, the branching molecule can be attached to the siRNA and the hydrophilic spacer. The branching molecule can also be directly attached to the siRNA. In some embodiments, the branching molecule is directly attached to the siRNA and attached to the hydrophilic spacer. In some embodiments, the branching molecule is directly attached to the siRNA and directly attached to the hydrophilic spacer. The branching molecule can be directly attached, or conjugated, to the siRNA through a phosphorothioate bond, phosphodiester, or cleavable linker (e.g., deoxythymidine (dT), pH-cleavable bond such as ketal). Although, in some embodiments, there are no intervening moieties or molecules when directly attached, as will be appreciated by those skilled in the art, the placement of the branch point within the branching molecule may be adjusted based upon the structure of the branching molecule itself.

The branching molecule can have multiple independent branch points, each branch point having at least two independent branches. For example, the branching molecule can have at least 2 branch points, at least 3 branch points, at least 4 branch points, or at least 5 branch points. In some embodiments, the branching molecule has less than 7 branch points, less than 6 branch points, less than 5 branch points, or less than 4 branch points. In some embodiments, the branching molecule has 1 to 5 branch points, such as 1 to 4 branch points, 1 to 3 branch points, or 1 to 2 branch points.

Each branch point can have multiple, independent branches. For example, each branch point can have at least 2 branches, at least 3 branches, at least 4 branches, at least 5 branches, at least 6 branches, at least 7 branches, at least 8 branches, at least 9 branches, or at least 10 branches. In some embodiments, each branch point has less than 12 branches, less than 11 branches, less than 10 branches, less than 9 branches, less than 8 branches, less than 7 branches, less than 6 branches, less than 5 branches, or less than 4 branches. In some embodiments, each branch point has 2 to 12 branches, such as 2 to 10 branches, 2 to 8 branches, 2 to 6 branches, or 2 to 4 branches. In some embodiments, each branch point has 2 branches.

The branching molecule's positioning in the conjugate can play an important role in determining properties of the overall conjugate. For example, attaching the branching molecule to the siRNA, rather than to the lipophilic ligand, and having the hydrophilic spacer between the branching molecule and the lipophilic ligand unexpectedly provides improved properties. It is hypothesized, without wishing to be bound by a particular theory, that the positioning of the hydrophilic spacer after the branching molecule can provide additional flexibility and separation between the lipophilic ligand and lipid(s) thereof. The lipophilic ligand and lipid(s) thereof with this additional flexibility and separation show higher affinity for albumin. Additionally, and again without wishing to be bound by a particular theory, it is hypothesized that attaching the branching molecule to the siRNA can decrease the self-micellization of the conjugate, which in turn can allow the conjugate to remain more unimeric in solution, and thus can be more available for binding to albumin. In addition, this may aid in binding to the outer surface of a cell membrane, which can promote internalization. In contrast, when the hydrophilic spacer is positioned before the branching molecule the lipophilic ligand and lipid(s) thereof can be more closely spaced, which can cause the conjugate to self-assemble, e.g., into a micelle, thereby limiting the lipophilic ligand's ability to interact with albumin.

The branching molecule can be included in the conjugate as a way to introduce multifunctionality, such as multivalency, to the conjugate. For example, the branching molecule can be used to increase the valency of the lipophilic ligand and the conjugate. The branching molecule can have independent branches that are attached to independent lipids. In some embodiments, the branching molecule has 2 independent branches that are attached to 2 independent lipids. The same can be said if there are 3 independent branches, these individual branches can be attached to 3 independent lipids. However, in some embodiments, not every branch is attached to a lipid. For example, in some embodiments, the branching molecule can have 4 branches, where only 2 of the 4 branches are attached to a lipid. Varying combinations of branches and their attachment to lipids can be used for the disclosed conjugate.

b. Hydrophilic Spacer

The hydrophilic spacer can include any suitable hydrophilic compound for attaching the lipophilic ligand to the branching molecule. Examples of suitable hydrophilic compounds include, but are not limited to, ethylene glycol, zwitterionic linkers, peptoids (e.g., poly(sarcosine)), amino acids, poly(ethylene glycol) substitutes including: poly(glycerols), poly(oxazoline), poly(acrylamide), poly(N-acryloyl morpholine, poly(N,N-dimethyl acrylamide), poly(2-hydroxypropyl methacrylamide), poly(2-hydroxyethyl methacryalmide), and any other similar hydrophilic spacer molecule and/or polymer.

The hydrophilic spacer can be attached to the lipophilic ligand and the branching molecule. As mentioned above, the branching molecule can include a branching point having at least two independent branches. Each branch of the branching molecule can be attached to an individual hydrophilic spacer. In addition, each hydrophilic spacer can be individually attached to an individual lipid of the lipophilic ligand. In some embodiments, the hydrophilic spacer is attached to the lipophilic ligand, the branching molecule, or both through phosphorothioate bonds. In some embodiments, the hydrophilic spacer is attached to a lipid of the lipophilic ligand, the branching molecule, or both through phosphorothioate bonds.

The hydrophilic spacer can include at least one hydrophilic block. For example, the hydrophilic spacer can include 1 to 100 hydrophilic blocks, such as 1 to 50 hydrophilic blocks, 1 to 20 hydrophilic blocks, 1 to 18 hydrophilic blocks, 2 to 15 hydrophilic blocks, 3 to 10 hydrophilic blocks, 2 to 10 hydrophilic blocks, 1 to 15 hydrophilic blocks, 1 to 10 hydrophilic blocks, 2 to 8 hydrophilic blocks, 2 to 6 hydrophilic blocks, or 1 to 7 hydrophilic blocks. In some embodiments, the hydrophilic spacer includes 5 hydrophilic blocks. The hydrophilic blocks can be attached to each other, the branching molecule, and/or the lipophilic ligand. For example, the hydrophilic blocks can be attached to each other, the branching molecule, and/or the lipophilic ligand through phosphorothioate bonds, phosphodiester, or a cleavable linker (e.g., deoxythymidine (dT), pH-cleavable bond such as ketal). In some embodiments, each of the hydrophilic blocks are attached to each other through phosphorothioate linkages.

The hydrophilic block can include repeats of the hydrophilic compound. In some embodiments, the hydrophilic spacer includes 1 to 100 hydrophilic blocks (as described above), with each of the repeating blocks including 1 to 150 repeats of the hydrophilic compound, such as 1 to 100 repeats of the hydrophilic compound, 2 to 50 repeats of the hydrophilic compound, 1 to 45 repeats of the hydrophilic compound, 1 to 30 repeats of the hydrophilic compound, 2 to 20 repeats of the hydrophilic compound, or 2 to 10 repeats of the hydrophilic compound. In some embodiments, each hydrophilic block includes less than 150 repeats of the hydrophilic compound, less than 100 repeats of the hydrophilic compound, less than 75 repeats of the hydrophilic compound, less than 50 repeats of the hydrophilic compound, less than 45 repeats of the hydrophilic compound, less than 40 repeats of the hydrophilic compound, or less than 35 repeats of the hydrophilic compound. In some embodiments, each hydrophilic block includes greater than 2 repeats of the hydrophilic compound, greater than 3 repeats of the hydrophilic compound, greater than 4 repeats of the hydrophilic compound, greater than 5 repeats of the hydrophilic compound, greater than 6 repeats of the hydrophilic compound, greater than 7 repeats of the hydrophilic compound, or greater than 8 repeats of the hydrophilic compound.

In some embodiments, the hydrophilic spacer includes 1 to 10 hydrophilic blocks, with each block including 1 to 15 repeats of the hydrophilic compound. In some embodiments, the hydrophilic spacer includes 1 to 10 hydrophilic blocks, with each block including 1 to 10 repeats of the hydrophilic compound. In some embodiments, the hydrophilic spacer includes 1 to 6 hydrophilic blocks, with each block including 2 to 10 repeats of the hydrophilic compound. In some embodiments, the hydrophilic spacer includes 2 to 6 hydrophilic blocks, with each block including 3 to 8 repeats of the hydrophilic compound.

The hydrophilic compound can be included in different variations as part of the hydrophilic block. For example, the hydrophilic spacer can include 1 block including 150 repeats of the hydrophilic compound, 2 blocks each including 50 repeats of the hydrophilic compound, 5 blocks each including 6 repeats of the hydrophilic compound, 2 blocks-one block including 5 repeats of the hydrophilic compound and the other block including 10 repeats of the hydrophilic compound, or any combination of blocks and repeats as disclosed herein.

In some embodiments, the hydrophilic spacer includes a plurality of ethylene glycol repeats. For example, the hydrophilic spacer can include 1 to 150 ethylene glycol repeats, 1 to 120 ethylene glycol repeats, 1 to 100 ethylene glycol repeats, 1 to 90 ethylene glycol repeats, 1 to 80 ethylene glycol repeats, 1 to 70 ethylene glycol repeats, 1 to 60 ethylene glycol repeats, 1 to 50 ethylene glycol repeats, 1 to 40 ethylene glycol repeats, 1 to 30 ethylene glycol repeats, 2 to 150 ethylene glycol repeats, 3 to 150 ethylene glycol repeats, 4 to 150 ethylene glycol repeats, 5 to 150 ethylene glycol repeats, 6 to 150 ethylene glycol repeats, 7 to 150 ethylene glycol repeats, 8 to 150 ethylene glycol repeats, 9 to 150 ethylene glycol repeats, 10 to 150 ethylene glycol repeats, 10 to 140 ethylene glycol repeats, 10 to 130 ethylene glycol repeats, 10 to 120 ethylene glycol repeats, 10 to 110 ethylene glycol repeats, 10 to 100 ethylene glycol repeats, 10 to 90 ethylene glycol repeats, 10 to 80 ethylene glycol repeats, 10 to 70 ethylene glycol repeats, 10 to 60 ethylene glycol repeats, 6 to 60 ethylene glycol repeats, 6 to 40 ethylene glycol repeats, 8 to 35 ethylene glycol repeats, or 10 to 32 ethylene glycol repeats. The ethylene glycol repeats can be included as a hydrophilic block(s) in different variations as described above.

In some embodiments, the hydrophilic spacer includes 1 to 10 hexaethylene glycol blocks (e.g., blocks of six ethylene glycol repeats). In some embodiments, the hydrophilic spacer includes 1 to 5 hexaethylene glycol blocks. In some embodiments, the hexaethylene glycol blocks are directly attached to each other and/or the branching molecule. In some embodiments, the hexaethylene glycol blocks are attached to each other and/or the branching molecule through phosphorothioate bonds, phosphodiester, or cleavable linker (e.g., deoxythymidine (dT), pH-cleavable bond such as ketal).

In some embodiments, the length of the hydrophilic spacer may be adjusted to provide desired properties. For example, ethylene glycol spacers with 18 ethylene glycol repeats can provide a higher binding to albumin. Alternatively, shorter spacers can yield conjugates with increased hydrophobicity, which can result in more tendency to bind lipoprotein complexes in the blood.

iv. Synthesis of Conjugates

Also provided herein are methods of synthesizing the conjugates. In some embodiments, the method includes solid phase synthesis where the full molecule is made/grown from a solid support, as opposed to solution phase conjugation of the siRNA to the linkers/lipidic moieties post-solid phase synthesis. For example, the branching molecule can be integrated during the solid phase synthesis. The integration of the branching molecule can convert the linear growth to divalent growth (or any number of valency), where the hydrophilic spacers can be added to the two growing chains following the branch point.

Further discussion of the conjugates for the disclosed methods can be found in International Patent Application Publication No. WO 2023/034561, which is incorporated by reference herein in its entirety.

v. Example Conjugates

In some embodiments, the conjugate includes a lipophilic ligand capable of binding albumin, the lipophilic ligand including two independent lipids, each lipid including a C18 hydrocarbon chain; and a linker attaching the siRNA to the lipophilic ligand, the linker including a branching molecule attached to the siRNA and including at least one branch point having at least two independent branches, and a hydrophilic spacer attaching an individual branch to an individual lipid, the hydrophilic spacer including 1 to 6 hydrophilic blocks, each hydrophilic block including 2 to 10 repeats of ethylene glycol.

### B. Administration

While siRNA-based therapeutics can be limited by rapid renal clearance, nuclease degradation, and inability to target/penetrate cells of interest, the conjugates disclosed herein can provide improved circulation half-life, can shield the siRNA from nucleases, and/or can provide extrahepatic delivery of the siRNAs. In addition, these advantages can be done without an associated carrier composition, such as a polymer or lipid formulation. Accordingly, in some embodiments the conjugate or pharmaceutical composition thereof is administered without an associated carrier composition.

The conjugate can be administered optionally in combination with a pharmaceutically acceptable excipient. Embodiments that administer the conjugate and a pharmaceutically acceptable excipient in combination can also be referred to as a pharmaceutical composition. Examples of pharmaceutically acceptable excipients include, but are not limited to, buffering agents (e.g., phosphate buffered saline, artificial cerebrospinal fluid (aCSF), etc.), carbohydrates (e.g., glucose, trehalose, starch, etc.) solubilizers, solvents, antimicrobial preservatives, antioxidants, suspension agents, penetration/absorption enhancers (e.g., DMSO, ethanol, pyrrolidones, and/or ionic liquids) or a combination thereof. In some embodiments, the pharmaceutically acceptable excipient includes saline, phosphate buffered saline (PBS), albumin, dimethyl sulfoxide, trehalose, sucrose, polyethylene glycol (PEG), an absorption enhancer, or a combination thereof. In some embodiments, the conjugate or pharmaceutical composition thereof does not include a carrier composition, such as a polymer- or lipid-based formulation. In some embodiments, the conjugate is administered in combination with a pharmaceutically acceptable excipient.

The conjugate or pharmaceutical composition thereof can be administered prophylactically or therapeutically. In prophylactic administration, the conjugate can be administered in an amount sufficient to induce a response. In therapeutic applications, the conjugate can be administered to a subject in need thereof in an amount sufficient to elicit a therapeutic effect. An amount adequate to accomplish this can be referred to as a “therapeutically effective dose.” Amounts effective for this use will depend on, e.g., the particular composition of the conjugate regimen administered, the manner of administration, the stage and severity of the disease, the general state of health of the patient, and the judgment of the prescribing physician.

The conjugate can be delivered via a variety of routes to the subject. Example delivery routes include, but are not limited to, intravenously, intracerebroventricularly, intrathecally, subcutaneously, and intra-cisterna magna. In some embodiments, the conjugate is administered intravenously. In some embodiments, the conjugate is administered intracerebroventricularly. In some embodiments, the conjugate is administered intrathecally. Following administration, the conjugate can bind albumin. In some embodiments, the conjugate is pre-complexed with albumin prior to administration.

The conjugate or pharmaceutical compositions thereof can be administered at varying suitable dosages, which can depend on a number of different factors, such as state of the CNS disease, progression of the CNS disease, age of the subject, route of administration, and other factors that would be recognized by the skilled artisan. In general, however, a suitable dose will often be in the range of about 0.01 mg/kg to about 1000 mg/kg, such as about 0.1 mg/kg to about 100 mg/kg, about 0.5 mg/kg to about 500 mg/kg, about 0.5 mg/kg to about 250 mg/kg, about 1 mg/kg to about 200 mg/kg, about 1 mg/kg to about 100 mg/kg, about 0.1 mg/kg to about 50 mg/kg, about 1 mg/kg to about 50 mg/kg, or about 0.1 mg/kg to about 60 mg/kg. Useful dosages of the conjugate can be determined by comparing their in vitro activity and in vivo activity in animal models thereof. Methods for the extrapolation of effective dosages in rodents, pigs, and other animals, to humans are known in the art; for example, see U.S. Pat. No. 4,938,949, which is incorporated by reference herein in its entirety.

The conjugate or pharmaceutical composition thereof can be administered at varying times and frequency. For example, the conjugate or pharmaceutical composition thereof can be administered at least once (e.g., as a single dose) over 100 days, such as 90 days, 80 days, 70 days, 60 days, 50 days, 40 days, 30 days, 25 days, 20 days, 15 days, 10 days, 5 days, or 1 day. In addition, the conjugate or pharmaceutical composition thereof can be administered as multiple doses over a period of time. For example, the conjugate or pharmaceutical composition thereof can be administered 1×, 2×, 3×, 4×, 5×, or more over 100 days—or a period of time listed above. And, the conjugate or pharmaceutical composition thereof may be administered as a single dose or as divided doses administered at appropriate intervals, for example, as two, three, four or more sub-doses per day. The sub-dose itself may be further divided, e.g., into a number of discrete loosely spaced administrations.

Suitable in vivo dosage to be administered and the particular mode of administration can vary depending upon the age, weight, the severity of the affliction, and subjects treated, the particular compounds employed, and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies.

Dosage amount and interval may be adjusted individually to provide plasma levels of the biologically active agent which are sufficient to maintain the modulating effects, or minimal effective concentration (MEC). The MEC will vary for each agent but can be estimated from in vivo and/or in vitro data. Dosages necessary to achieve the MEC will depend on individual characteristics and route of administration. However, assays well known to those in the art can be used to determine plasma concentrations. Dosage intervals can also be determined using MEC value. Conjugates or pharmaceutical compositions thereof can be administered using a regimen which maintains plasma levels above the MEC for 10-90% of the time, such as between 30-90% or between 50-90%. In cases of local administration or selective uptake, the effective local concentration of the conjugate may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest can vary with the severity of the symptoms to be treated and the route of administration. Further, the dose, and perhaps dose frequency, can also vary according to the age, body weight, and response of the individual patient. A program comparable to that discussed above may be used in veterinary medicine.

The conjugates or pharmaceutical compositions thereof described herein may be administered with additional compositions to prolong stability, delivery, and/or activity of the conjugate or pharmaceutical composition thereof, or combined with additional therapeutic agents, or provided before or after the administration of additional therapeutic agents.

Further discussion on the administration of the conjugates for the disclosed methods can be found in U.S. patent application Ser. No. 18/177,068, which is incorporated by reference herein in its entirety.

The disclosed technology has multiple aspects, illustrated by the following non-limiting examples.

### 3. Examples

### Materials & Methods for Examples 1-8

Reagents. 2′-O-Me and 2′-F phosphoramidites, universal synthesis columns (MM1-2500-1), and all ancillary RNA synthesis reagents were purchased from Bioautomation. Symmetrical branching CED phosphoramidite was obtained from ChemGenes (CLP-5215). Cyanine 5 phosphoramidite (10-5915), stearyl phosphoramidite (10-1979), biotin TEG phosphoramidite (10-1955), hexaethyleneglycol phosphoramidite (10-1918), TEG cholesterol phosphoramidite (10-1976), 5′-Amino-Modifier 5 (10-1905), and desalting columns (60-5010) were all purchased from Glen Research. All other reagents were purchased from Sigma-Aldrich unless otherwise specified.

Conjugate Synthesis, Purification, and Validation. Oligonucleotides were synthesized using modified (2′-F and 2′-O-Me) phosphoramidites with standard protecting groups on a MerMade 12 Oligonucleotide Synthesizer (Bioautomation). Amidites were dissolved at 0.1M in anhydrous acetonitrile with the exception of 2′OMe U-CE phosphoramidite, which utilized 20% anhydrous dimethylformamide by volume as a cosolvent, and stearyl phosphoramidite, which was dissolved in 3:1 dichloromethane: acetonitrile by volume. Coupling was performed under standard conditions, and strands were grown on controlled pore glass with a universal terminus (1 μmol scale, 1000 Å pore size).

Strands were cleaved and deprotected using 1:1 methylamine: 40% ammonium hydroxide at room temperature for 2 hours. Lipophilic RNAs were purified by reversed-phase high performance liquid chromatography using a Clarity Oligo-RP column (Phenomenex) under a linear gradient from 85% mobile phase A (50 mM triethylammonium acetate in water) to 100% mobile phase B (methanol) or 95% mobile phase A to 100% mobile phase B (acetonitrile). Oligonucleotide containing fractions were then dried using a Savant SpeedVac SPD 120 Vacuum Concentrator (ThermoFisher). Conjugates were then resuspended in nuclease free water and sterile filtered before lyophilization.

Conjugate molecular weight and purity was confirmed using Liquid Chromatography-Mass Spectrometry (LC-MS) analysis on a ThermoFisher LTQ Orbitrap XL Linear Ion Trap Mass Spectrometer. Chromatography was performed using a Waters XBridge Oligonucleotide BEH C18 Column under a linear gradient from 85% A (16.3 mM triethylamine-400 mM hexafluoroisopropanol) to 100% B (methanol) at 45° C. Control conjugate, si-EG₄₅L₂, molecular weight was validated using MALDI-TOF mass spectrometry using 50 mg/mL 3-hydroxypicolinic acid in 50% water, 50% acetonitrile with 5 mg/mL ammonium citrate as a matrix.

Synthesis of amine-reactive lipids and subsequent modification of oligonucleotides was adapted from methods reported by Prakash, T. P. et al. Fatty acid conjugation enhances potency of antisense oligonucleotides in muscle. Nucleic Acids Res 47, 6029-6044 (2019), which is incorporated herein by reference in its entirety. Briefly, amine-terminated oligonucleotides were speed vacuumed to dryness and desalted to remove MMT groups. Oligonucleotides were then lyophilized followed by reconstitution in 0.1 sodium tetraborate (pH 8.5) to a concentration of 500 μM. PFP-modified lipid was dissolved into a mixture of acetonitrile, DMSO, and triethylamine (70:29:1 by volume) at a concentration of 7 μM. Aqueous oligonucleotide was added dropwise to the organic solution for a 1:40 molar ratio of oligonucleotide-amine:amine-reactive lipid (approximately 25% 0.1M sodium tetraborate, 75% organic mixture). Solution was stirred overnight and desalted prior to purification and characterized.

Purified oligonucleotide was resuspended in 0.9% sterile saline and annealed to its complementary strand by heating to 95° C. and cooling stepwise by 15° C. every 9 min until 25° C. on a T100 Thermal Cycler (BioRad).

Duplexes directly bound to albumin were synthesized in a two-step, one-pot reaction. Briefly, conjugate covalently bound to albumin was synthesized by first reacting azido-PEG₃-maleimide (Click Chemistry Tools) with the free thiols on human (1 free SH) or mouse (2 free SH). Albumin was dissolved in PBS with 0.5M EDTA to a final concentration of 10 mM. Anhydrous DMF was used to solubilize and activate azido-PEG₃-maleimide. DBCO-modified siRNA duplex in PBS was reacted at a 1:1 ratio of DBCO groups:free SH groups and allowed to incubate at room temperature for 4 hours. To remove any siRNA that did not react with albumin, or reacted only with the azido linker, the resulting solution underwent 10 rounds of centrifugation in a 30 kDa cutoff Amicon filter at 14,000×g for 10 minutes for each round. Conjugation was confirmed by gel electrophoresis of precursor DBCO-siRNA alongside resulting siRNA-DBCO-albumin.

Cell Culture. Cells were cultured in Dulbecco's modified eagle's medium (DMEM, Gibco), containing 4.5 g/L glucose, 10% FBS (Gibco), and 50 μg/mL gentamicin. All cells were tested for Mycoplasma contamination MycoAlert Myocplasma Detection Kit (Lonza).

In Vitro Knockdown Experiments. For lipofection-mediated knockdown experiments, luciferase-expressing MDA-MB-231s were seeded at 4,000 cells per well in 96 well plates in complete media. After 24 h, cells were treated with siRNA (25 nM) using Lipofectamine 2000 (ThermoFisher) in OptiMEM according to manufacturer protocol, replacing with complete media at 24 h post-transfection, and measuring luciferase activity at 48 h post-transfection in cells treated for 5 min with 150 μg/mL D-Luciferin, potassium salt (ThermoFisher) using an IVIS Lumina III imaging system (Caliper Life Sciences).

Serum Stability. siRNA (0.1 nmol) in 60% fetal bovine serum in PBS was incubated at 37° for 0-48 h, then assessed on a 2% agarose gel in 1×TAE Buffer. Gels were stained with GelRed Nucleic Acid Stain (Biotium) according to the manufacturer's protocol.

Biolayer Interferometry. Binding kinetics were measured by biolayer interferometry using an Octet RED 96 system (ForteBio). Duplexes synthesized with TEG-Biotin on the 5′ terminus of the antisense strand were diluted to 500 nM in Dulbecco's phosphate buffered saline containing calcium and magnesium (DPBS^{+/+}) and loaded on a Streptavidin Dip and Read Biosensor (ForteBio) for 600 sec. Baseline was then established over 120 sec in DPBS^{+/+} followed by association to either human or mouse serum albumin in DPBS^{+/+} over 300 sec. Subsequently, the biosensor was immersed in DPBS^{+/+} for 300 sec to measure dissociation. All steps were conducted at 30° C. and 1000 rpm. The binding values were measured using Octet Data Analysis HT Software. Reference biosensor values (biotinylated conjugate bound with no analyte) were subtracted to account for signal background. Y axes were aligned to the average of the baseline step. Interstep correction was performed by aligning to the dissociation step, and noise filtering was performed. Global analysis was performed to derive constants simultaneously from all tested analyte concentrations.

Critical Micelle Concentration. A serial dilution of duplexes was prepared in a 96-well plate from 20 μM to 10 nM in 50 μL of DPBS (Ca²⁺/Mg²⁺ free). Nile Red (1 μL of a 0.5 mg/mL stock solution) was added to each well. Samples were then incubated in the dark with agitation at 37° C. for 2 h, and fluorescent intensity was measured on a plate reader (Tecan) at excitation 535±10 nm and emission 612±10 nm. The critical micelle concentration was defined as the intersection point on the plot of the two linear regions of the Nile red fluorescence versus the duplex concentration.

Gel Migration Shift Experiments. Binding of siRNA conjugates (0.1 nmol) to human or mouse serum albumin (in 5× molar excess) incubated for 30 min at 37° C. was assessed by migration through 4%-20% polyacrylamide gels (Mini-Protean TGX). siRNA was visualized with GelRed Nucleic Acid Stain (Biotium) for ultraviolet imaging, and proteins were visualized with Coomassie blue and visible light imaging.

Conjugation efficacy of DBCO-modified siRNA duplex with azide-modified albumin was visualized using the Agilent Protein 230 Assay on the Agilent 2100 Bioanalyzer according to manufacturer instructions.

Long-Term Fluorescence of Blood Samples. Longer term pharmacokinetic profiles of siRNA conjugates were established by measuring fluorescence of blood samples taken at various time points from 5 min to 24 h. Blood was sampled in the contralateral vein from injection (˜10 μL) using EDTA coated capillary tubes and stored at −80° C. Blood was then thawed and diluted 40× with phosphate buffered saline in a 96-well plate and fluorescent intensity was measured.

Intravital Microscopy and Biodistribution. Microscopy was performed using a Nikon Czsi+ system. Isoflurane-anesthetized, 6-8 week old male CD-1 mice (Charles River) were immobilized on a heated confocal microscope stage for ear vein imaging. Mouse ears were depilated and then immobilized on a glass coverslip using microscope immersion fluid. Ear veins were detected using light microscopy, and images were focused to the plane of greatest vessel width, where flowing red blood cells were clearly visible. Once in focus, confocal laser microscopy was used to acquire one image per second, at which point Cy5-labeled siRNA (1 mg/kg) in 100 μL was delivered via tail vein. Fluorescent intensity within a circular region of interest (ROI), drawn in the focused vein, was used to measure fluorescence decay. Values are normalized to maximum initial fluorescence and fit to a one-compartment model in PK Solver to determine pharmacokinetic parameters.

Approximately 45 min after delivery of Cy5-labeled siRNA, blood was collected by cardiac puncture using EDTA-coated tubes and used for plasma isolation. Cy5 fluorescence was quantified in heart, lung, liver, kidney, and spleen using IVIS Lumina Imaging system (Xenogen Corporation) at excitation and emission wavelengths of 620 and 670 nm, respectively, using Living Image software version 4.4.

Size Exclusion Chromatography (SEC). Murine plasma was filtered (0.22 μm) then injected into an AKTA Pure Chromatography System (Cytiva) with three inline Superdex 200 Increase columns (10/300 GL) for fractionation at 0.3 mL/min using Tris running buffer (10 mM Tris-HCl, 0.15M NaCl, 0.2% NaN₃) into 1.5 mL fractions with a F9-C 96-well plate fraction collector (Cytiva). Cy5 fluorescence was measured in fractions (100 μL) in black, clear-bottom, 96-well plates (Greiner-Bio-one REF 675096) on a SynergyMx (Biotek) at a gain of 120, excitation 642/9.0, emission 675/9.0. Fraction albumin-bound conjugate was determined by taking the sum of fluorescence intensity for fractions associated with albumin elution divided by the sum of fluorescence intensity for all fractions collected. Albumin-associated fractions were determined by running known protein standards through the SEC system and examining A280 of eluent from each of the fractions.

Statistical Analyses. Data were analyzed using GraphPad Prism 7 software (Graphpad Software, Inc.) Statistical tests used for each data are provided in the corresponding figure captions. For all figures, * p≤0.05 ** p≤0.01 *** p≤0.001 **** p≤0.0001. All plots show mean±standard deviation.

### Example 1

Divalent Lipid Modifier Improves Bioavailability of Chemically Stabilized siRNAs

To finely tune and examine the structure-function relationship of siRNA variants, a library of siRNA-lipid conjugates was generated using solid phase synthesis, which maximizes product yield, purity, and reproducibility compared to previously reported two-step solution phase conjugation as described in Sarett, S. M. et al. Lipophilic siRNA targets albumin in situ and promotes bioavailability, tumor penetration, and carrier-free gene silencing. Proceedings of the National Academy of Sciences of the United States of America 114, E6490-E6497 (2017), which is incorporated herein by reference in its entirety. The synthesized siRNAs were designed to be fully stabilized with alternating 2′F and 2′OMe modifications in a “zipper” pattern and terminal phosphorothioate linkages. These modifications can confer endonuclease and exonuclease resistance. It was demonstrated that these stabilizing siRNA modifications maintain gene silencing potency and provide serum stability, while traditional Dicer substrate siRNAs are similarly potent but degrade within 4 h of serum challenge.

Valency may affect bioavailability and pharmacodynamics of lipid end-modified siRNA conjugates in vivo. Conjugation to one or two 18-carbon stearyls was focused on, an albumin-binding lipid with higher albumin affinity than those with shorter lipid chain lengths, for initial assessment of modifier valency on siRNA pharmacokinetics. Absolute circulation half-life (t_{1/2}) was measured using real-time fluorescence imaging of Cy5-labeled siRNA conjugates within mouse vasculature, revealing increased t_{1/2} (46±5.9 min) of siRNA conjugated to divalent (L₂) over monovalent (L₁) stearyl (28±4.2 min). Importantly, L₂-conjugated siRNA (siRNA-L₂) showed diminished kidney accumulation compared to siRNA-L1, suggesting that renal clearance, the primary elimination mechanism of circulating siRNAs, is reduced with siRNA-L₂. The remainder of the studies, therefore, focused on a divalent lipid design.

### Example 2

### Hydrophilic Linker Length Increases In Vitro Albumin Affinity

Based on the improved performance of siRNA-L₂ over siRNA-L1 in vivo, siRNA-L₂ was modified to assess the functional effects of structural modification of the hydrophilic linker between the lipid and siRNAs. Specifically, the number of ethylene glycol (EG) repeats were progressively increased from no EG repeats [si<(EG⁰L)₂] to 30 EG repeats [si<(EG₃₀L)₂]; the EG repeats were added in increments of 6, using a hexaethylene phosphoramidite. Two previously described serum protein-binding siRNA conjugates, cholesterol-TEG-siRNA (si-chol) and si-EG₄₅<L₂, were synthesized as comparative references.

Each siRNA<(EGₓL)₂ was incubated with human serum albumin to assess albumin-siRNA complex formation by electrophoretic mobility shift assay. These studies revealed that, while electrophoretic mobility of free siRNA was unaffected by albumin, the mobility si<(EG₀L)₂, si<(EG₆L)₂, si<(EG₁₈L)₂, and si<(EG₃₀L)₂ was lowered upon exposure to albumin, consistent with the high molecular weight of the complex formed by albumin and siRNA conjugates. Similarly, si-cholesterol, and si-EG₄₅<L₂ also displayed albumin-dependent mobility shifts in this assay. However, super-shifting of si-EG₄₅<L₂ was seen in both the presence and absence of albumin, suggesting that si-EG₄₅<L₂ may harbor some self-association properties which were not seen in si<(EGₓL)₂ conjugates. Similar results were observed using mouse serum albumin.

Albumin association and dissociation kinetics of the si<(EGₓL)₂ variants were studied further using biolayer interferometry. Free siRNA did not exhibit binding with albumin, while si-cholesterol exhibited moderate albumin binding. Although si<(EG₀L)₂ displayed decreased HSA binding response compared to si-cholesterol, siRNA conjugates harboring a greater number of EG spacers within the linker element had progressively higher affinity for HSA, with both Si<(EG₁₈L)₂ (K_{D}=30±0.3 nM) and si<(EG₃₀L)₂ (K_{D}=9.49±0.1 nM) exhibiting higher affinity albumin binding than si-cholesterol and si-EG₄₅<L₂. The substantial difference in albumin binding response between variants underscores the role of the EG repeats within the linker region.

Amphiphilic lipid-modified nucleic acids have a tendency to self-assemble into micellar structures, particularly when using long lipid chains. It is possible that self-aggregation of amphiphilic siRNA-lipid conjugates is a competing interaction that might interfere with albumin association, particularly if lipid tails can become sequestered in the core of a self-assembled structure, where they would be rendered unavailable for interaction with the fatty acid binding pockets of albumin. Thus, the critical micelle concentration (CMC) was determined for each si<(EGₓL)₂ to establish the impact of linker length on siRNA conjugate self-assembly. The si-cholesterol exhibited a relatively high CMC (3430±350 nM), suggesting a low tendency for si-cholesterol to self-associate. This is consistent with the bulky structure of cholesterol, which is not amenable to close packing like lamellar long-chain lipids. In contrast, si-EG₄₅<L₂ exhibited a lower CMC (1860±60 nM), indicating a higher tendency towards self-association. Interestingly, si<(EG₀L)₂, which lacks any EG spacer in the linker element, exhibited the lowest CMC (1040±23 nM) and thus the highest propensity for self-association, while the increased number of EG repeats in si<(EG₁₈L)₂ and si<(EG₃₀L)₂ correlated with the highest CMCs (3260±190 nM and 3330±210 nM), and thus the lowest tendency towards self-association.

### Example 3

### Hydrophilic Linker Length Influences Pharmacokinetics and In Vivo Plasma Disposition of Lipid-siRNA Conjugates

The structure of hydrophobic modifications on siRNA can be tuned to direct siRNA binding to different serum components, such as lipoproteins and albumin, after intravenous administration, which can consequently modify pharmacokinetics and biodistribution. The in vivo half-life of human albumin is approximately 19 days, making it a good candidate for improving the pharmacokinetics of candidate therapeutics.

Each of the siRNA conjugates was intravenously administered to mice to understand how EG repeats within the linker affect conjugate pharmacokinetics. Intravital fluorescence microscopy of Cy5-labeled siRNA conjugates flowing through vessels of the mouse ear demonstrated the rapid and complete diminution of circulating free siRNA within the first 30 min post-treatment, while serum component-binding si-cholesterol and si-EG₄₅<L₂ retained some observable circulating siRNA. Interestingly and unexpectedly, increased EG repeats correlated with increased retention of circulating siRNA in si<(EG_{X}L)₂ conjugates to a point, but once the linker became too long (e.g., si<(EG₃₀L)₂), retention was reduced, with si<(EG₁₈L)₂ showing maximal circulation retention of the variants tested. Real-time collection of vascular fluorescence imaging data throughout the first hour post-treatment enabled calculation of absolute half-life (t_{1/2 abs}), demonstrating the substantially prolonged t_{1/2 abs} of si<(EG₁₈L)₂, which was greater than what was observed for si<(EG₃₀L)₂, and nearly 5 times that of si<(EG₀L)₂ (Table 1). Approximately 45 min after injection, there was significantly more renal accumulation, the primary clearance path for oligonucleotide-based therapeutics, of the parent siRNA and control conjugate, si-EG₄₅<L₂.

TABLE 1
Pharmacokinetic parameters for siRNA conjugate
library determined from intravital microscopy.

| | | t_{1/2} | AUC₀₋₃₅ₘ | CL |
|---|---|---|---|---|
| | | (min) | (μ · min)/(mL) | (mL)/(min) |
| EG | siRNA | 14 ± 3.5 | 210 ± 25 | 0.069 ± |
| Variants | | | | 0.012 |
| | si-EG₃-cholesterol | 28 ± 12 | 410 ± 42 | 0.025 ± |
| | | | | 0.0079 |
| | si-EG₄₅ < L₂ | 27 ± 5.3 | 390 ± 25 | 0.026 ± |
| | | | | 0.0046 |
| | si < (EG₀L)₂ | 33 ± 6.6 | 416 ± 55 | 0.028 ± |
| | | | | 0.0059 |
| | si < (EG₆L) ₂ | 36 ± 4.6 | 450 ± 15 | 0.019 ± |
| | | | | 0.0022 |
| | si < (EG₁₈L) ₂ | 64 ± 23 | 470 ± 52 | 0.012 ± |
| | | | | 0.0032 |
| | si < (EG₃₀L) ₂ | 37 ± 6.6 | 440 ± 36 | 0.019 ± |
| | | | | 0.0043 |
| PS | si < (EG₁₈L)₂No 5′Se | 37 ± 12 | 430 ± 50 | 0.020 ± |
| Variants | PS | | | 0.0060 |
| | si < (EG₁₈L)₂No 5′Se | 15 ± 1.5 | 300 ± 36 | 0.047 ± |
| | or Binder PS | | | 0.0070 |
| Branching | si-(EG₁₈) < L₂ | 67 ± 32 | 490 ± 35 | 0.012 ± |
| Variants | | | | 0.0070 |
| | si-(EG₃₆) < L₂ | 41 ± 13 | 450 ± 27 | 0.018 ± |
| | | | | 0.0050 |
| Lipid | si < (EG₁₈L_{diacid})₂ | 47 ± 21 | 469 ± 64 | 0.017 ± |
| Variants | | | | 0.0073 |
| | si < (EG₁₈L _{unsaturated}) ₂ | 34 ± 26 | 391 ± 121 | 0.029 ± |
| | | | | 0.016 |

It is hypothesized, without being bound by a particular theory, that the diminished renal clearance of the si<(EG_{X}L)₂ variants over si-EG₄₅<L₂ is due to the presence of hydrolytically degradable ester bonds located in the structure of DSPE-PEG₂₀₀₀ used to make si-EG₄₅<L₂. Albumin itself is known to possess intrinsic esterase activity, making the hydrolytic stability of drugs that interact with it particularly important. Hydrolysis of this ester would be expected to release the siRNA from albumin, possibly prematurely in the circulation, resulting in renal clearance and shorter circulation time that is more analogous to the non-modified parent siRNA structure. Conjugates that remain albumin-bound, by comparison, can evade renal clearance through albumin's natural reabsorption in the renal proximal tubule where, after endocytosis by the megalin-cubilin complex, the neonatal Fc receptor redirects albumin and its associated cargo back to the interstitial space, facilitating its return to the circulation via the lymphatics.

Plasma was collected from mice treated intravenously with Cy5-labeled siRNA conjugates and analyzed by size exclusion chromatography to measure the level of each candidate's association with albumin versus other plasma fractions (e.g., lipoproteins). Cy5 fluorescence was detected in albumin-containing plasma fractions, as well as in fractions not representative of albumin elution. Notably, plasma isolated from mice treated with si<(EG₁₈L)₂, the most long-circulating on the investigated conjugates, exhibited the most robust peak within the albumin-containing fraction at approximately 75% bound. This observation is consistent with the BLI data showing the high affinity of si<(EG₁₈L)₂ with albumin and also suggests a positive correlation between the percent of conjugate that is albumin-bound in vivo and the circulation half-life.

It is interesting and unexpected that in vitro albumin binding affinity analyses indicate that si<(EG₃₀L)₂ binds albumin more favorably than si<(EG₁₈L)₂, while albumin association in vivo is greater with si<(EG₁₈L)₂. This may be attributable to the larger entropic penalty of binding incurred for this larger and more flexible molecule that is mitigated by the ideal conditions of in vitro testing but becomes more apparent in vivo.

### Example 4

### Phosphorothioate Linkages of Lipid-Modified Terminus Improves Conjugate Performance In Vitro and In Vivo

Deeper structural interrogation of si<(EG₁₈L)₂ was next focused on by assessing the impact of the phosphorothioate (PS) bonds at the 5′ sense terminus and between the EG₆ repeating units of the linker. Terminus stabilization with PS linkages in lieu of phosphodiester (PO) linkages has significant effects on performance of siRNA-based therapeutics by conferring exonuclease resistance and can help enable extrahepatic, carrier-free gene silencing applications. Variants of si<(EG₁₈L)₂ with PS bonds removed from the 5′ sense (Se) terminus (si<(EG₁₈L)₂ No 5′Se PS) or removed from both the 5′ sense terminus and each of the bonds in the linker to the stearyl groups (si<(EG₁₈L)₂ No 5′Se or Binder PS) were synthesized and studied using biolayer interferometry to determine albumin binding kinetics. Both variants exhibited comparable albumin affinity as the parent construct with K_{D} values only varying ±2 nM. However, removing the PS bonds from the linker significantly increased the critical micelle concentration compared to just removing it from the 5′ sense terminus (2755±526 to 3798±225 nM), suggesting a lower tendency to self-assemble without the more hydrophobic PS bonds located on the linker.

Real-time, intravital microscopy of fluorescently labeled conjugates in circulation was performed to determine the effect of PS content on circulation time. Removal of PS bonds from both the 5′ sense terminus and the 5′ sense terminus/binder resulted in significantly diminished pharmacokinetic profiles compared to parent construct si<(EG₁₈L)₂ with circulation half-lives reduced to 37±12 min and 15±1.5 min respectively from 64±23 min. Biodistribution of conjugates approximately 45 min after treatment demonstrated significant differences in PS-dependent accumulation in both the lungs and the liver. Plasma collected from mice treated intravenously with each siRNA conjugate to examine proteins bound in vivo demonstrated 10-fold lower relative albumin binding by si<(EG₁₈L)₂ siRNA conjugate when the PS bonds were not used at the 5′ sense terminus. These combined data suggest that the PS bonds within the linker facilitate albumin binding. The diminished pharmacokinetics observed with the removal of phosphorothioate bonds is most likely due to reduced albumin association and not due to degradation. This conclusion is supported by the observation that, unlike ester-containing si-EG₄₅<L₂, the kidney accumulation of the conjugate without PS bonds was similar to the fully modified parent construct.

### Example 5

### Position of Branching Point in Divalent Lipid Conjugate Affects Conjugate Performance In Vitro and In Vivo

It was sought to determine whether the position of the branching point in the divalent si<(EG₁₈L)₂ conjugate influences candidate function. This study was motivated by the desire to further confirm the observation that albumin association is driven by reduced tendency to self-assemble rather than simply the relative level of conjugate hydrophilic linker content or linker length. It is hypothesized, without being bound by a particular theory, that placement of the branching point distal to the repeating EG linker and immediately proximal to the stearyl groups increases apparent hydrophobicity and consequent self-assembly by constraining the stearyl groups to remain tightly packed together. Since the greatest siRNA circulation time was seen with the si<(EG₁₈L)₂ conjugate, which correlated with increased albumin binding in vivo, two additional iterations of si<(EG₁₈L)₂ were generated with a distal branching location, one matched for overall ethylene glycol content (si-EG₃₆<L₂) and one matched for the distance between the siRNA and its lipid tail (si-EG₁₈<L₂). Biolayer interferometry measurement of albumin binding in vitro interestingly showed that both new branching point variants had lower albumin binding affinity relative to the parent construct si<(EG₁₈L)₂. The CMC was measured by Nile Red encapsulation and both si-EG₁₈<L₂ and si-EG₃₆<L₂, similarly to control construct si-EG₄₅<L₂, exhibited significantly lower values (1838±117 and 2293±132 nM) compared to si<(EG₁₈L)₂ (3255±192 nM). These data suggest that the more proximal branch site increases albumin association and consequent siRNA activity at least partially due to decreased tendency of amphiphilic siRNA conjugates with this feature to self-assemble.

Intravital microscopy of circulating fluorescent conjugates showed that there was not a significant difference in absolute circulation half-life among the branching architecture variants. However, biodistribution of the fluorescently labeled conjugates approximately 45 min after administration revealed significantly higher liver accumulation of si-EG₁₈<L₂ and si-EG₃₆<L₂ compared to si<(EG₁₈L)₂. This observation may be explained by a tendency to bind a greater fraction of lipoproteins over albumin in vivo which can preferentially traffic the conjugates to the liver. Indeed, plasma isolated from mice treated with the conjugates and analyzed by SEC for associated proteins demonstrated significantly decreased albumin binding among branching architecture variants si-EG₁₈<L₂ and si-EG₃₆<L₂ (˜5%) compared to si<(EG₁₈L)₂ (˜75%). In sum, these data show that the distal placement of the branching point increases self-assembly and association with lipoproteins at the cost of diminished association with and piggybacking upon serum albumin.

### Example 6

Hydrophobicity of Lipid is More Important than Binding Affinity for Conjugate Performance

It was next sought to investigate whether the nature of the C₁₈ lipid itself has important implications in conjugate performance. To this end, two variants were synthesized—one with the carboxyl terminal still intact (si<(EG₁₈L_{diacid})₂) and one with a double bond (si<(EG₁₈L_{unsaturated})₂). The carboxyl handle of fatty acids is usually consumed in conjugation reactions. However, the development of GLP-1 agonist drug Semaglutide demonstrated the importance of this moiety for albumin-binding drugs. Indeed, by restoring the carboxyl on their lipid-peptide, they found significant increases in albumin affinity and circulation half-life. Thus, it was sought to explore whether having this group in this system would improve performance.

Further, interest in testing a variant with a double bond was motivated by 1) evidence suggesting that oleate, an unsaturated variant of stearate, possesses a higher affinity for albumin and 2) the idea that double bonds introduce “kinks” into lipid chains that prevent close packing and may therefore deter self-assembly, which was shown to correlate with poorer albumin binding. To test these hypotheses, si<(EG₁₈-Amine)₂ was synthesized and conjugated amine-reactive PFP-modified lipid variants on to the terminus of the conjugates. Both the diacid and unsaturated variants of the conjugate exhibited comparable kidney accumulation and absolute circulation half-life. However, si<(EG₁₈L_{unsaturated})₂ demonstrated significantly diminished albumin binding in vivo compared to its saturated and diacid counterparts (˜40% bound versus ˜75-80% bound). Based on the correlations of albumin-bound in vivo observed previously, only the diacid variant was further characterized. Strikingly, si<(EG₁₈L_{diacid})₂ showed a substantially stronger binding response to albumin, with affinity for human serum albumin approximately two orders of magnitude superior to its non-acid counterpart (si<(EG₁₈L_{diacid})₂ KD=0.15±0.002 nM and si<(EG₁₈L)₂ KD=30±0.3 nM). To determine whether the impact of this increased affinity for albumin would be captured by circulation half-life at longer time points, blood was sampled from mice injected intravenously with 5 mg/kg of either fluorescently labeled si<(EG₁₈L)₂ or its diacid counterpart but found no significant difference in their PK profiles.

### Example 7

Albumin-Binding Lipophilic siRNA Conjugates Outperform SiRNA Directly Conjugated to Albumin

To further interrogate the appeal of an albumin-binding, lipophilic conjugate, an siRNA duplex was synthesized directly, covalently bound to mouse serum albumin. It was sought to determine whether the lipid-mediated, reversible binding was preferable to maximized albumin-bound delivery. These complexes were synthesized by leveraging the two free thiol groups present on mouse serum albumin as a handle for modifying with an azido-PEG₃-maleimide linker followed by reacting with DBCO-modified siRNA duplex. Gel electrophoresis demonstrated an upward shift of resulting product relative to the DBCO-duplex precursor, suggesting successful conjugation and removal of unreacted ligands. Fluorophore-labeled duplex was additionally used to validate that A260 readouts of product agreed with fluorescent readouts for quantification of siRNA in the resulting complex. Plasma isolated from mice injected with the siRNA-MSA complex demonstrated that approximately 80% of the siRNA was associated with fractions associated with albumin. Strikingly, however, the observed half-life of the siRNA covalently bound to albumin was greatly diminished compared to the lipophilic siRNA conjugate. Previous reports have shown that cell surface glycoproteins gp18 and gp30 can bind to covalently modified albumin and act as scavenger receptors that traffic the modified albumin for lysosomal degradation. This is further supported by the organ biodistribution of the siRNA taken from the same mice, which shows no significant difference in kidney levels between the groups. This suggests that liberation from the albumin resulting in renal clearance is not responsible for the reduction in circulation half-life.

The examples disclosed herein show that systematic variation of lipid-siRNA conjugate valency, linker length, phosphorothioate bonds, lipid chemistry, and linker branching architecture impacts albumin binding, pharmacokinetics, and tissue biodistribution. It is shown that lipid valency has a significant impact on pharmacokinetics. Further, the addition of a hydrophilic linker improves albumin binding, but not indiscriminately. The data suggest that there is an advantageous length of hydrophilic linker, e.g., [si<(EG₁₈L)₂], and that this linker improves albumin-binding when placed after the branching point of the divalent moiety. Notably, designs with the hydrophilic linker before the branching point of the divalent structure, when matched for both overall hydrophobicity or length between the siRNA and lipids, showed inferior albumin association in plasma in vivo, which may be attributable to greater lipoprotein association and propensity for self-assembly (lower CMC). The examples also showed that phosphorothioate, rather than phosphodiester, linkages are beneficial both within the linker structure and sense strand terminus where the albumin-binding moiety is located, possibly to promote plasma protein binding. It was additionally demonstrated that the increased albumin affinity conferred by keeping the carboxyl handle of the fatty acid intact is outcompeted by the need for hydrophobicity to achieve silencing efficacy. Further, the examples suggest that siRNA benefits from being reversibly, rather than covalently, bound to albumin.

Overall, this work has important implications for the delivery of siRNAs to extrahepatic targets, a goal that has remained clinically elusive. Albumin is known to accumulate at sites of inflammation and vascular leakiness. Therefore, the insights gleaned from the development of si<(EG₁₈L)₂ as a beneficial siRNA structure for in situ albumin piggybacking can have far reaching implications for the development and improvement of extrahepatic, carrier-free siRNA therapeutics.

### Example 8

### Toxicity Analysis of L2-siRNA

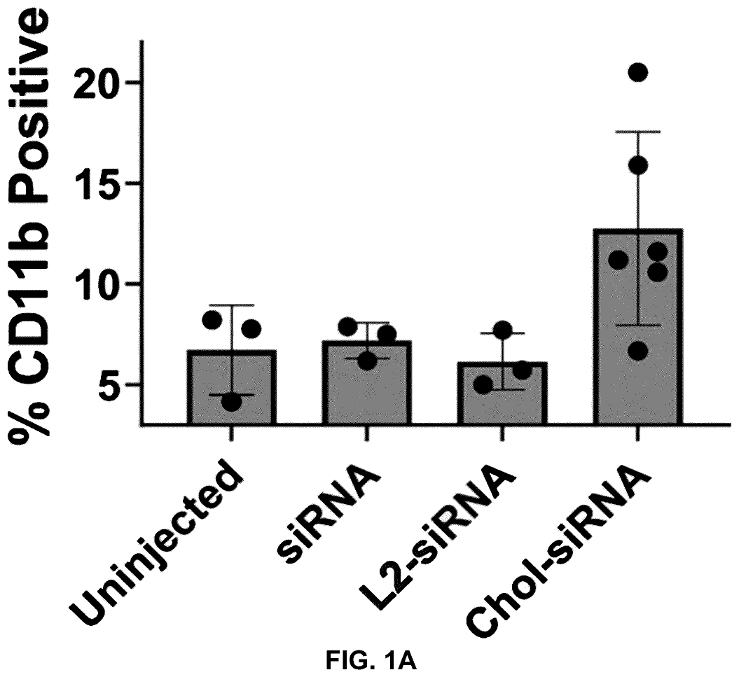

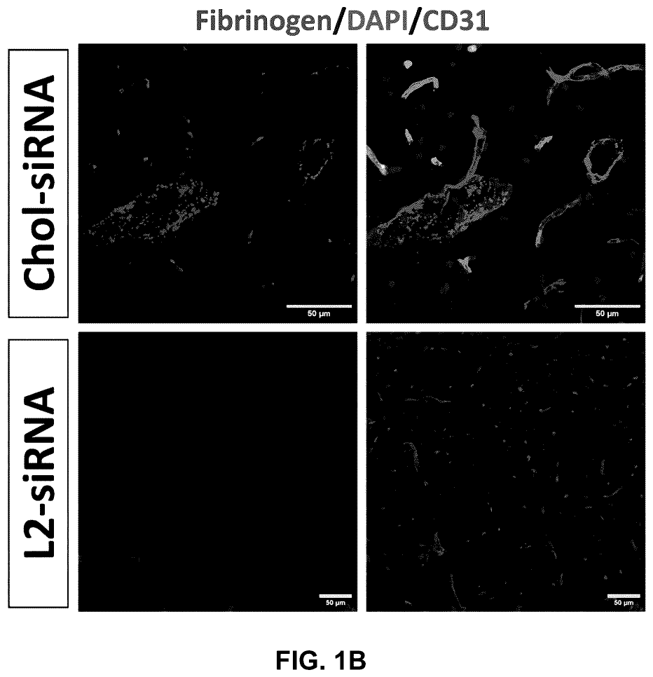

Since toxicity can be a concern for delivery into the CSF, the safety profile of EG18 (e.g., L2-siRNA) was examined relative to Chol. First, reactive microgliosis was assessed, a common feature of brain inflammation where microglia proliferate and undergo morphological changes. Microglia number were unchanged by free siRNA or siRNA-EG18 treatment, whereas microglia number doubled after Chol treatment (FIG. 1A). Further, to assess damage to blood-brain barrier, fibrinogen leakage around blood vessels was stained. Fibrinogen deposition was observed in brains of mice injected with siRNA-chol but not siRNA-EG18 (FIG. 1B). Collectively, these results highlight an improved safety profile for siRNA-EG18 that is more favorable for clinical translation.

### Materials and Method for Examples 9-16

### Synthesis of Oligonucleotides

Oligonucleotide syntheses were performed using standard solid-phase chemistry with a MerMade 12 automated RNA synthesizer (BioAutomation) on controlled pore glass with a universal support (1 or 10 μmol scale, 1000 Å pore), using 2′-F and 2′-OMe phosphoramidites with standard protecting groups (Glen Research). 5′-(E)-Vinylphosphonate (VP) was incorporated on the 5′ terminus of the antisense strand using POM-vinyl phosphonate 2′-OMe-uridine CE-phosphoroamidite (LGC genomics). All strands were grown on the universal support except for Cy5-labelled oligos, which were synthesized on Cy5-functionalized 1000 Å CPG (Glen Research). Amidites were all dissolved at 0.1 M in anhydrous acetonitrile, except for 2′-OMe uridine which requires 20% dimethylformamide as a co-solvent. In addition, the stearyl amidite is dissolved in 3:1 (v: v) anhydrous dichloromethane: acetonitrile (DCM: ACN). A solution of 5-Ethylthio-1H-Tetrazole (0.25 M in acetonitrile) was used as an activator. Detritylation was performed with 3% Dichloroacetic acid in DCM. Capping was done with CAP A (80% Tetrahydrofuran/10% 2,6-Lutidine/10% Acetic Anhydride) and CAP B (20% acetic anhydride, 30% 2,6-lutidine in ACN). Oxidation was carried out with 0.02 M iodine in Tetrahydrofuran/Pyridine/Water (70:20:10) while 0.05 M Sulfurizing Reagent II in Pyridine/Acetonitrile was used as a sulfurizing agent with a reaction time of 5 minutes.

### Cleavage and Deprotection of Oligonucleotides

Unconjugated and conjugated sense strands were cleaved and deprotected with a 1:1 solution of 28%-30% ammonium hydroxide and 40% aqueous methylamine (AMA) for 2 hours at room temperature. Cy5-labelled oligonucleotides were cleaved in 28-30% ammonium hydroxide at room temperature for 20 hours. The VP-containing antisense strands were cleaved and deprotected by treating the CPG with a 3% diethylamine (DEA) solution in 28-30% ammonium hydroxide (20 hours, 35° C.).

### Purification and Characterization of Oligonucleotides

After cleavage and deprotection, oligonucleotides were dried under vacuum to remove solvents (Savant SpeedVac SPD 120 Vacuum Concentrator, ThermoFisher). Pellets were resuspended and purified on a Waters 1525 EF HPLC system equipped with a Clarity Oligo-RP column (Phenomenex) under a linear gradient [60% mobile phase A (50 mM triethylammonium acetate in water) to 90% mobile phase B (methanol)]. Cy5-labeled and unconjugated (DMT-on) sense strands were first desalted using Gel-Pak column (Glen Research) followed by chromatography under a linear gradient (85% to 40% mobile phase A). Oligonucleotide fractions were dried, resuspended in nuclease free water, sterile filtered, and lyophilized. DMT protecting group was removed from the purified, dried, unconjugated strand using 20% acetic acid for 1 hour at room temperature, followed by desalting.

Antisense strands were purified over a 10×150 mm Source 15Q anion-exchange column (Cytiva) using a gradient of sodium perchlorate. Buffer A consisted of 10 mM sodium acetate in 20% acetonitrile and Buffer B consisted of 1 M sodium perchlorate in 20% acetonitrile. The run conditions were 90% to 70% buffer A over 30 minutes at a flow rate of 5 ml/min. Purified oligonucleotides were desalted, sterile filtered, and lyophilized.

The identity of oligonucleotides was verified by Liquid Chromatography-Mass Spectrometry (LC-MS, ThermoFisher LTQ Orbitrap XL Linear Ion Trap Mass Spectrometer). LC-MS was performed using a Waters XBridge Oligonucleotide BEH C18 Column under a linear gradient [85% phase A (16.3 mM triethylamine—400 mM hexafluoroisopropanol) to 90% phase B (methanol)] run for 10 minutes at 45° C.

### In Vitro Assessment of Carrier-Free Oligonucleotide Uptake

Uptake propensity of siRNA conjugates was assessed by treating N2a neuroblastoma cells with Cy5 labeled compounds and measuring fluorescence by flow cytometry. In brief, N2a cells (<P21) were seeded at 100,000 cells/ml onto uncoated 24-well plates and allowed to adhere overnight. The cells were then treated with siRNA-Chol, L2-siRNA, or free siRNA in serum-free Opti-MEM at 60 nM (Cy5 concentration) for 2 hours. Unbound compound was removed with two DPBS−/− washes and then the cells were dissociated using Accutase (Sigma). The collected cells were pelleted and resuspended in flow cytometry buffer (0.5% BSA in DPBS−/−) to run on a Guava EasyCyte (Luminex) flow cytometer. The analysis was performed in FlowJo™ v10.8 Software (BD Life Sciences) to gate single cells (from debris and doublets) and measure geometric mean fluorescence intensity from over 2,000 cellular events.

### In Vitro Assessment of Carrier-Free Gene Silencing

In vitro gene silencing of oligonucleotide conjugates was assessed by carrier-free reverse transfection. Wells were prepared with 250 nM of siRNA in Opti-MEM, then 75,000 N2a cells were added to each well (24-well plate). After 24 hours, an equal volume (500 μl) of full serum DMEM (10% FBS) was added to each well and after an additional 24 hours, cells were harvested for RT-qPCR.

### Animal Husbandry

Adult C57BL/6J male mice were ordered from Jackson Laboratory and used between 10-16 weeks of age. Mice were housed continuously in an environmentally controlled facility in a 12-hour light/dark cycle with ad libitum access to food and water. All mouse protocols were approved by the Institutional Animal Care and Use Committee (IACUC) at Vanderbilt University.

Intracerebroventricular (ICV) injections and euthanasia siRNA duplexes were annealed in 0.9% sterile saline by heating to 95° C. and gradually cooling to 4° C. on a thermocycler. On the day of injection, the compounds were concentrated to either 1.5 mM (15 nmol; for gene silencing) or 1 mM (10 nmol; Cy5-tagged siRNA; for biodistribution) using a 3K Amicon Ultra spin filter (UFC500324). The concentrations were measured by absorbance (260 nm) on a NanoQuant Tecan plate reader and adjusted as necessary by adding saline.

Mice were anesthetized with isoflurane and mounted on the stereotactic rig where they receive continuous isoflurane for the duration of the surgery. Eye ointment (Bausch+Lomb) was administered to prevent drying and then the scalp was sanitized with betadine and 70% ethanol, alternating three times each. The scalp was opened with a midline incision and hydrogen peroxide was applied to expose bregma. Injection coordinates, as distance from bregma, were ±1 medial-lateral, −2.3 dorsal-ventral, and −0.2 anterior-posterior. Two holes were drilled through the skull at these coordinates for bilateral injection. The syringe (Hamilton Model 701, blunt 30 G) was brought to these coordinates and slowly lowered into the ventricle. Injections were performed at 1 μl/min for a total of 5 μl per ventricle. To minimize backflow, the needle was left in the ventricle for an additional 5 minutes prior to gradual retraction. The scalp was then sutured shut, and mice were monitored for their recovery. To maintain body temperature, mice were placed on a heating pad (37° C.) during and after surgery. For analgesia, mice received an intraperitoneal injection of Ketoprofen (5-10 mg/kg Ketofen, Zoetis) prior to surgery and daily for 72 hours post-operation.

At the terminal timepoint, mice were euthanized by ketamine (450 mg/kg)/xylazine (50 mg/kg) overdose and transcardially perfused with cold heparinized (10 U/ml) DPBS−/− to remove blood cells from the vasculature. For flow cytometry studies, the right hemisphere was extracted into DPBS and processed into single cells as described below. For gene silencing studies, brains were extracted after perfusion and cut into 1 mm slices using a sagittal brain matrix. Biopsy punches (2 mm) were taken from different brain regions (hippocampus, striatum, cerebellum, posterior cortex, brainstem) and stored in RNAlater (Thermo AM7020) for downstream analyses. Spinal cords were extracted by extrusion with HBSS and segmented into cervical, thoracic, and lumbar regions using the characteristic enlargements as a guide. For biodistribution studies, mice were further perfused with 4% paraformaldehyde (PFA), and then brains and spinal cords were extracted, immersion fixed in 4% PFA overnight at 4° C., and subjected to further downstream histological processing. Organs were also harvested and Cy5 fluorescence was measured by IVIS Lumina III imaging (Caliper Life Science, Hopkinton, MA).

### Peptide Nucleic Acid (PNA) Hybridization Assay

A PNA assay was used to quantify absolute siRNA delivery after ICV administration in mice. Conceptually, the siRNA duplex is denatured into single strands, and a Cy3-PNA probe with complete complementarity to the antisense strand forms a new PNA-anti-sense duplex. When run through an anion-exchange column, the PNA-RNA duplexes elute in a distinct peak, and the area under the curve can be related to mass of siRNA using a standard curve where known amounts of siRNA are doped into tissue homogenates from untreated mice or rats. Data are reported as mass of antisense strand per mass of tissue.

To prepare homogenates, tissue biopsy punches were removed from RNAlater, placed in 300 μl homogenization buffer (Thermo QS0518) plus proteinase-K (Thermo QS0511, 1:100), and disrupted using a Tissuelyzer 2.0 for 5 minutes at 30 Hz. Following a 1-hour incubation at 65° C., the samples were spun down at 15,000 G for 15 minutes and the supernatant was collected for storage at −80° C. The standard curve was prepared at the same time as the homogenates, with a maximum of 10,000 fmol and minimum of 156.25 fmol by 1:2 serial dilution. A new standard curve was prepared for each conjugate type and siRNA sequence.

Samples were thawed and sodium dodecyl sulfate (component of homogenization buffer) was precipitated from 200 μl of homogenate with 20 μl of 3M potassium chloride and centrifuged at 4,000×g for 15 min. The supernatant was collected and centrifuged at the same speed for an additional 5 minutes to ensure complete removal of the precipitate. If samples required a dilution to fall within the standard curve, they were diluted to 200 μl in homogenization buffer prior to precipitation. Next, 150 μl of supernatant was transferred to a screw cap tube, where 100 μl of hybridization buffer (50 mM Tris, 10% ACN, PH 8.8) and 2 μl of 5 M PNA probe (˜10 μmol/150 μl of sample, PNA bio) were added. The probe was annealed to the antisense strand by heating to 90° C. and then 50° C. for 15 minutes each. The samples were then run through a DNAPac PA100 anion-exchange column (Thermo Fisher Scientific) on an iSeries LC equipped with RF-20A fluorescence detector (Shimadzu). Mobile phases consisted of buffer A (50% acetonitrile and 50% 25 mM Tris-HCl, pH 8.5; 1 mM ethylenediaminetetraacetate in water) and buffer B (800 mM sodium perchlorate in buffer A), and a gradient was obtained as follows: 10% buffer B within 4 minutes, 50% buffer B for 1 minutes and 50% to 100% buffer B within 5 minutes. 58 The final mass of siRNA was calculated using the area under the curve of Cy3 fluorescence from a standard curve of known quantities of siRNA or L2-siRNA spiked into untreated tissue homogenates.

### RT-qPCR

RT-qPCR methodology was employed to determine mRNA silencing in vitro and in vivo. For in vitro studies, cells were lysed in RLT buffer plus β-mercaptoethanol (1:100) and RNA was extracted using an RNeasy plus mini kit (Qiagen 74134). Reverse transcription into cDNA was performed according to iScript manufacturer instructions (iScript cDNA synthesis kit, Biorad 1708891). Gene expression was measured by Taqman qPCR on the cDNA using 20 μl reactions, run on a Biorad CFX96, and analyzed in CFX Maestro software.

For in vivo studies, brain homogenates were prepared in 350 μl of RLT buffer plus β-mercaptoethanol and processed with 5 mm stainless steel beads (Qiagen cat. No. 69989) for 5 minutes at 30 Hz (TissueLyser II). RNA was then extracted using an RNeasy plus micro kit (Qiagen 74034) according to manufacturer instructions. RNA was eluted in 14 μl RNAse-free water and the concentration and purity were measured by 230/260/280 nm absorbance on a Nanodrop 2000c spectrophotometer. Last, qPCR was performed by preparing a 10 μl reaction mixture composed of 2× master mix, water, Taqman probes, and cDNA sample. Taqman probes were Mm00478295_m1 (Ppib), Mm01213820_m1 (Htt), and Mm02619580_g1 (Actb). In vivo samples were run on a Quant 12k flex in triplicate (2 min @ 5, 10 @ 95° C., then cycle 15 seconds @ 95° C. and 1 min @ 60° C.). All samples were analyzed according to standard ΔΔCt methodology. Each sample is normalized to Ppib as a housekeeping gene unless Ppib is the target of the siRNA, in which case Actb is the housekeeping gene. Conventional RT-qPCR controls (no-template control and no reverse transcriptase control) were run on every plate and did not amplify.

### Western Blotting

Brain homogenates were prepared in freshly made lysis buffer containing 10 mM HEPES (Sigma, adjusted to pH 7.2), 250 mM sucrose (Sigma), 1 mM EDTA, 1 mM sodium fluoride, 1 mM sodium orthovanadate, and 1 protease inhibitor tablet (Roche 11836170001). 75 μl of buffer was added to each biopsy punch, and samples were homogenized three times for 10 seconds with a handheld homogenizer. Samples were cooled on ice for 30 seconds between homogenizations to prevent protein degradation. All homogenates were then sonicated (handheld) for 20 seconds to liberate nuclear proteins, and samples stored at −80° C. until further analysis. Bicinchoninic acid (BCA) assay (Thermo 23225) was used to quantify protein and normalize loading of each sample. In brief, 10 μl of sample was added to each well in duplicate followed by the addition of 200 μl of substrate. The plate was incubated for 30 minutes at 37° C. in the dark. Absorbance was measured at 562 nm and protein was quantified from a bovine serum albumin standard curve.

To prepare samples for Western blotting, 10 μg of each sample was diluted with TBS in XT sample buffer (Biorad 1610791) plus 5 mM dithiothreitol (dtt) and beta-mercaptoethanol (1:10) and heated for 5 minutes at 95° C. to fully denature proteins. Samples were loaded on a 3-8% tris-acetate gel (Biorad 3450131) in Tricine running buffer (Biorad 1610790) or Novex tricine SDS running buffer (LC1675). The gel was run at 120-150V until the loading dye ran off the bottom. Next, the gel was transferred to a nitrocellulose Midi membrane (Biorad 1704159) using a Trans-Blot Turbo (Biorad). This membrane was then blocked for 1 hour in 5% non-fat milk (Bob's red mill) diluted in TBS. Primary antibodies were added overnight in 5% milk in TBS+0.1% tween-20 (TBST): huntingtin (Clone D7F7, Cell Signaling 5656, 1:1,000), beta-actin (Clone 13E5, Cell signaling 4970, 1:2,000), GFAP (Clone D1F4Q, Cell signaling 12389, 1:2,000). After rocking at 4° C. overnight, the membrane was washed four times for 10 minutes with TBST. The HRP-conjugate secondary antibody (Abcam ab6721) was diluted 1:20,000 in TBST and applied at room temperature for 1 hour. When applicable, GAPDH (HRP-60004 Proteintech, 1:3,000) was added with the secondary. After washing thrice with TBST and once with TBS for 10 minutes, the HRP substrate was added to visualize the bands. For huntingtin detection, super signal west femto substrate (Thermo 34095) was used, while Clarity Western ESC (Biorad 1705060) was used for beta actin detection. Blots were imaged for chemiluminescence on a Bio-rad ChemiDoc MP imaging system and analyzed in ImageLab to measure total band intensity.

### Histology

To prepare fixed brains for frozen sections, sucrose gradients were performed for cryoprotection by immersion in 15% sucrose for 24 hours (or until the sample sinks), followed by immersion in 30% sucrose for an additional day. The hemispheres were then embedded in Epredia Neg-50 medium, sectioned to 30 μm on a cryostat, and stored at −80° C. until staining.

Antibody staining was performed as follows. First, samples were thawed to room temperature and a barrier was drawn using a hydrophobic pen to localize the staining reagents on the sample. Samples were washed in DPBS−/− with 0.3% Triton-X 100 (PBST), followed by blocking for 1 hour at room temperature in PBST plus 5% donkey serum (Sigma D9663). Slides were then incubated either 2 hours at room temperature or overnight at 4° C. with the primary antibody diluted in DPBS containing 1% BSA and 0.5% Triton-X 100. Primary antibodies include CD31 (1:100, BD biosciences 550539), Lyve1 (1:200, R&D AF2125), AQP4-488 (1:500 for overnight or 1:200 for 2 hours, Abcam ab284135), Glut1-PE (1:300, Abcam ab209449), AQP1 (1:200, Abcam ab168387). The sections were then washed three times in PBST for 5 minutes, followed by a 1-hour room temperature incubation in the appropriate secondary antibody (1:500 dilution in DPBS containing 1% BSA and 0.5% Triton-X 100). Sections were washed, then incubated with DAPI (1:5,000-1:10,000, 5 mg/ml stock, Thermo D1306) for 10 min, washed again in DPBS and then mounted under a coverslip with ProLong gold antifade reagent. Imaging was performed either on a Leica epifluorescence microscope (tiled images of entire brain) or a Zeiss LSM 710 confocal microscope (higher magnification of select regions). Image processing was performed using Fiji software. For samples where antibody staining was not required (i.e. assessing Cy5-tagged siRNA signal), slides were washed twice with DPBS and then mounted with ProLong gold plus DAPI.

Toxicity immunohistochemistry was performed on paraffin embedded sections using the Epredia Autostainer 360. Sections were baked at 60° C. for 1 hour then deparaffinized and rehydrated through xylene and ethanol steps. Antigen retrieval was performed using the Epredia PT module in citrate buffer (pH 6.0) at 97° C. Slides were then transferred to the autostainer and blocked with Dako Protein Block (X0909) and Flourescent block (ThermoFisher 37565). Primary antibodies were diluted in Dako protein block and incubated on the slides for 1 hour at room temperature. Astrocytes were labeled with anti-GFAP (1:1000, Dako Z0334) and microglia with anti-Iba1 (1:500, Wako 019-19741). Slides were then washed and incubated in Alexaflour 647-conjugated secondary antibody (1:1000, ThermoFisher A21245) for 1 hour at room temperature. Sections were mounted with prolong gold with DAPI (Invitrogen P36931) and imaged using the Aperio Versa 200 slide scanner. All quantification was done using ImageJ.

### Tissue Clearing

Mouse brains or spinal cords were incubated for 3-5 days in 4% PFA and the right hemispheres were embedded in CLARITY polyacrylamide hydrogel (4% Acrylamide, 0.05% Bis-Acrylamide, 0.25% temperature-triggering initiator VA-044 in 0.1 M PBS). To allow time for the polyacrylamide to permeate, the tissue remained in unpolymerized solution at 4° C. for 2 weeks. Tubes were then moved to a 37° C. water bath for 4 hours to activate the VA-044 acrylamide crosslinker and polymerize the hydrogel. After the polymerization, tissue was cleared passively with a clearing solution (200 mM boric acid, 4% w/v SDS, pH 8.5) at 37° C. shaking in a humidified incubator for 4-8 weeks. To stain for vasculature, the sample was washed overnight in PBS (0.1 M) with 0.1% Triton-X and then incubated in the same buffer containing lectin-fluorescein (0.5 mg/ml, FL-1171, Vector Laboratories), overnight for spinal cords and two days for brains. The same wash was repeated overnight, and afterwards the samples were incubated in 68% Thiodiethanol (TDE) overnight for refractive index matching (1.33). The samples were imaged on a light-sheet Z1 microscope (Zeiss) using 20× objective illumination and processed with Imaris software (version 9.9.0, Bitplane, USA).

### Fast Protein Liquid Chromatography (FPLC)

FPLC was used to assess binding of different compounds to albumin in human CSF. Cy5-labeled conjugates or free siRNA (1 μM) were mixed with 300 μl post-mortem human CSF or human serum albumin (7.5 UM). After 30 minutes of incubation at 37° C., the volume was then brought to 1 ml in running buffer (10 mM Tris-HCl, 0.15 M NaCl, 0.2% NaN3), filtered (0.22 μm, Millipore UFC30GV00), and fractionated by size-exclusion through three tandem superdex 200 increase columns on an Akta Pure FPLC (GE Healthcare). The siRNA content of each fraction was measured with a plate reader (Biotek Synergy H1) for Cy5 fluorescence. A Western blot for albumin was performed to determine elution peaks of albumin in CSF.

### Flow Cytometry

Harvested brain tissue was converted to single cell suspensions using the papain-based mouse adult brain dissociation kit from Miltenyi Biotec (130-107-677), with appropriate steps for myelin removal and red blood cell lysis according to the manufacturer's instructions. Cells were then FcR-blocked for 10 minutes on ice (10 μl per sample, Miltenyi 130-092-575). The cells from each brain were then split into 4 experimental samples-one for each cell-specific antibody cocktail: Thy1 for neurons (1:5,000; Biotechne FAB7335P), ACSA2 for astrocytes (1:2,000; Miltenyi 130-123-284), CD11b for immune cells (1:2,000; BD biosciences 561689), and CD31 for endothelial cells (1:2,000; eBioscience 17-0311-80). Each population was additionally stained with 01 (1:100, R&D systems FAB1327G), a marker for oligodendrocytes, which are excluded from analysis. After washing, cells were resuspended in DPBS−/− with 0.5% BSA and DAPI (1:10,000, 5 mg/ml stock) and run on an Amnis CellStream flow cytometer. 25,000 cellular events were recorded. The gating scheme to identify Cy5+ cells leveraged fluorescence-minus-one (FMO) controls, which are samples stained with identical antibodies to the experimental groups but lack Cy5 signal (originating from an uninjected mouse). Single color compensation controls were run for each fluorophore. To validate ACSA2 gating of astrocytes, cells were sorted on a 4-laser BD FACSAria Ill and RT-qPCR was performed as described using taqman probes to show enrichment of astrocytes (Mm01253033_m1) and depletion of endothelial cells (Mm00727012_s1).

### Multiplexed Analysis of Cytokines

Mice were administered 15 or 5 nmol of L2-siRNA by ICV injection as previously described. Two weeks after ICV injection, the cortex and striatum were biopsy punched, flash frozen, and stored at −80° C. To prepare homogenates, samples were thawed in cell lysis buffer (Biorad 171304006M) and homogenized three times for 10 seconds with a handheld homogenizer, with 30 second intervals on ice between homogenizations. The solution was then sonicated for 30 seconds and centrifuged at max speed for 10 minutes at 4° C. Supernatant was collected and total protein was quantified by BCA assay as previously described. The samples were then diluted to 1 mg/ml and processed by Eve Technologies using the Mouse High Sensitive 18-Plex Discovery Assay according to the manufacturer's protocol (Millipore Sigma).

Single-Cell RNA Sequencing (scRNA-Seq) Sample Preparation

Mice were administered 15 nmol of L2-siRNA by ICV injection as previously described. The siRNA sequence was targeted to Ppib or a non-targeting control. After 1 month, mice were perfused with heparinized (10 U/ml) DPBS and a single cell suspension of the brain was prepared using the adult brain dissociation kit as previously described. CD11b positive and negative cells were isolated via MACS sorting with Cd11b-coated microbeads (Miltenyi 130-097-142) according to the manufacturer's instructions. After 2 washes with cell-suspension buffer, cells were counted on a hemocytometer and diluted to target 2,000 (Cd11b^{pos}) or 20,000 (Cd11b^{neg}) cells for encapsulation. Unencapsulated cells were used for RT-qPCR as previously described. The single cell libraries were prepared using the PIPseq T2 (for Cd11b^{pos}) or v4.0 PIPseq T20 kits (for Cd11b^{neg}) following manufacturer specifications. Reads were generated at a depth of 40 million reads for CD11b^{pos} and 400 million read for CD11b^{neg} from an Illumina NovaSeq6000 PE150 sequencing run, yielding a final read depth of 20K reads per cell.

scRNA-Seq Data Processing

Reads were processed using the PIPseeker alignment algorithm (v02.01.04). Counts matrices were then processed using standard techniques in Seurat (v4). Datasets were merged without batch correction, and cells were filtered based on number of features, keeping cells with 800-4,000 uniquely expressed genes for samples processed with T2 kits and 800-10,000 for samples processed with T20 kits. Counts were then normalized, and the dataset was reduced to the top 2,000 variable genes. The data were then scaled, and dimension reduction (PCA and UMAP) was performed based on the first 50 principal components to visualize the data. Cells were clustered using the Louvain algorithm and annotated based on standard marker genes according to the mouse brain atlas. 59 Average Ppib expression levels were calculated using the AverageExpression( ) function in Seurat and were reported per cluster per biological replicate. For barplots, expression levels were normalized to L2-siRNA^{NTC} control.

### Example 9

### Lipid-siRNA Conjugate Structure and Properties

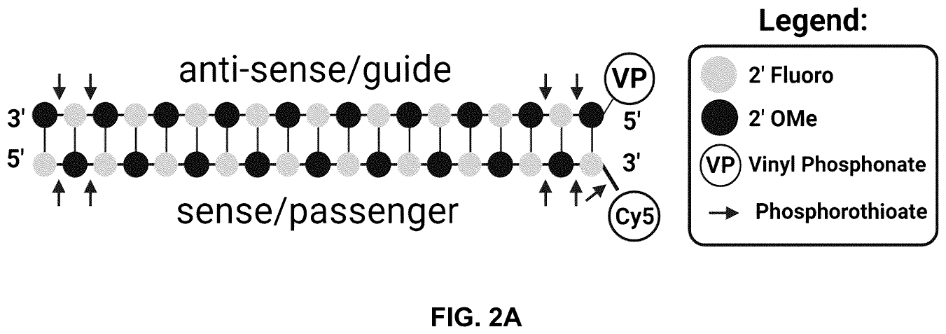

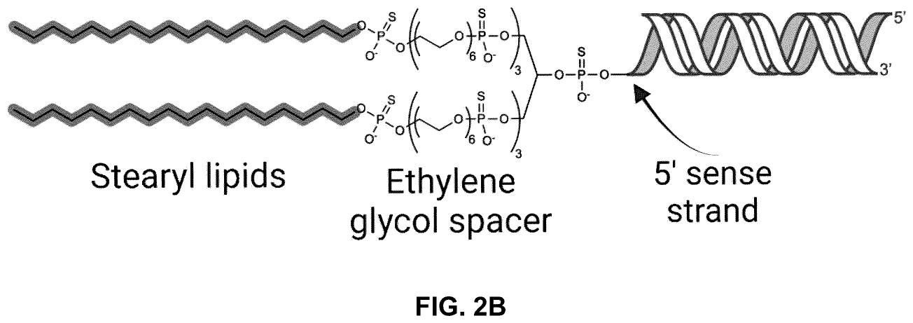

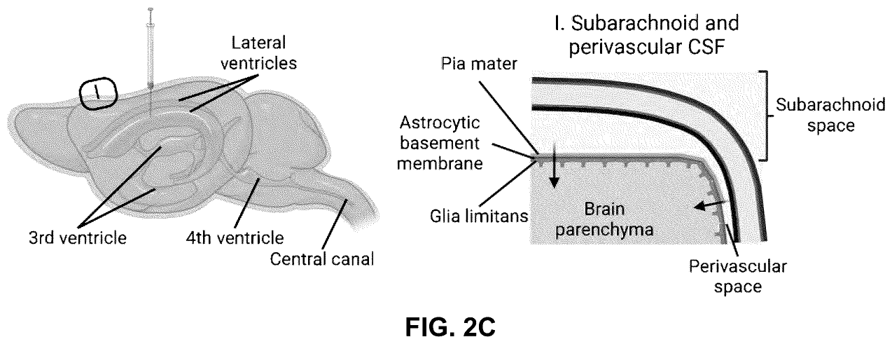

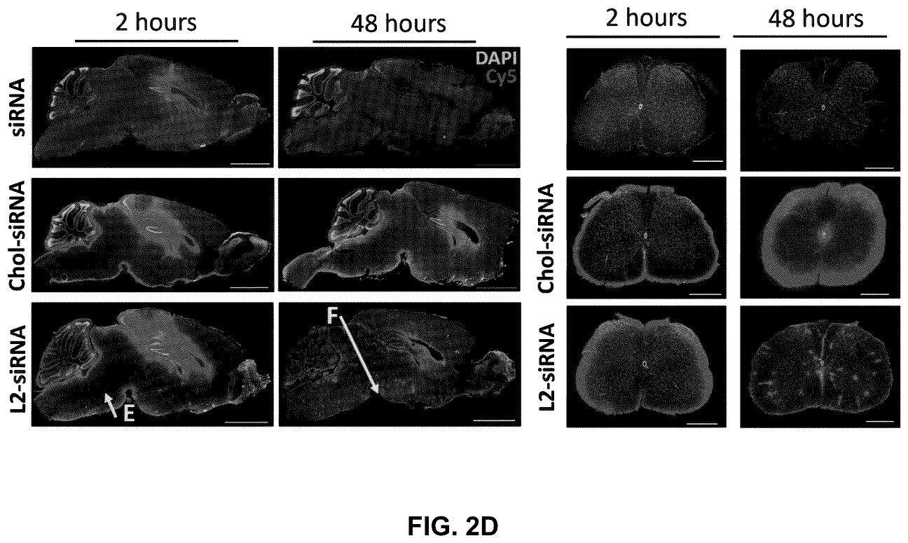

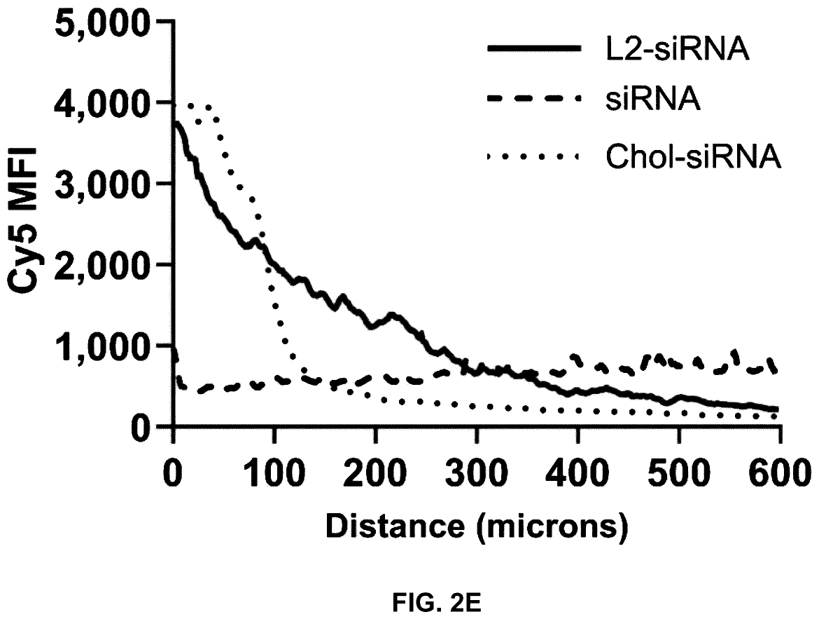

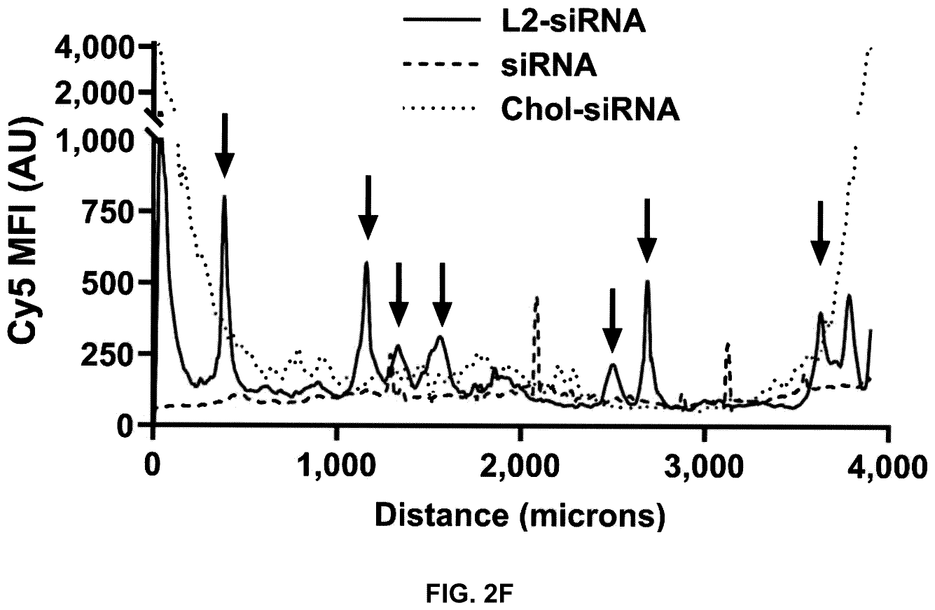

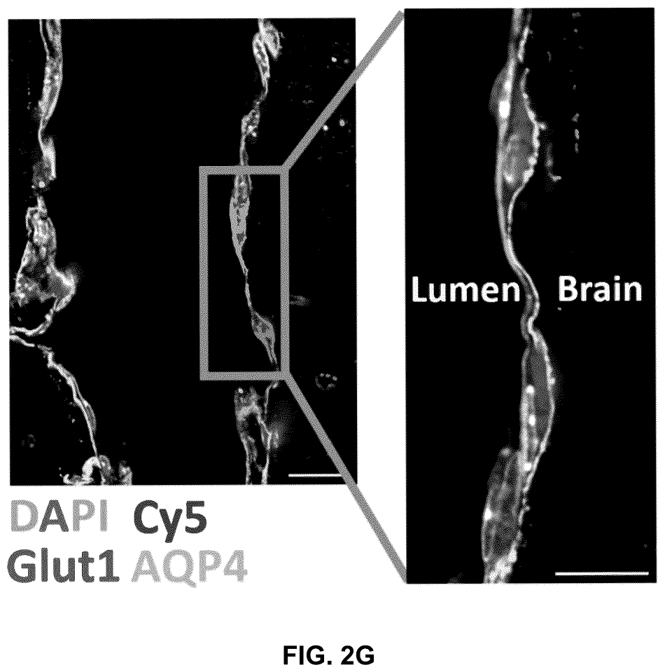

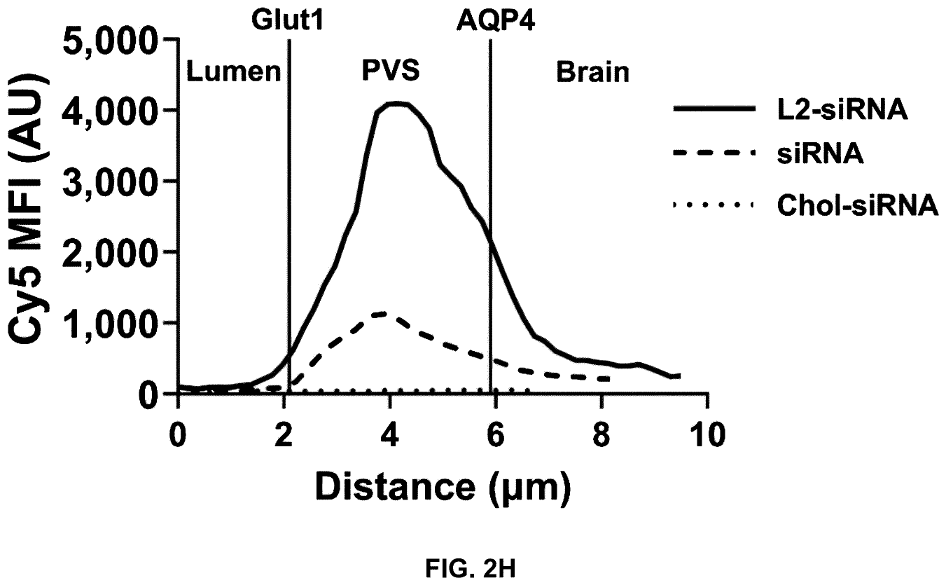

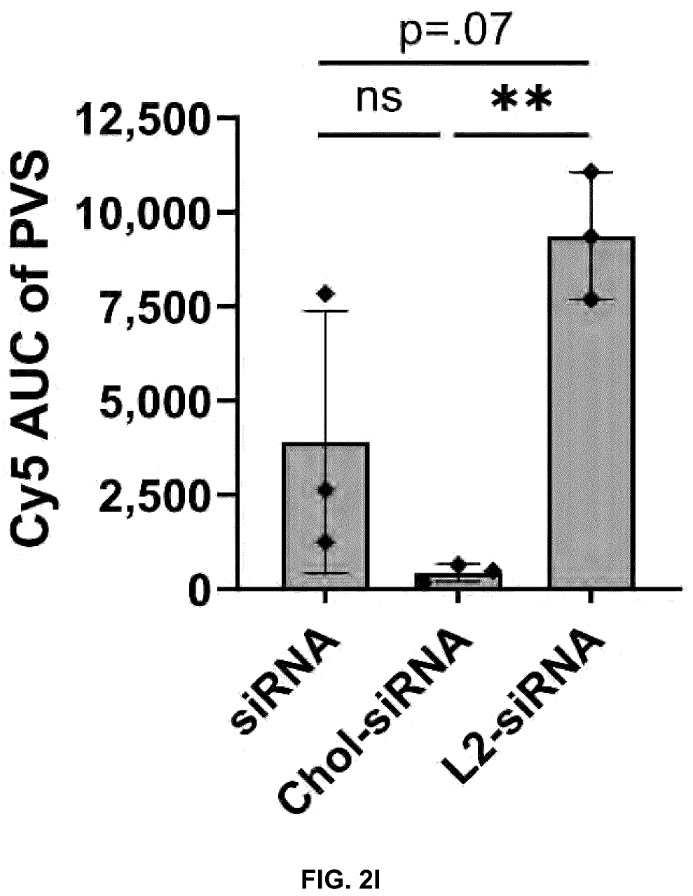

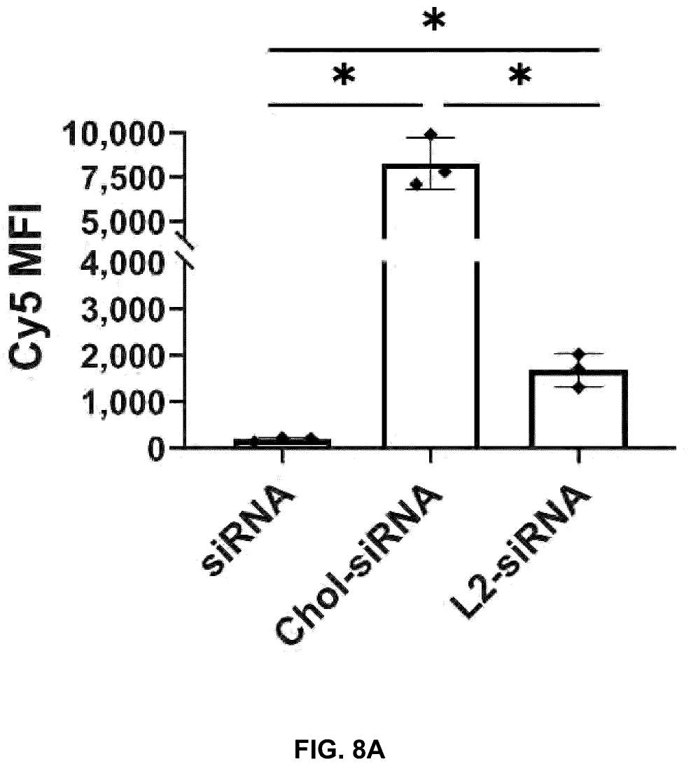

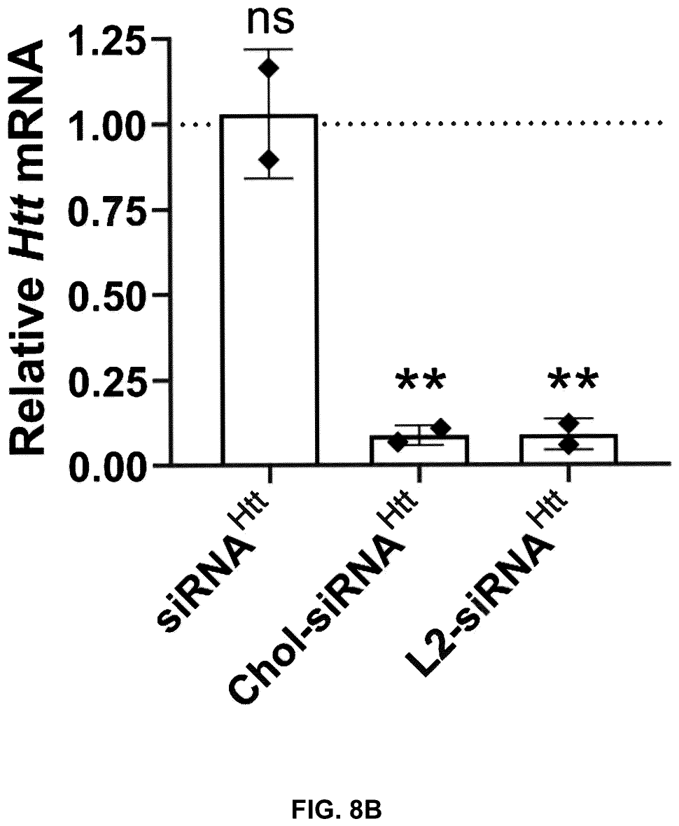

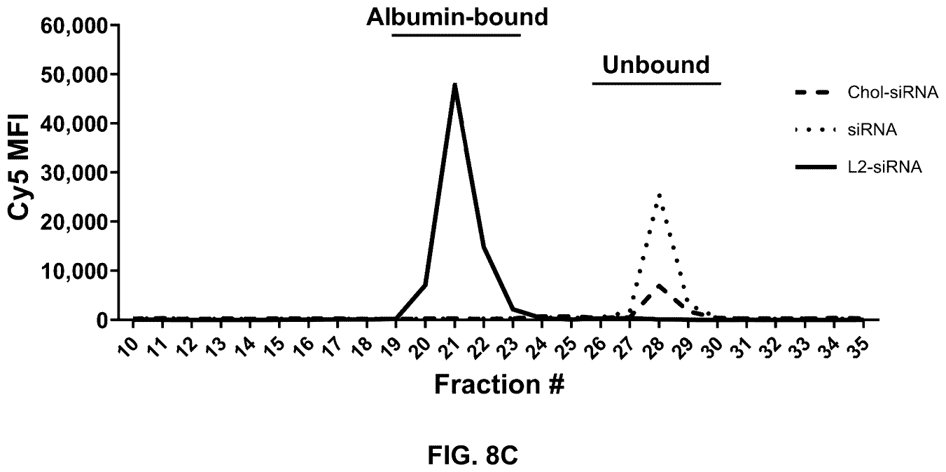

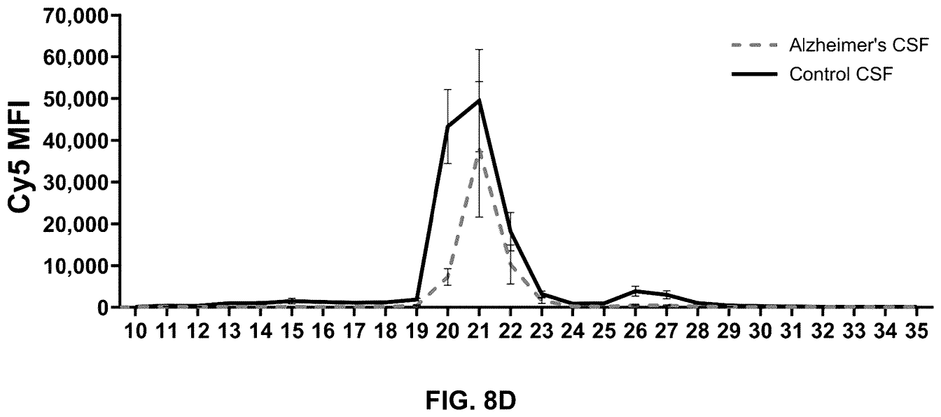

To initially examine how the properties of L2-siRNA might impact CSF to brain delivery, in vitro uptake in neuroblastoma cells and ex vivo association with albumin in CSF were evaluated, with comparisons to unconjugated siRNA (“siRNA”) and cholesterol-conjugated siRNA (“Chol-siRNA”). The blunt-ended siRNA design contains alternating 2′Ome and 2′F on both strands in a “zipper” pattern; also, phosphorothioate (PS) linkages were used between the last two bases on the ends of both strands, and vinyl phosphonate was integrated at the 5′ antisense position, an important feature for maximal silencing in the CNS. Structurally, the L2-siRNA branches off the 5′ end of the sense strand and contains a spacer (18-ethylene glycol repeats) followed by divalent lipid tails (two 18-carbon stearyl groups) (FIG. 2A and FIG. 2B). The L2-siRNA construct showed higher cell uptake than the parent siRNA, while Chol-siRNA, a benchmark chosen for its known cell penetration capacity, had the highest level of uptake in serum-free media (FIG. 8A). Despite uptake differences, L2-siRNA carrier-free gene silencing activity was equivalent to Chol-siRNA at 250 nM (FIG. 8B). In addition, L2-siRNA associated with albumin in ex vivo human CSF, as demonstrated by fast protein liquid chromatography (FPLC); in comparison, Chol-siRNA and unconjugated siRNA showed no association with albumin (FIG. 8C and FIG. 8D). These outcomes suggested that L2-siRNA is a good candidate to potentially leverage albumin for perivascular transport, while also having sufficient capacity for intracellular delivery within the CNS.

### Example 10

L2-siRNA Along Perivascular Spaces and can Enter the Brain by Diffusion from the Subarachnoid Compartment

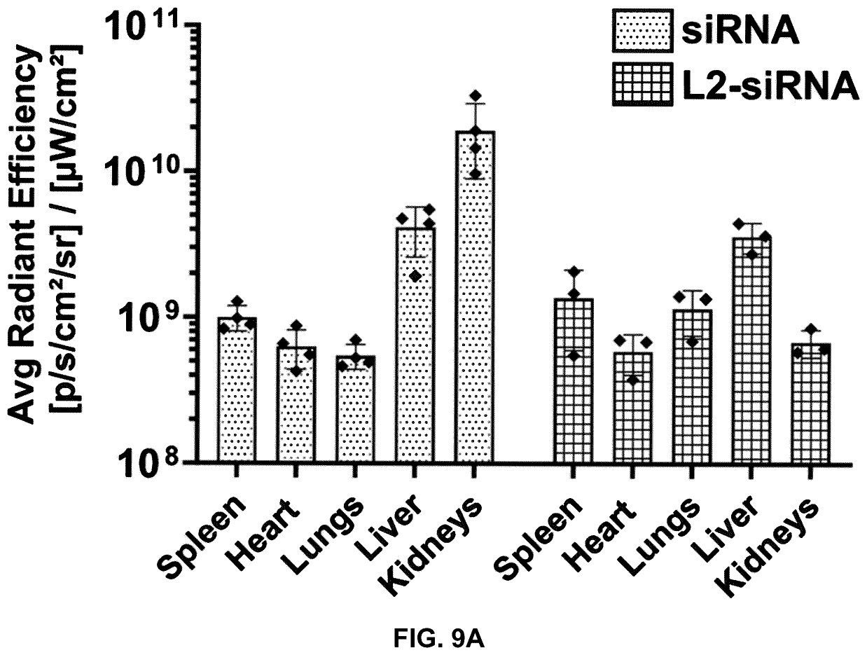

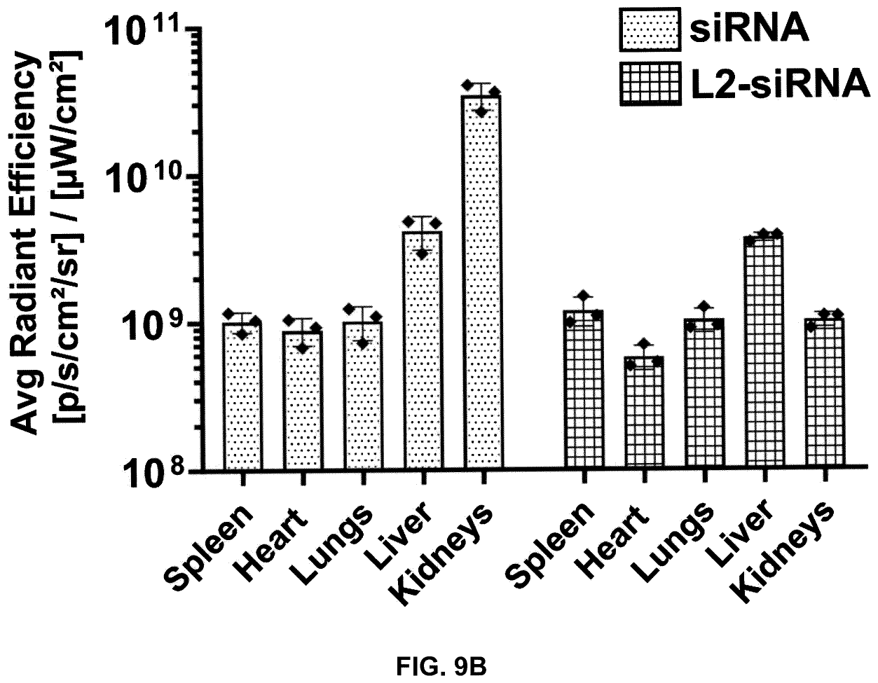

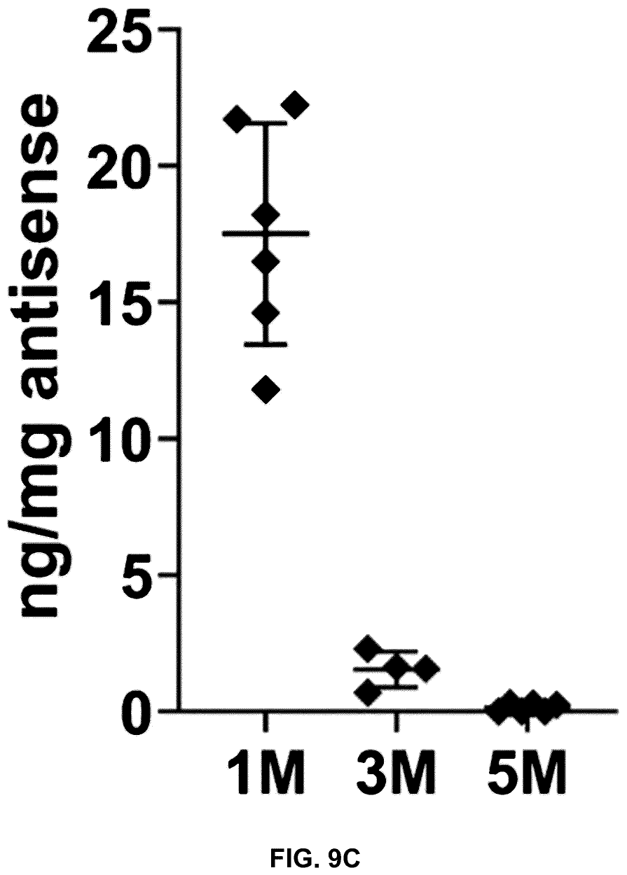

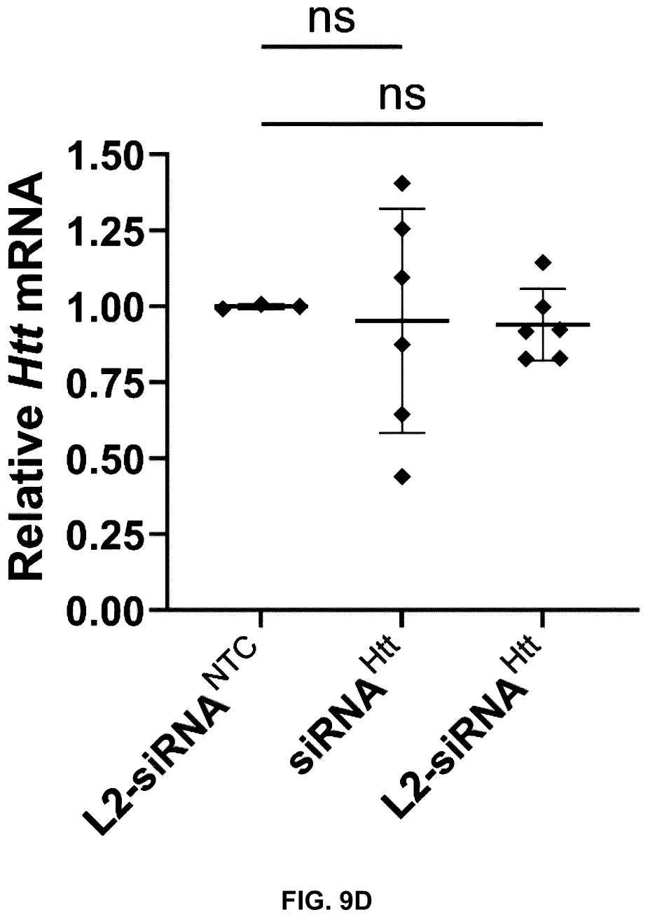

To investigate how lipid properties impact CSF to brain transport in vivo, mice were injected ICV with Cy5-labeled unconjugated siRNA, Chol-siRNA, or L2-siRNA and biodistribution was examined after 2 and 48 hours (FIG. 2D). It was observed that unconjugated siRNA disperses through the mouse brain and spinal cord at 2 hours, but its signal dropped precipitously by the 48-hour timepoint (FIG. 2D). Coupled with the concomitant accumulation of siRNA in kidneys at 2 hours (FIG. 9A and FIG. 9B), these data suggest that siRNA is poorly taken up by CNS cells and swiftly cleared from CSF. In contrast, Chol-siRNA exhibited the most signal at both timepoints, but inhomogeneous dispersion was observed, characterized by steep concentration gradients away from the CSF-brain interface (FIG. 2D). This pattern was consistent in the spinal cord, where Chol-siRNA exhibited steep gradients from the subarachnoid space and central canal (FIG. 2D). In contrast, L2-siRNA transport was evident across all CSF interfaces in the parenchyma (FIG. 2D): ventricular (trans-ependymal), subarachnoid (trans-pial, including cisternal; FIG. 2E), and perivascular. In the spinal cord, L2-siRNA also exhibited broad tissue dispersion along with observable puncta (FIG. 2D), presumably associated with perivascular compartments.

The localization of L2-siRNA to perivascular spaces was next assessed. First, the Cy5 intensity profile was measured across the midbrain to the interpeduncular cistern (far from injection site) and peaks of L2-siRNA were detected consistent with perivascular transport to deep brain structures (FIG. 2F). Localization within the perivascular space was confirmed by the presence of L2-siRNA between endothelial cells (Glut1+) and the astrocytic parenchymal glia limitans (Aquaporin-4+) (FIG. 2G). Measuring Cy5 intensity between these markers confirmed that L2-siRNA achieves greater perivascular delivery than Chol-siRNA (FIG. 2H and FIG. 2I). To evaluate whether delivery to perivascular spaces could be an artifact of the injection volume (e.g. pressure-driven effects), mice were injected ICV with either 2 μl or 10 μl of L2-siRNA at equivalent total dose (2 nmol). It was found that L2-siRNA similarly accessed perivascular spaces in the brain and spinal cord at the lower volume, suggesting the molecular properties of L2-siRNA are responsible for observed outcomes. These results indicate that L2-siRNA delivered into the CSF is trafficked through perivascular spaces, providing a route for transport into deeper parenchymal structures.

CSF to brain delivery is driven by a combination of diffusion from subarachnoid/ventricular CSF and convective movement along perivascular spaces. The Examples suggest that unconjugated siRNA swiftly disperses throughout the CNS via both pathways, but is not effectively internalized by or retained within cells, as evidenced by minimal detection 48 hours after injection and rapid clearance to peripheral organs. While cholesterol conjugation has been used to promote cell uptake, the Examples generally follow prior studies showing that Chol-siRNA cell-association prevents effective transport across large distances. In contrast, L2-siRNA achieves favorable balance of cell internalization and CSF convective transport, distributing further and more homogeneously into the parenchyma from subarachnoid CSF by accessing the extensive perivascular CSF flow network. It is thought that this transport pathway may be even more important for achieving deep, brain-wide delivery in larger organisms following administration into CSF.

The precise mechanisms governing drug transport through the perivascular spaces are not fully understood, but they are thought to be somewhat size dependent. For antibodies, single domain constructs exhibit greater perivascular delivery relative to full length antibodies. This suggests that relatively small nucleic acid therapies are well-suited for this transport, and indeed siRNA (small, negatively charged linear molecules) was observed readily accessing the entire perivascular network 2 hours after ICV delivery. Yet, the conjugation of cholesterol severely limits penetration into these spaces, with minimal perivascular signal beyond superficial vessels likely due to extensive cellular uptake. It was recently shown that ASOs can be transported along the perivascular route, similar to the parent siRNA and L2-siRNA conjugate, but that depth of delivery was dose dependent, with deep brain structure perivascular signal only evident at the highest doses administered intrathecally in rats. Depending on their chemical composition (particularly the number of phosphorothioate bonds), ASOs can weakly bind many proteins in plasma, including albumin. Along these lines, L2-siRNA association with albumin could be one of the driving forces underlying enhanced perivascular transport. It is hypothesized, without being bound by a particular theory, that albumin occupancy of the L2 lipids temporarily reduces its interaction with cell membranes to enable greater dispersion throughout CSF compartments. Importantly, this interaction is reversible and when L2-siRNA transiently dissociates from albumin, the freed lipids can facilitate efficient cell membrane binding and penetration.

### Example 11

### L2-siRNA Achieves Durable Gene and Protein Silencing in Bulk Tissue Across Multiple Brain Regions

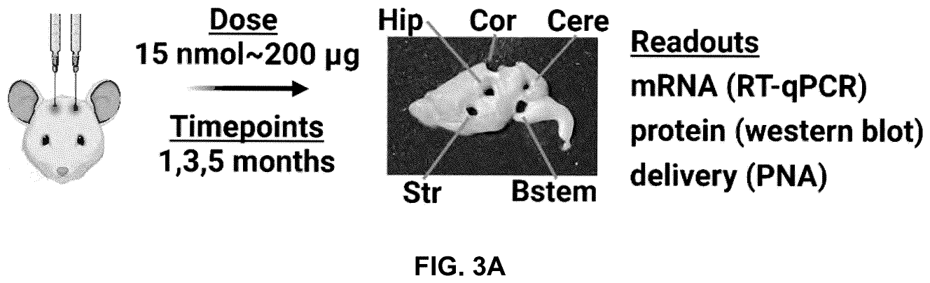

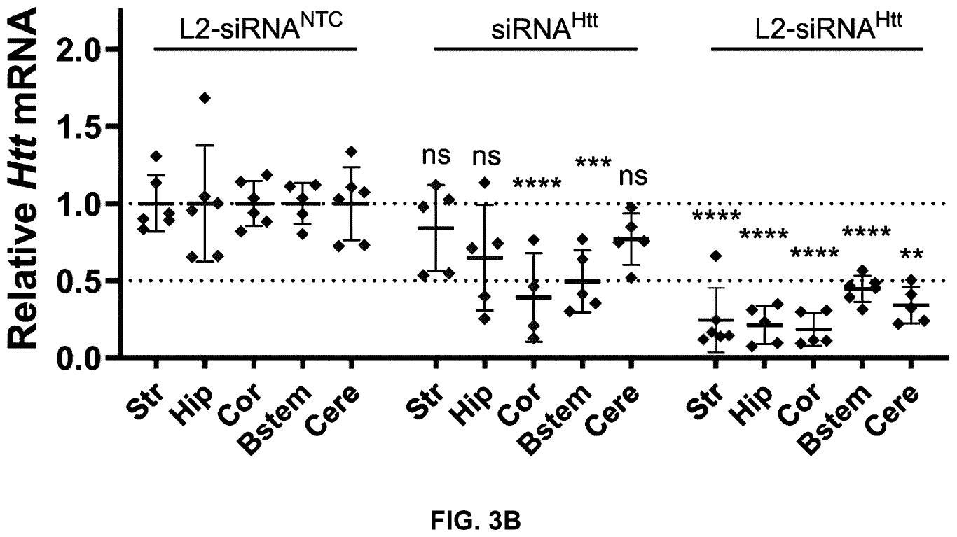

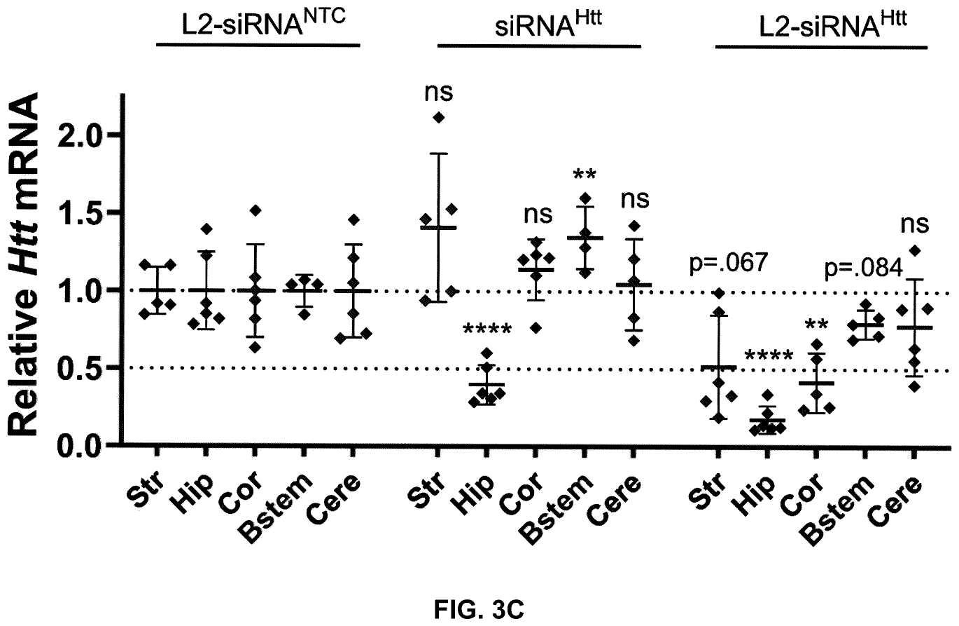

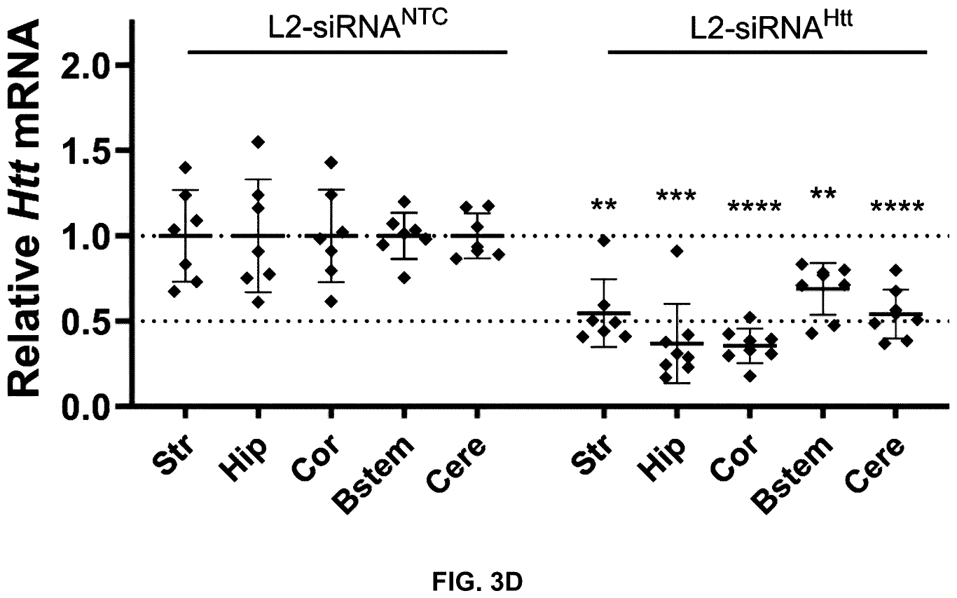

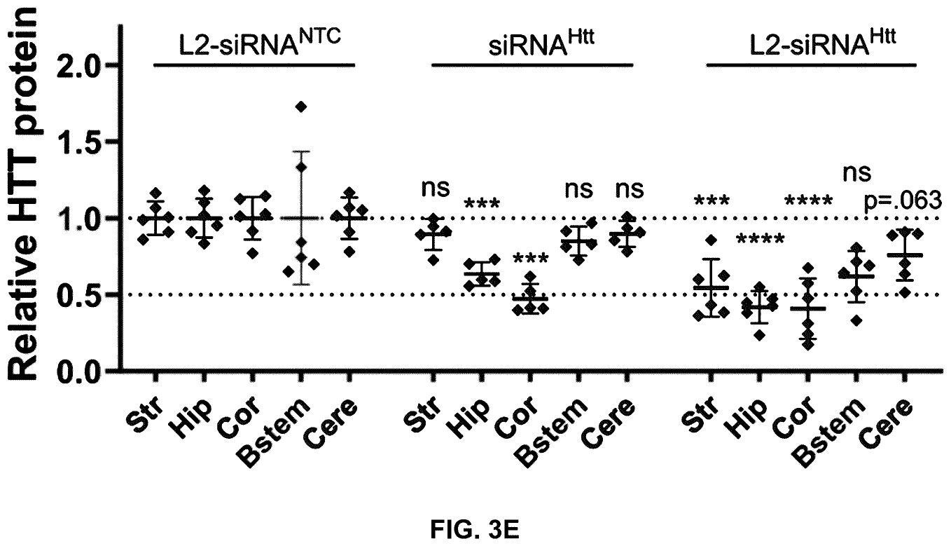

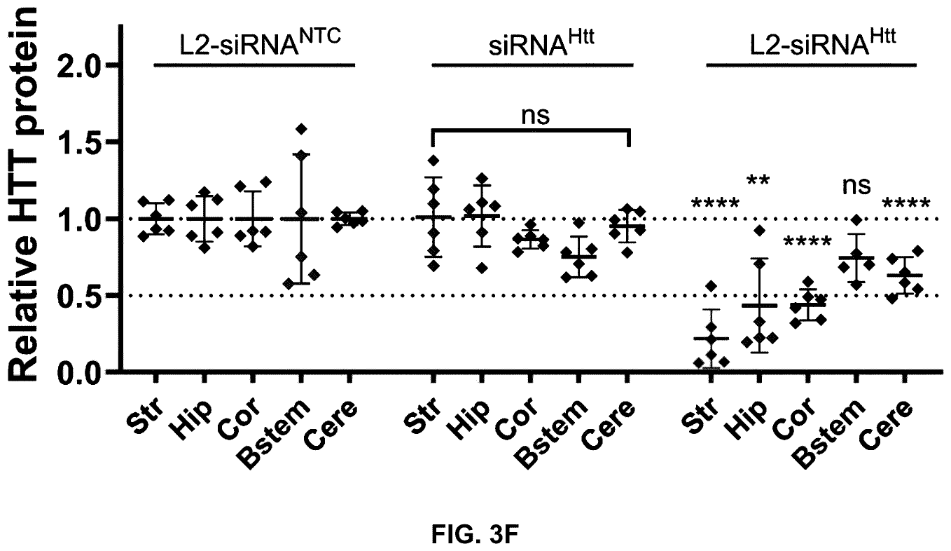

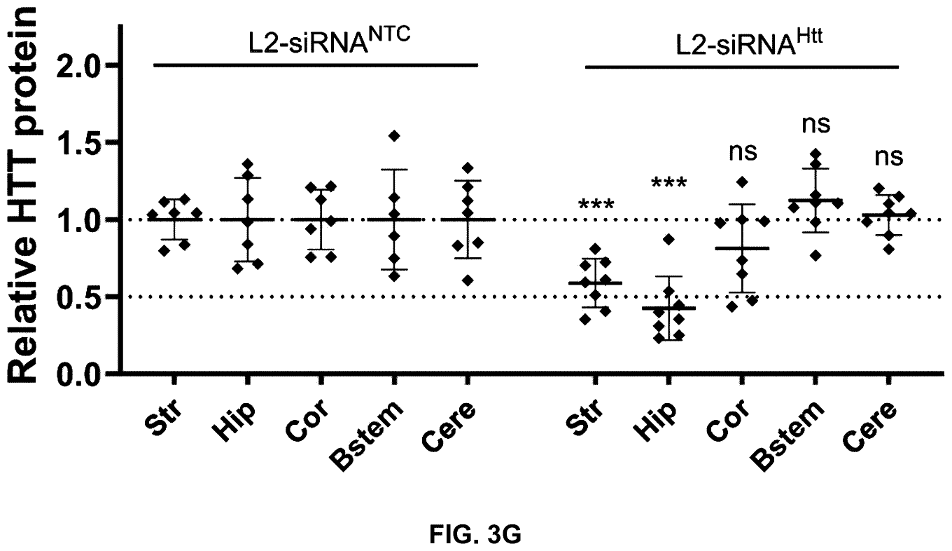

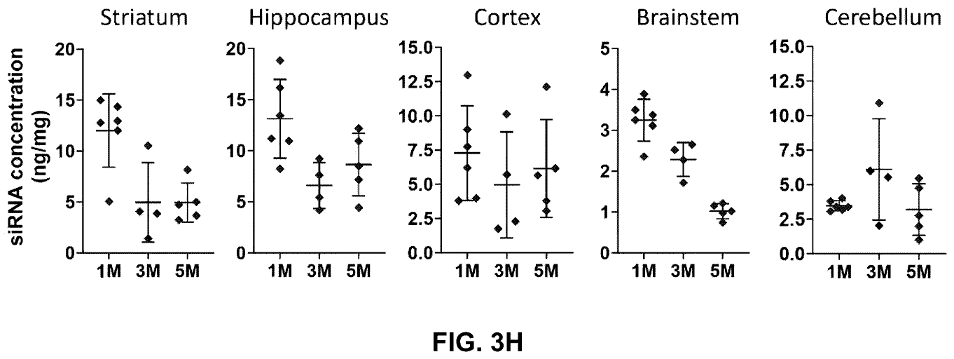

Based on the promising delivery profile of L2-siRNA, the potency and kinetics of target gene silencing was characterized. Htt was chosen as a disease-relevant target that is ubiquitously expressed by CNS cells and for which a well-validated siRNA sequence exists (“Htt10150”). Adult mice were administered 15 nmol ICV, a relatively low dose compared to other nucleic acid therapeutics targeting Htt in mice, such as a divalent siRNA structure (40 nmol) or clinical ASO (˜100 nmol administered over two weeks). Htt mRNA was measured by RT-qPCR, HTT protein by Western blotting, and absolute amount of anti-sense strand delivery with a peptide nucleic acid (PNA) hybridization assay; analyses were performed for CNS regions both proximal to the lateral ventricle injection site (striatum, hippocampus, cortex) as well as distal to the injection (brainstem, cerebellum) (FIG. 3A). To identify a suitable control, Htt expression was assessed in these brain regions after ICV delivery of vehicle (0.9% saline) or a non-targeting siRNA conjugated (e.g., SEQ ID NO: 3) to the L2 lipid (termed L2-siRNA^{NTC}, targeting luciferase, which is not expressed in mice). It was determined that treatment with L2-SiRNA^{NTC} did not appreciably change Htt expression compared to vehicle and therefore proceeded with L2-siRNA^{NTC} as the primary negative control for gene silencing studies.

At 1 month after injection, Htt-targeting L2-siRNA (referred to as L2-siRNA^{Htt}) demonstrated potent mRNA knockdown (>50%) in all brain regions tested, whereas unconjugated siRNA (referred to as siRNA^{Htt}) only exhibited silencing in the cortex and brainstem, highlighting the benefit of L2 conjugation (FIG. 3B, FIG. 3C, and FIG. 3D). Similarly, at the protein level, siRNA^{Htt} mediates some silencing in the cortex and hippocampus, but less than L2-siRNA^{Htt}, which also demonstrates robust knockdown in the striatum (FIG. 3E. FIG. 3F, and FIG. 3G). At 3 months post-injection, mice receiving siRNA^{Htt} no longer exhibited significant protein knockdown in any regions, and only the hippocampus exhibited a reduction in the target mRNA. In contrast, mice receiving L2-siRNA^{Htt} exhibited sustained gene and protein knockdown; significant reduction in HTT protein was noted at 3 months in the striatum, hippocampus, cortex, and cerebellum. Impressively, at a prolonged 5-month timepoint, L2-SiRNA^{Htt} still achieve mRNA silencing in all examined parenchymal regions. The protein levels were also reduced by L2-siRNA^{Htt} treatment in some regions (striatum, hippocampus), while others had returned to basal expression levels. Unconjugated siRNA^{Htt} was omitted from the 5-month study since its activity was lost at 3 months. Overall, the data show that L2-siRNA^{Htt} potentiates widespread and prolonged gene and protein silencing throughout the CNS compared to the parent siRNA.

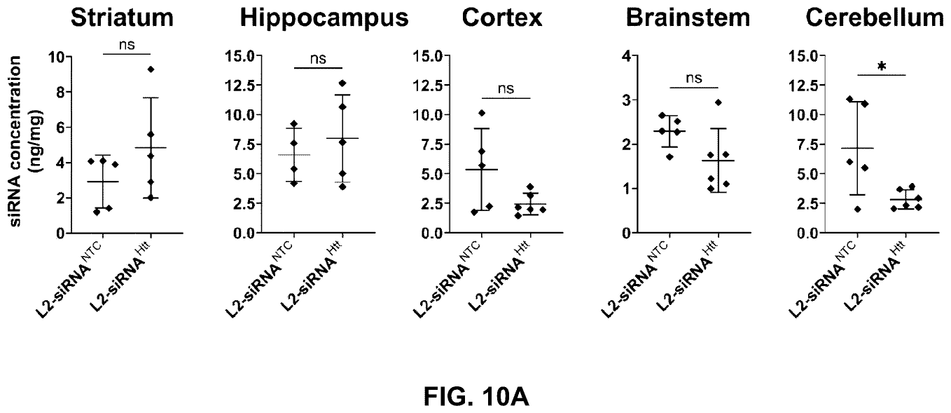

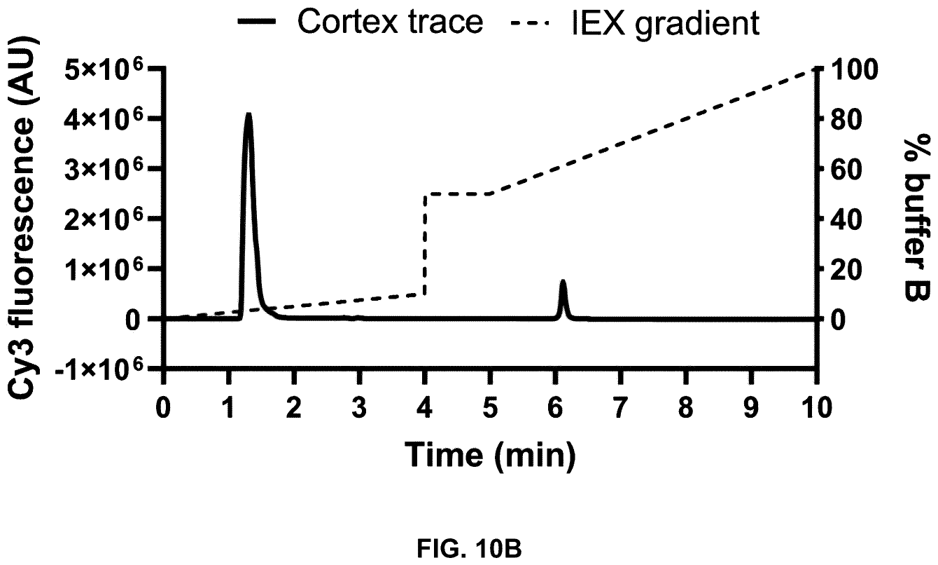

To determine the relationship between siRNA delivery and knockdown, the amount of L2-siRNA present over a time course was examined using a PNA hybridization assay to measure mass of antisense strand per tissue mass (FIG. 3H, FIG. 10A, and FIG. 10B). Consistently, regions with the highest delivery (striatum, hippocampus, and cortex) also potentiated the most silencing activity at all timepoints. While accumulation decreases over time, a considerable amount of siRNA-L2 is still retained at 3 and 5 months. These data suggest that once delivered, this complex remains active in tissue for a prolonged period, a feature that is advantageous for minimizing treatment frequency in the context of managing chronic, genetic, or degenerative neural diseases.

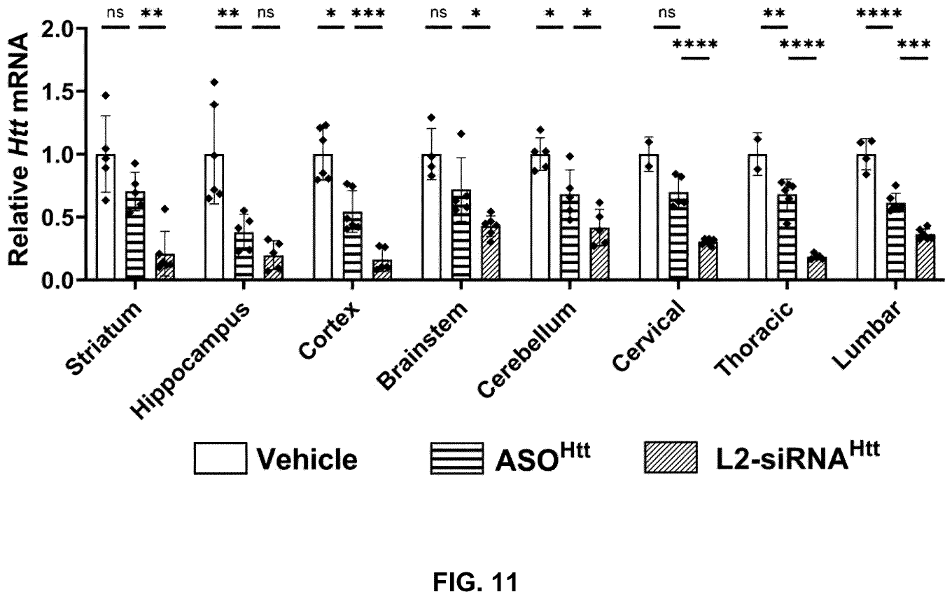

L2-siRNA^{Htt} was also comparted to a Htt-targeting ASO. To match the experimental regime, an equimolar ICV dose (15 nmol, ˜ 95 μg) of the “MoHu” gapmer ASO^{Htt} that was designed with homology for mouse and human HTT was administered. Gene silencing was assessed at a 1-month time point, and given the disparate structures between the two molecules, normalization was performed relative to a vehicle control. L2-siRNA^{Htt} was more potent than ASO^{Htt} given at the same equimolar dose, demonstrating statistically significant enhancement of mRNA knockdown in all parenchyma and spinal cord regions except for hippocampus, where both compounds potentiated robust knockdown (FIG. 11). Of particular note, L2-siRNA^{Htt} was more effective in the striatum (˜80% reduction) than ASO^{Htt} (˜25% reduction), which is highly relevant considering the striatum is the region predominantly affected by Huntington's disease.

L2-siRNA achieves potent gene inhibition comparable to other siRNA conjugates developed for CSF delivery. One such compound is the divalent siRNA, a small (˜25 kDa) and heavily phosphorothioated conjugate that exhibited potent Htt knockdown in mice out to 6 months following ICV delivery of a high dose (40 nmol, ˜2.67× the dose used here to assess L2-siRNA knockdown). In addition, the C16 lipid conjugate developed by Alnylam® Pharmaceuticals utilizes a small hydrophobic lipid in the middle of the siRNA structure as a way to enhance cellular uptake of the siRNA without promoting micellization. While these compounds yield effective gene silencing in many regions, delivery to deep brain structures after intrathecal administration remains a considerable hurdle. Notably, C16-siRNA did not achieve knockdown in the striatum of rats, and the divalent siRNA was not effective throughout the striatum of Dorset sheep. Furthermore, not all cell types examined achieve effective gene silencing, as oligodendrocytes were not effectively silenced with C16. In addition, both L2-siRNA and ASO silence a wide variety of cell types, but in a direct comparison it was demonstrated that L2-siRNA^{Htt} was more potent than dose matched ASO^{Htt} across multiple brain regions, including the striatum where HD pathology manifests.

### Example 12

### L2-siRNA Achieves Durable Gene and Protein Silencing Throughout the Spinal Cord

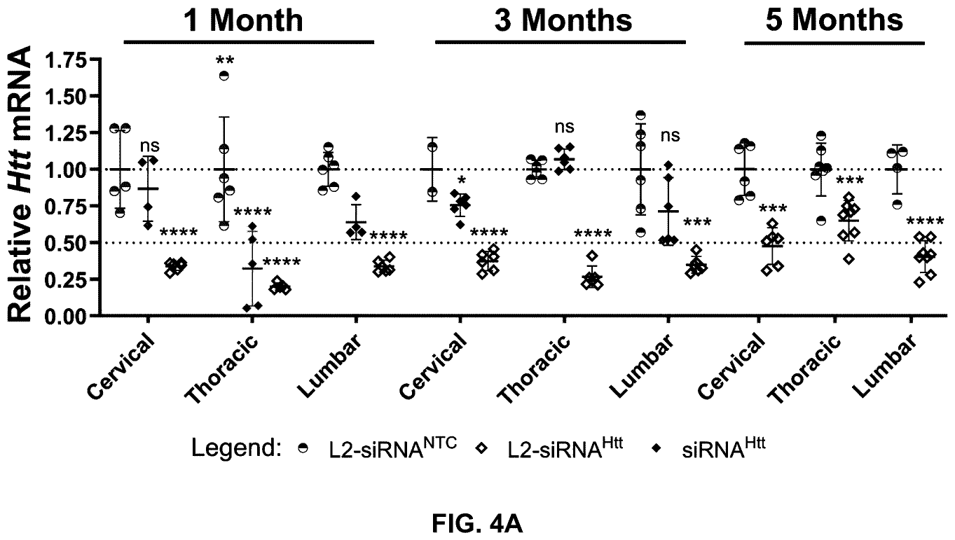

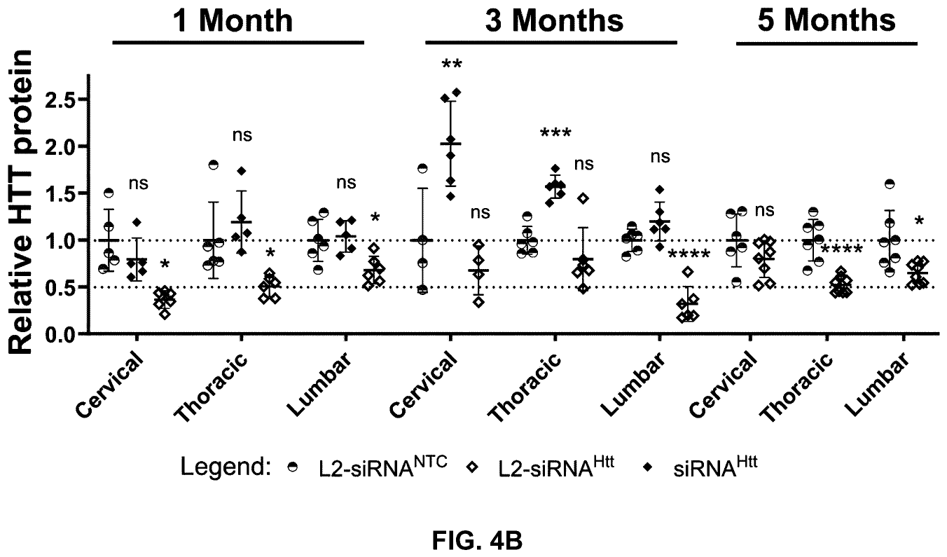

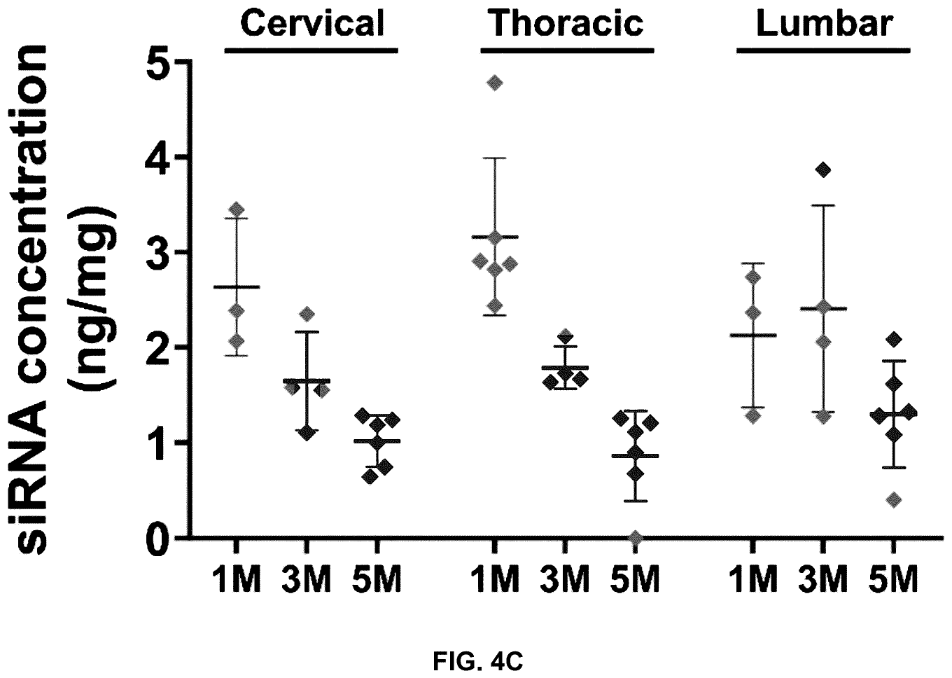

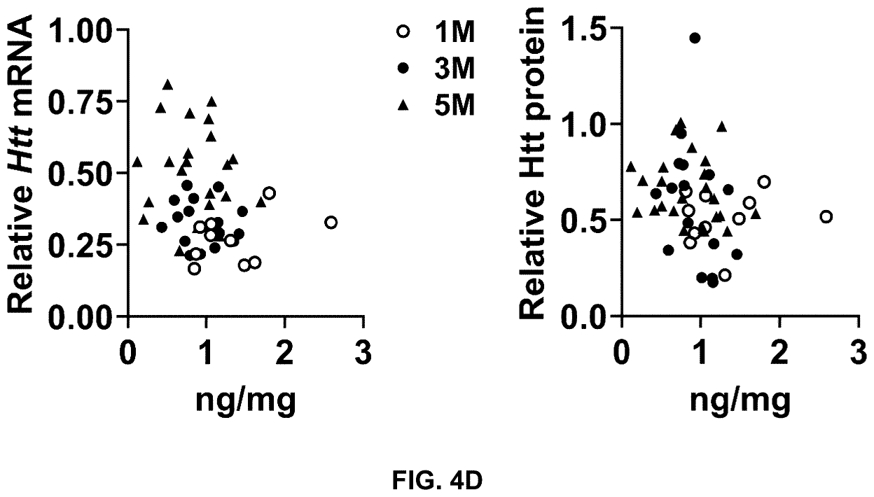

The spinal cord is also directly connected to CSF circulation and implicated in several diseases targetable by nucleic acid therapies. However, unlike for the intrathecal route of administration often used to modulate disease targets of the spinal cord, delivery to the spinal cord requires greater transport from an ICV injection site. Evaluation of the cervical, thoracic, and lumbar spinal cord regions at 1-, 3-, and 5-months post-injection by the PNA assay showed that L2-siRNA levels in the spinal cord decrease linearly over time from ˜3 ng/mg to ˜1 ng/mg (FIG. 4C and FIG. 4D). These levels are lower than the parenchyma, however, L2-siRNA^{Htt} gene silencing activity remained high in all segments of the spinal cord at both the mRNA and protein levels (FIG. 4A and FIG. 4B). These outcomes further highlight the effectiveness of L2-siRNA throughout the CNS.

### Example 13

### L2-siRNA Effectively Targets Diverse CNS Cell Types

Cell type-specific uptake and silencing activity were next rigorously characterized for L2-siRNA. To assess cellular uptake, mice were administered Cy5-tagged L2-siRNA or parent siRNA (10 nmol) by ICV, and flow cytometry was performed after 48 hours to measure Cy5 levels in various cell types defined as: Thy1+ neurons, ACSA2+ astrocytes, CD11b+ microglia/macrophages, and CD31+ endothelial cells. Oligodendrocytes were removed from analysis based on O1+ staining, as myelin can promote non-specific binding. It was found that total and cell-specific uptake were increased by L2 conjugation, in agreement with in vitro data. L2-siRNA uptake into neurons was the lowest out of the cells examined, but this may have been due to poor neuron recovery in the dissociation protocol (<2% of total population). Microglia, which are resident CNS phagocytes, showed the highest L2-siRNA uptake.

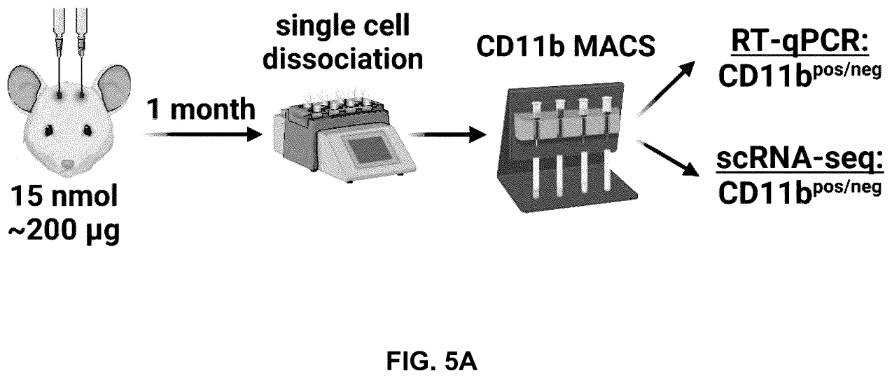

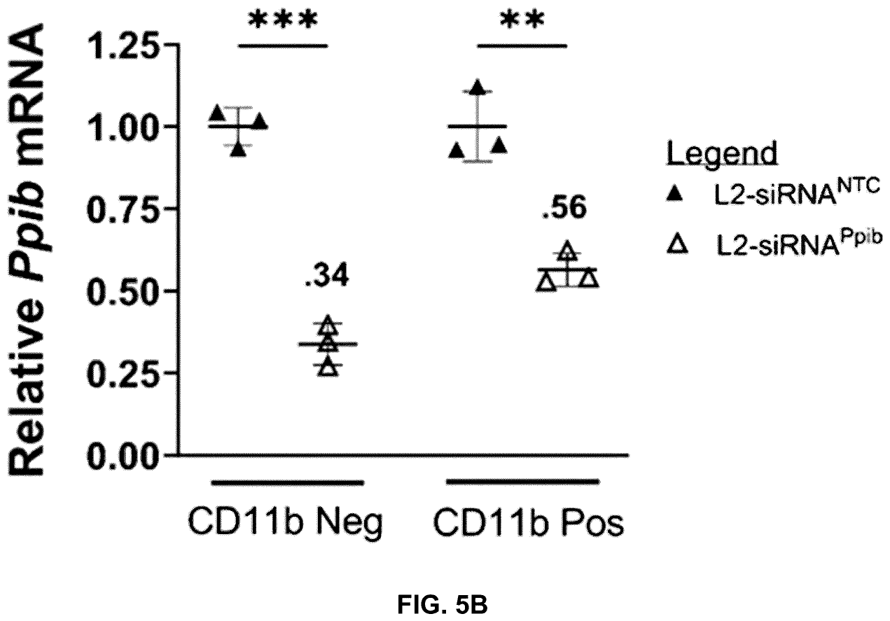

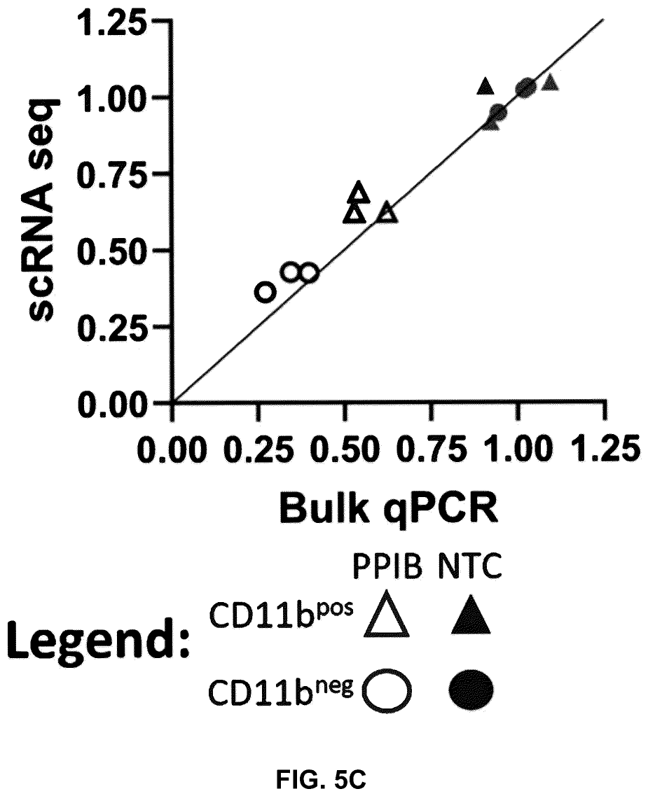

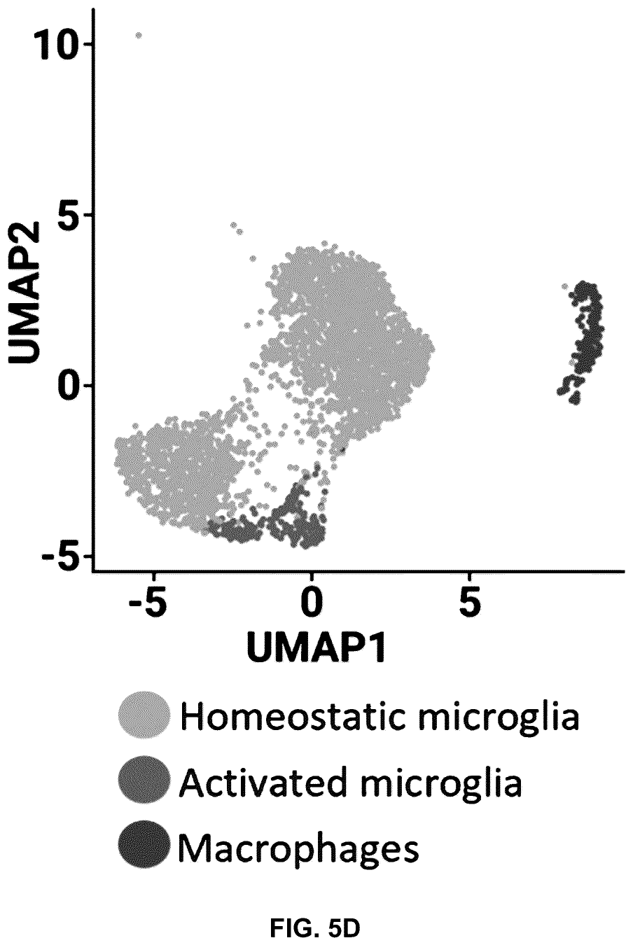

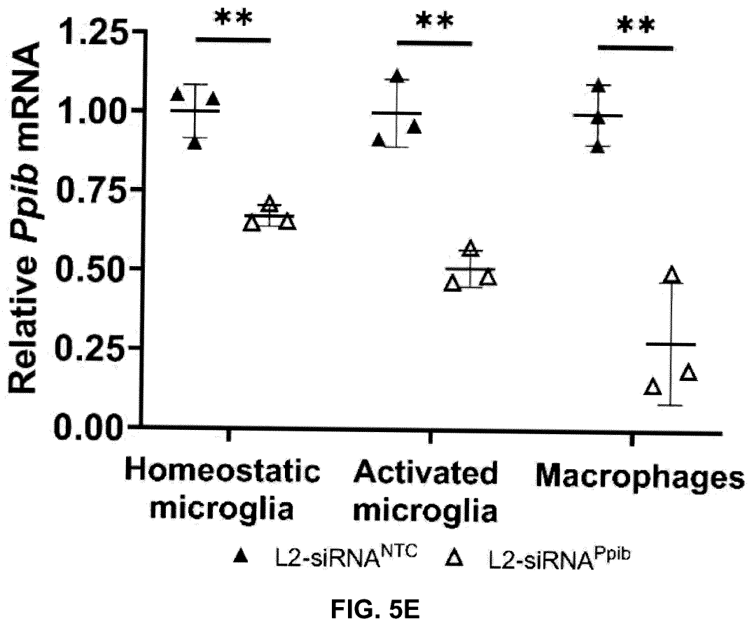

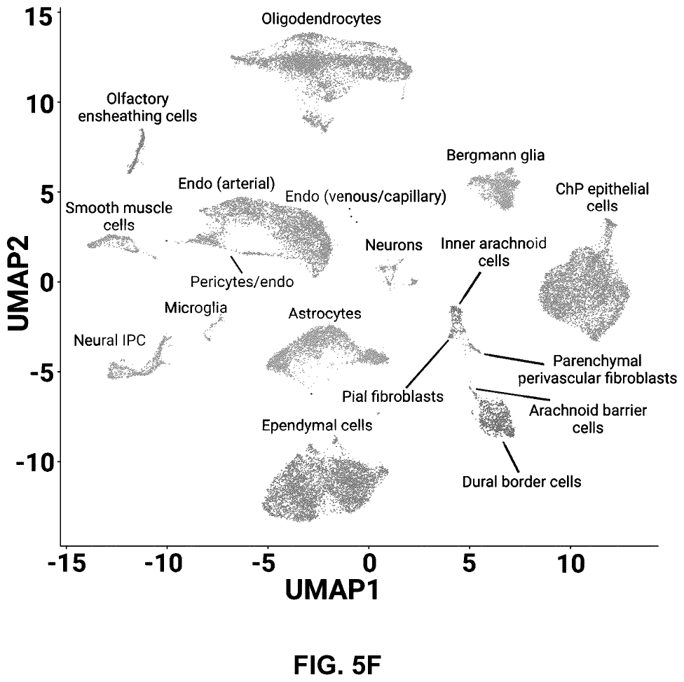

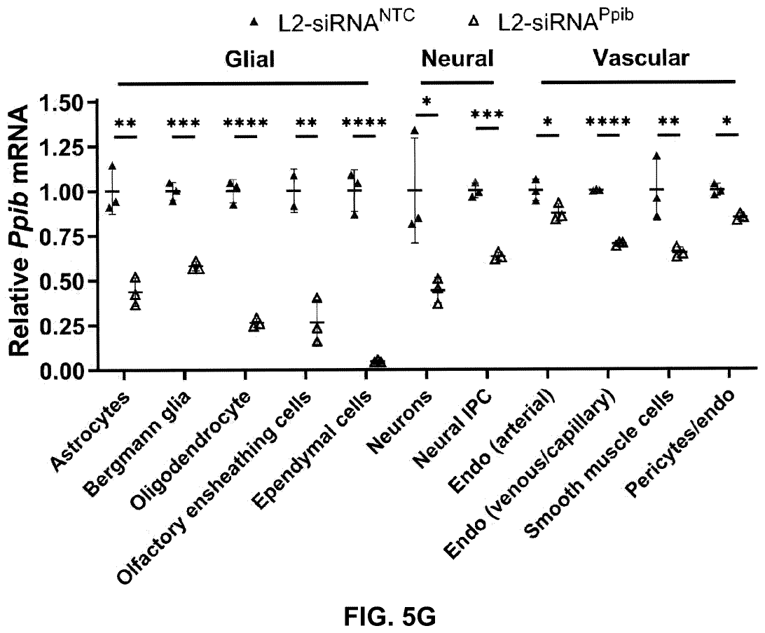

To determine whether L2-siRNA uptake corresponds to functional gene silencing across CNS populations, mRNA knockdown was evaluated using scRNA-seq. Here, an siRNA sequence targeting Ppib was used because this gene is abundantly and ubiquitously expressed across CNS cell types. Highly potent siRNAs have also been designed and validated against Ppib elsewhere, see, e.g., Reynolds, A. et al. Rational siRNA design for RNA interference. Nat. Biotechnol. 22, 326-330 (2004), which is incorporated by reference herein in its entirety. Matching the experimental parameters of bulk knockdown studies, 15 nmol of either L2-SiRNA^{Ppib} or L2-siRNA^{NTC} was injected and collected cells after 1 month for evaluation. Since substantial uptake was observed into Cd11b+ cells by flow cytometry, it was elected to enrich for myeloid cells with bead-based sorting prior to barcoding and analysis, yielding two datasets: Cd11b^{pos} cells encompassing microglia and macrophages, and Cd11b^{neg} cells comprising an untargeted sampling of all other isolated meningeal and parenchymal populations (FIG. 5A). To verify gene silencing with orthogonal methodology, cells not utilized for scRNA-seq were analyzed in parallel using qRT-PCR, which confirmed gene silencing in both Cd11b^{pos} and Cd11b^{neg} populations with an average of 45% and 65% Ppib knockdown, respectively (FIG. 5B). RT-qPCR bulk silencing was compared with Ppib knockdown measured by scRNA-seq and these outputs closely matched for both CD11b^{pos} and CD11b^{neg} populations (FIG. 5C), providing confidence in the accuracy of examining cell-specific gene silencing with scRNA-seq.

To determine gene silencing in myeloid cells, Cd11b^{pos} cells were annotated and clustered into three populations using standard markers (FIG. 5D). Homeostatic microglia are characterized by expression of Crybb1, Cst3, P2ry12, Pros1, and Tmem119, while activated microglia additionally express Apoe, Spp1, Lp1, and Lyz2, see, e.g., Sankowski, R. et al. Mapping microglia states in the human brain through the integration of high-dimensional techniques. Nat. Neurosci. 22, 2098-2110 (2019) and Frigerio, C. S. et al. The Major Risk Factors for Alzheimer's Disease: Age, Sex, and Genes Modulate the Microglia Response to AB Plaques. Cell Rep. 27, 1293-1306.e6 (2019), both of which are incorporated by reference herein in their entirety. CNS macrophages comprise perivascular, meningeal, and choroid plexus macrophages, which are enriched in Pf4, Lyz2, Ms4a7, Ccl24, and F13a1; this population constituted ˜6% of Cd11b^{pos} cells. Compared to the non-targeting control, L2-siRNA^{Ppib} potentiated gene silencing in homeostatic (˜40%) and activated microglia (˜50%), as well as resident CNS macrophages (˜70%) (FIG. 5E). Microglia are notoriously refractory to gene targeting, highlighting the significance of this result. In addition to potent myeloid gene knockdown, L2-siRNA^{Ppib} potentiated gene silencing in a variety of other cell types identified in the Cd11b^{neg} population (FIG. 5F). For example, glial cells such as astrocytes and oligodendrocytes exhibit considerable Ppib knockdown, as well as ependymal cells, which line the ventricles and directly interface with CSF (FIG. 5G). Neurons exhibit modest gene silencing, and due to the low capture of this population, subcluster knockdown into neuron subtypes could not be done. Endothelial cells were subclustered into venous/capillary and arterial populations (FIG. 5F); there was low but significant knockdown in arterial endothelial cells, and slightly higher silencing in the venous/capillary cells (FIG. 5G). Modest gene silencing activity in these cells is consistent with low intensity of uptake observed by flow cytometry. Overall, L2-siRNA^{Ppib} exhibits broad gene silencing activity across diverse CNS cell types, suggesting that it can be leveraged as a platform technology for disease target inhibition.

### Example 14

### L2-siRNA Effectively Targets Brain Border Cells

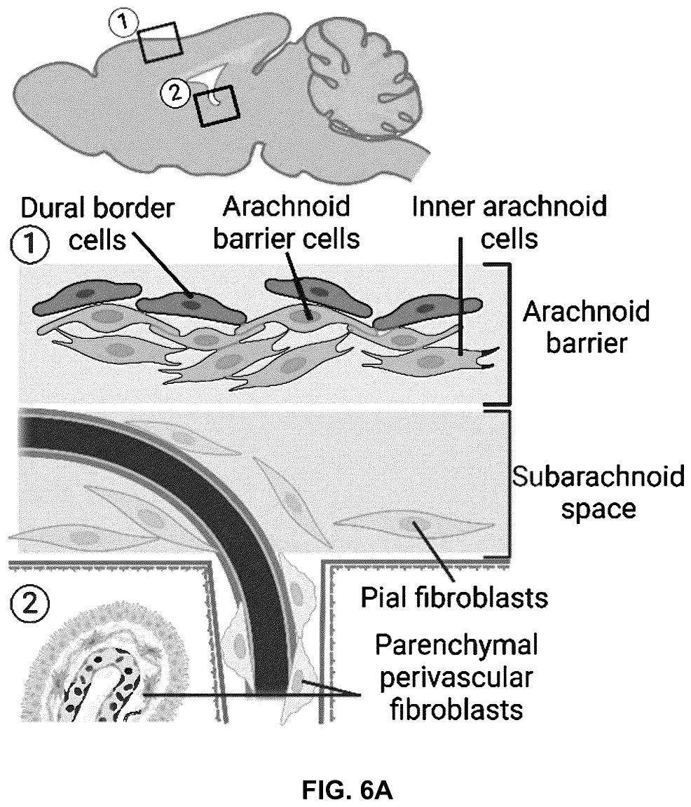

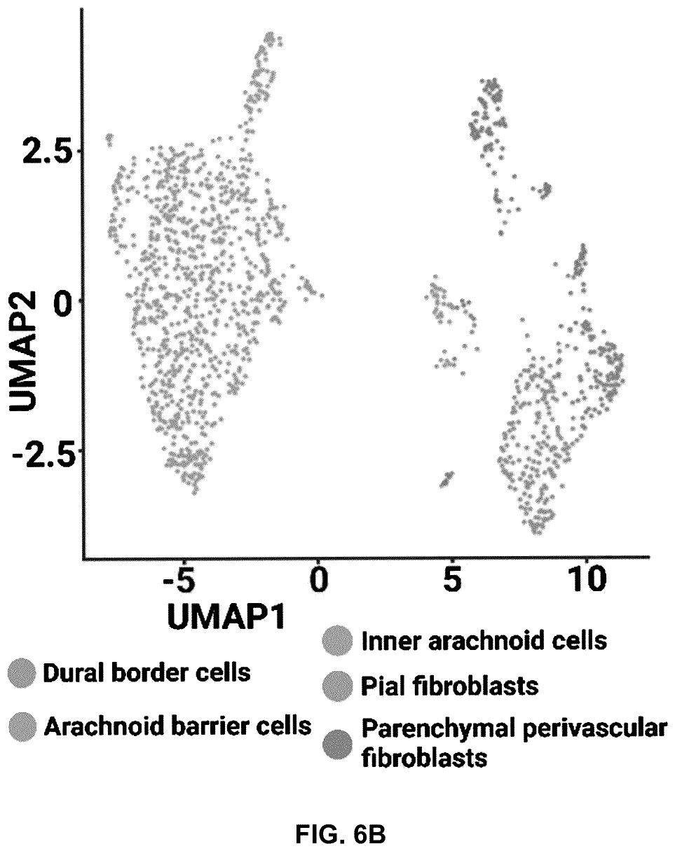

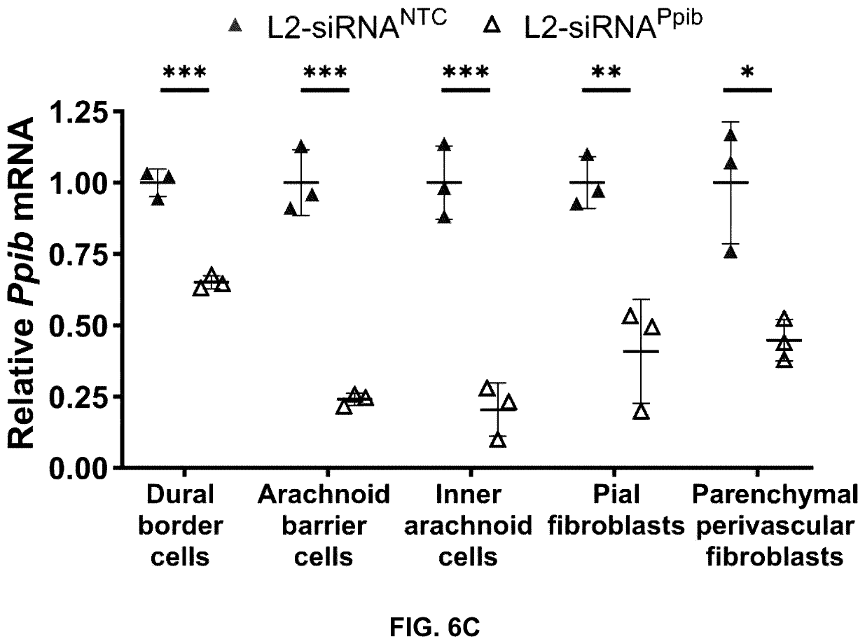

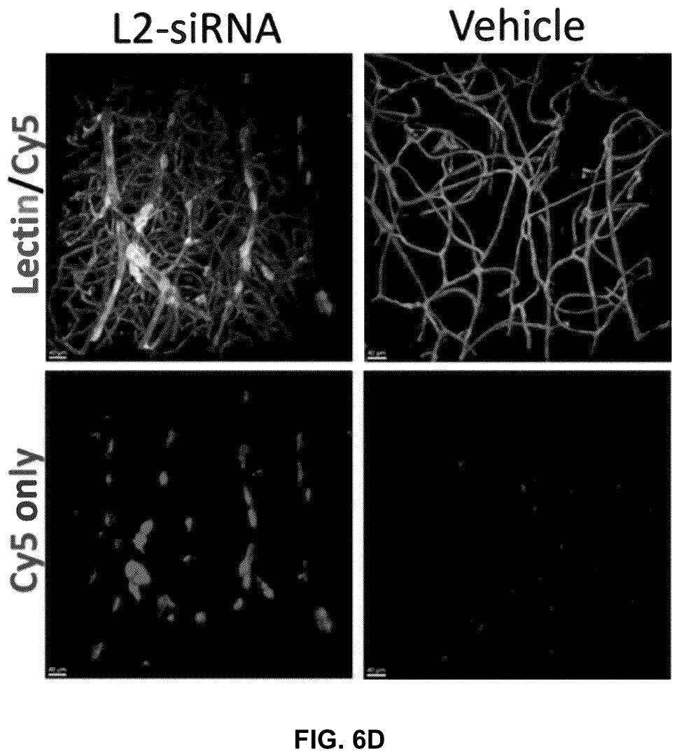

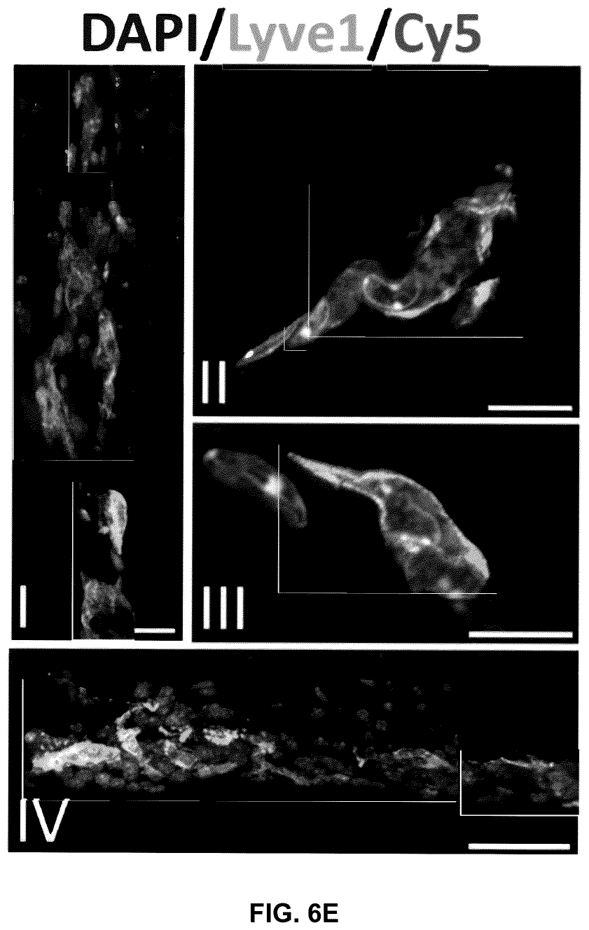

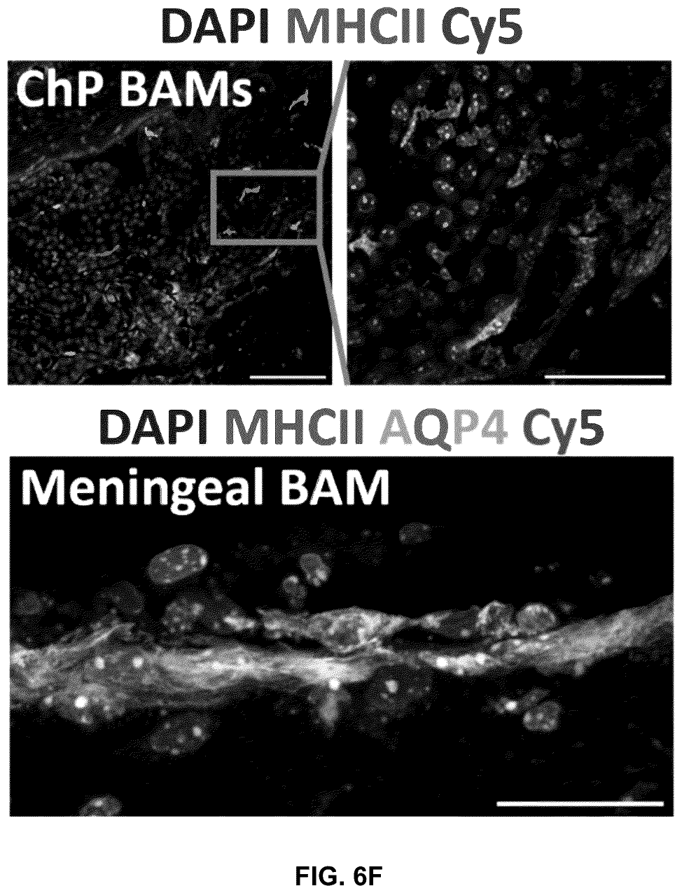

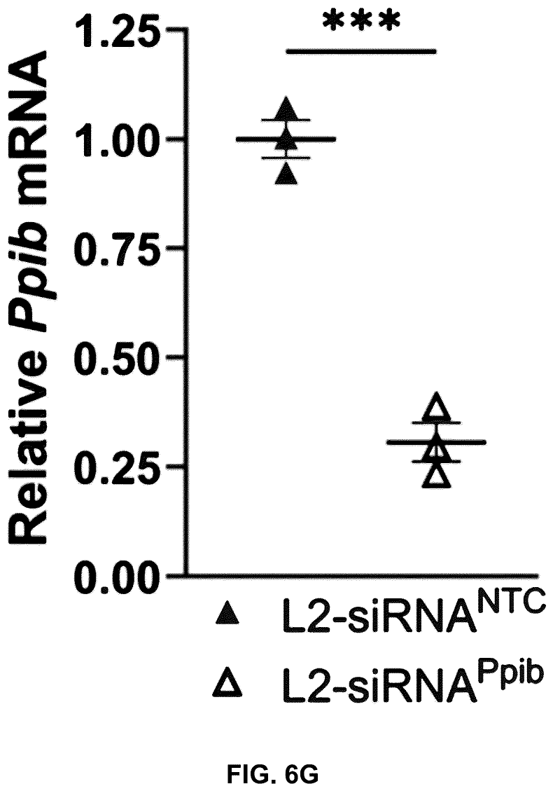

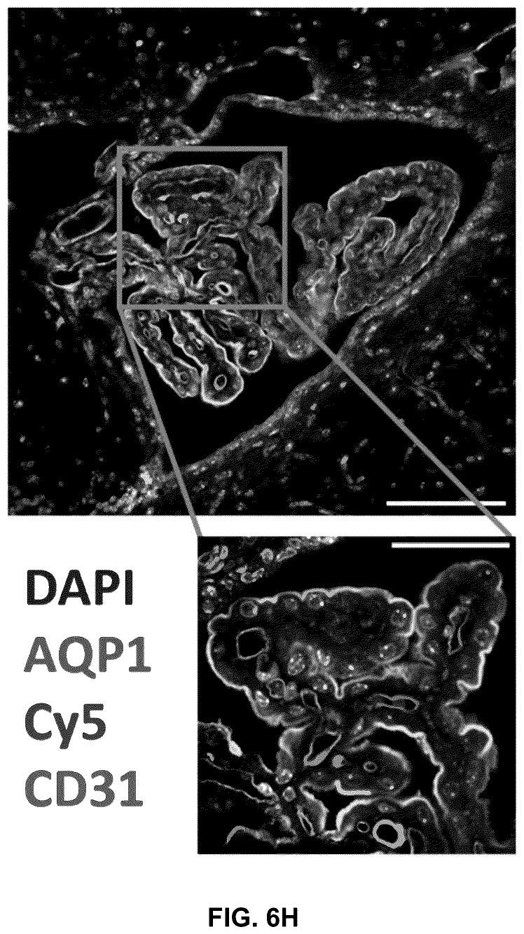

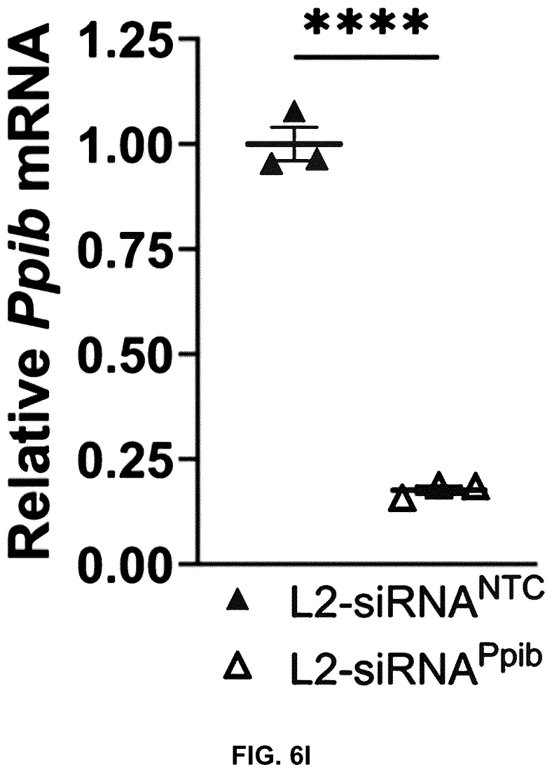

Recent advances in single-cell biology have revealed the diversity of cells present at brain borders including the choroid plexus and meninges, as well as the previously discussed perivascular spaces. There is a growing appreciation for the role brain borders play in disease, but no prior studies have rigorously evaluated siRNA delivery to the cells residing in these borders. Since brain borders are directly connected to CSF flow, L2-siRNA-mediated gene silencing was investigated in these compartments. First, gene silencing was examined in leptomeningeal cells after ICV delivery. Recent characterization of fibroblast-like cells in the meninges has identified five transcriptionally and spatially unique cell populations. Anatomically, the arachnoid membrane separates the dura from subarachnoid CSF and from top to bottom is composed of dural border cells, arachnoid barrier cells, and inner arachnoid cells. Beneath this membrane are pial fibroblasts, either associated with vessels (perivascular) or sitting on the pial membrane (FIG. 6A). The scRNA-seq data set was subclustered to identify the aforementioned cells, annotating all fibroblasts outside the meninges as “parenchymal perivascular fibroblasts”, which includes perivascular, choroid plexus, and potentially median eminence fibroblasts (FIG. 6B). Impressively, knockdown was observed in all fibroblast-like populations, with dural border cells experiencing the least gene silencing, possibly because of their location on the opposing side of the arachnoid membrane. The greatest fibroblast knockdown potency was observed in cells directly contacting CSF, such as arachnoid barrier cells, pial fibroblasts, and arachnoid barrier cells (FIG. 6C).

Border-associated macrophages also reside in CSF outside of the brain parenchyma, primarily in the choroid plexus, meninges, and perivascular spaces, and are central players both in maintaining homeostasis and mediating CNS inflammatory responses. When visualizing the perivascular network with CLARITY and light sheet microscopy, a high concentration of L2-siRNA was observed within perivascular cells throughout the parenchyma (FIG. 6D). To assess the identity of these perivascular cells, macrophages were stained based on recent work that subdivided border-associated macrophages into MHCII+ macrophages, which express genes involved in antigen presentation, and Lyve1+ scavenger macrophages, which perform a variety of essential functions including the regulation of CSF flow dynamics. Extensive delivery to perivascular Lyve1+ macrophages was observed, as evidenced by localization of bright L2-siRNA puncta (FIG. 6E). MHCII+ macrophages are most abundant in the choroid plexus and meninges, and widespread uptake of L2-siRNA was observed in this population (FIG. 6F). To assess knockdown in macrophages, These populations from the L2-siRNA^{Ppib} scRNA-seq knockdown study were subclustered. While the Lyve1+ population was too sparse to evaluate, robust gene silencing was found in the MHCII+ macrophages (FIG. 6G). Lastly, delivery to other cells in the choroid plexus, the structure that produces CSF in the ventricles, was examined and high delivery to choroid plexus epithelial cells corresponding to robust gene silencing was noted (FIG. 6H and FIG. 6I).

Growing evidence suggests that CSF facilitates communication between meningeal compartments and CNS parenchyma, functioning to remove waste products and transport antigens. These functions are mediated by cells residing in brain borders (of either fibroblast or myeloid origin) in both homeostatic and disease conditions. For example, recent studies implicate activation of perivascular fibroblasts as a contributing factor to neurodegeneration at early stages of Amyotrophic Lateral Sclerosis, and meningeal fibroblasts-induced fibrotic scarring has been highlighted as a potential player in the pathogenesis of multiple sclerosis. Here, scRNA-seq was leveraged to further characterize L2-siRNA targeting of CNS fibroblasts by examining gene silencing in these rare yet crucial populations residing within brain borders. Interestingly, L2-siRNA mediated gene silencing in fibroblasts spanning the arachnoid membrane, where knockdown correlated with distance from CSF; more knockdown was observed in inner arachnoid cells and arachnoid barrier cells (˜75%) than dural border cells (30%), suggesting that L2-siRNA can either cross or bypass the arachnoid barrier. There was also interest in targeting of border-associated macrophages as they are key players in a myriad of CNS disorders, including driving neurovascular dysfunction in hypertension and Alzheimer's disease. One recent study identified two subtypes of macrophages, Lyve1+ and MHCII+, and demonstrated that Lyve1+ macrophages decrease with age leading to impaired CSF dynamics that can be restored with administration of macrophage colony-stimulating factor (M-CSF). MHCII+ BAMs are also implicated in neurodegeneration, for example by initiating α-synuclein-mediated neuroinflammatory responses through restimulation of CD4+ T-cells. L2-siRNA accumulates in both type of macrophages, and robust gene silencing was observed in MHCII+ macrophages.

### Example 15

L2-siRNA does not Induce Hallmarks of Toxicity

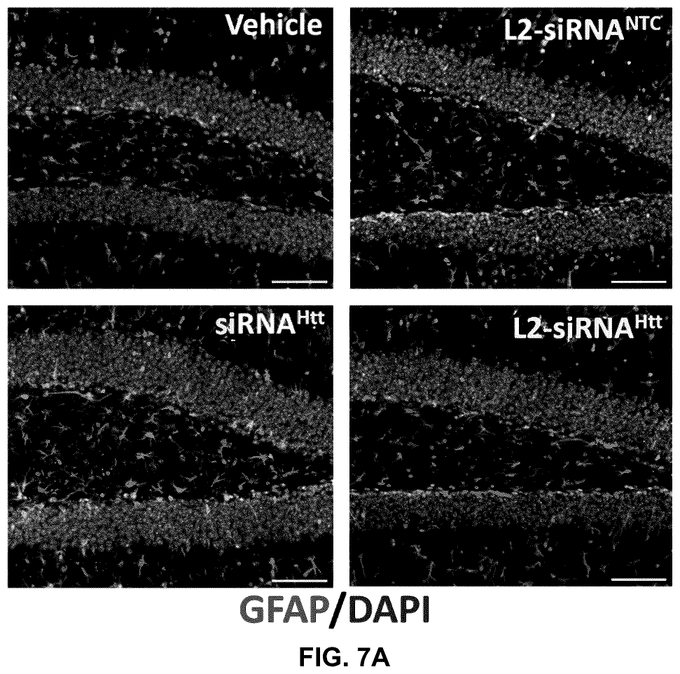

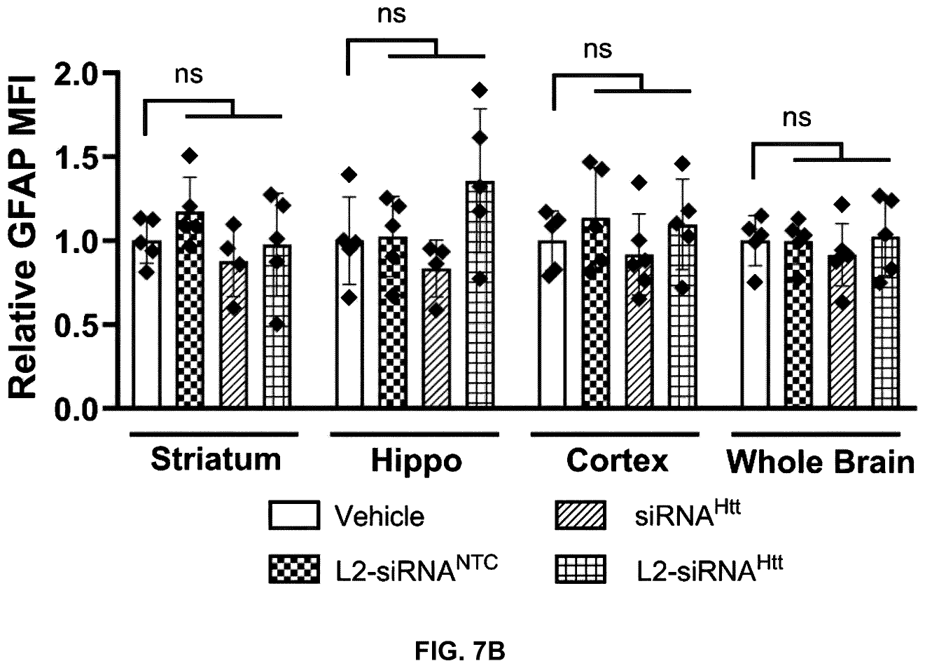

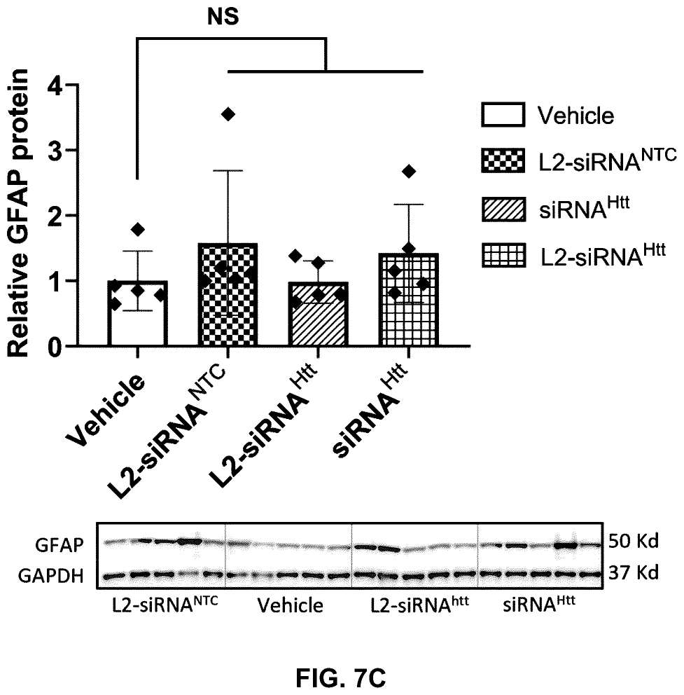

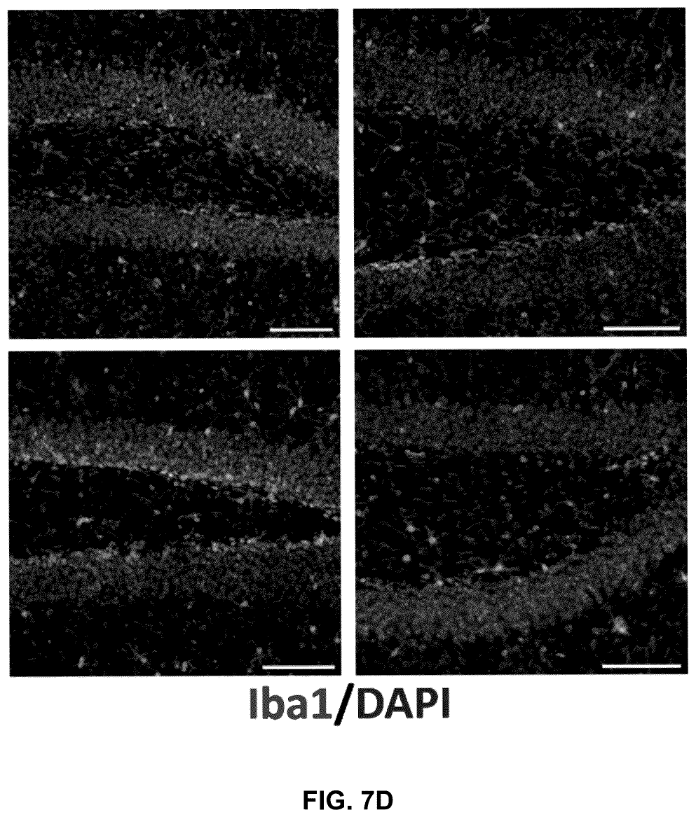

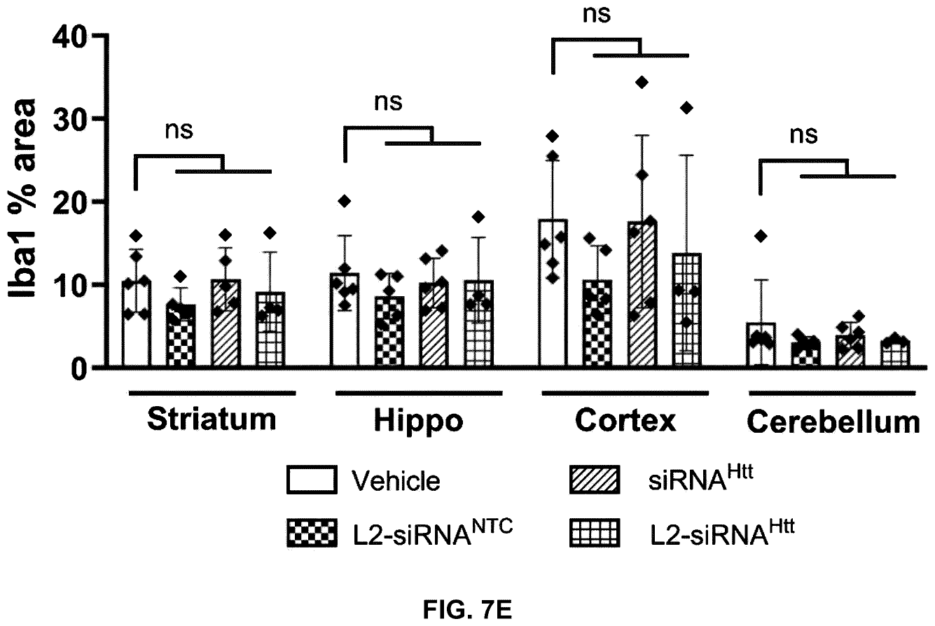

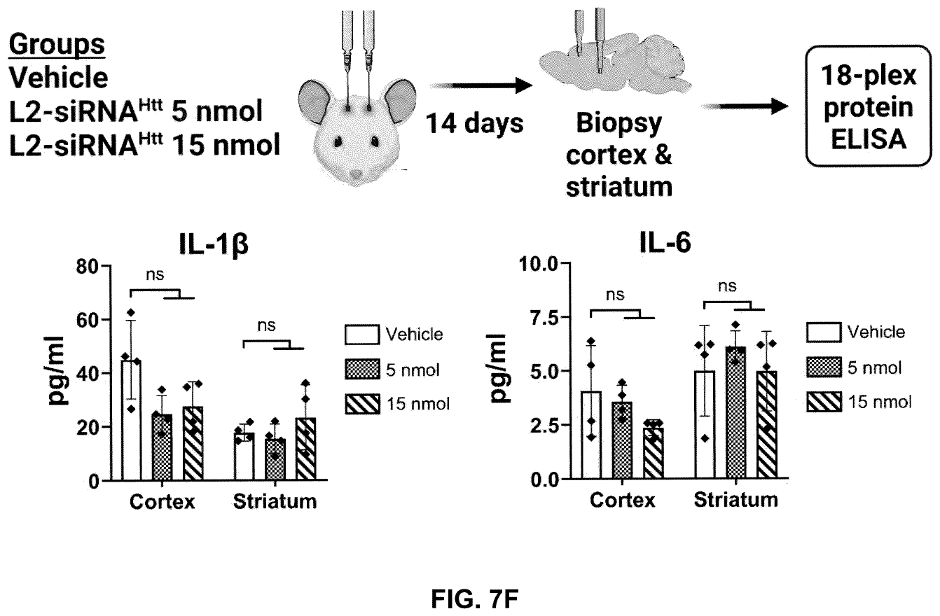

To determine whether L2-siRNA is well-tolerated, several hallmarks of potential adverse reactions were examined, including reactive astrogliosis, neuroinflammation, and systemic organ damage. Overall, no visible signs of toxicity after surgery were observed and every mouse injected with an L2-siRNA conjugate (Htt, Ppib, NTC) survived to their predetermined endpoint. Astrogliosis is a multi-faceted process through which astrocytes respond to damage or disease and is characterized by elevated GFAP expression, indicative of cytoskeletal hypertrophy. Immunohistochemical staining of GFAP 1 month after ICV injection did not show any differences between L2-siRNA groups (NTC, Htt) and vehicle (FIG. 7A and FIG. 7B). These results are consistent with Western blot quantification of GFAP in the hippocampus (FIG. 7C), collectively suggesting that L2-siRNA does not induce astrogliosis.

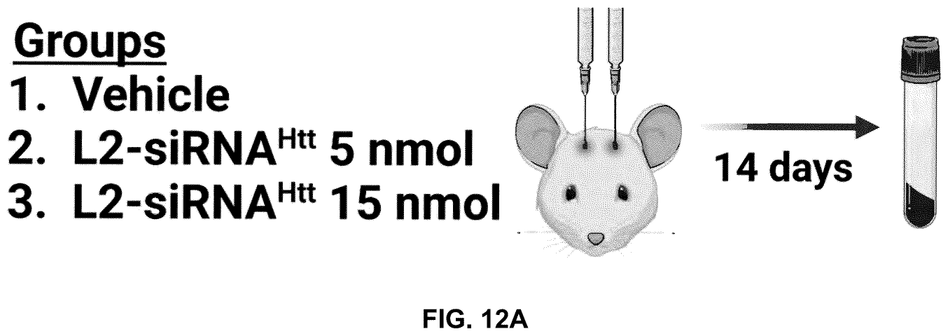

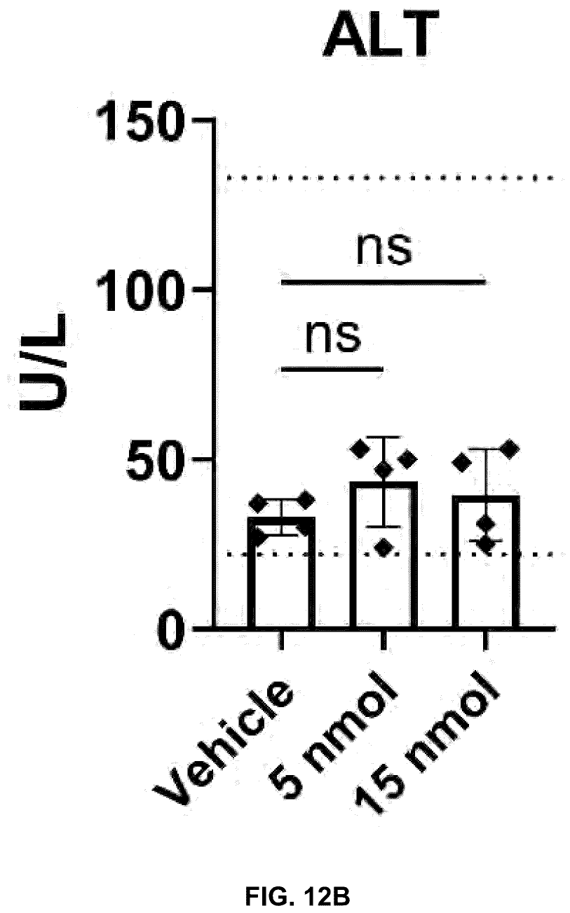

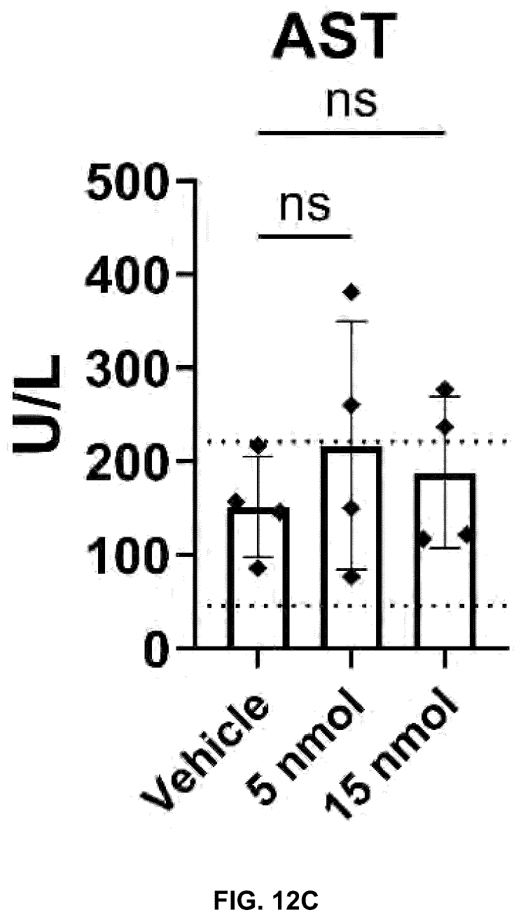

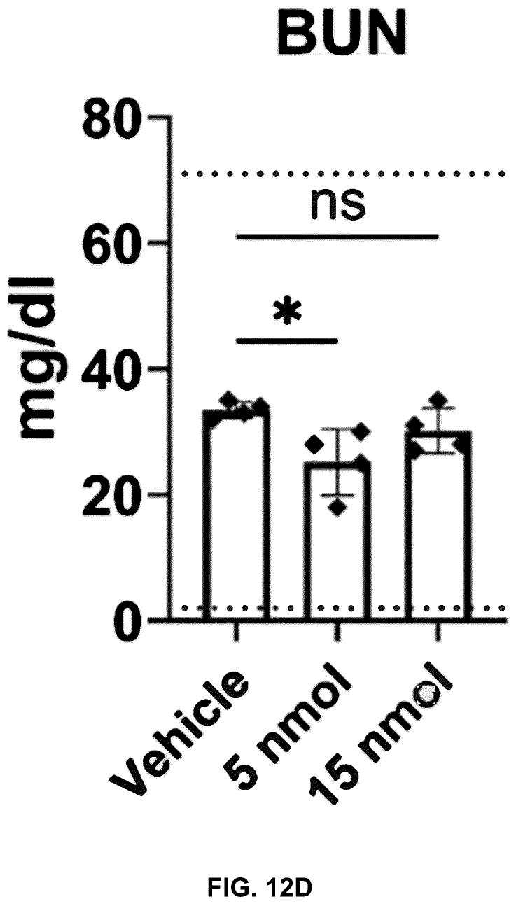

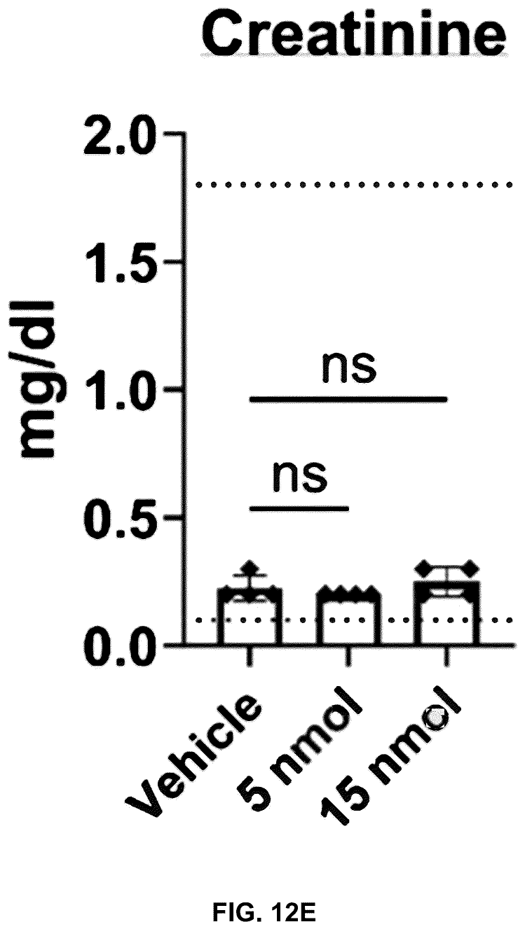

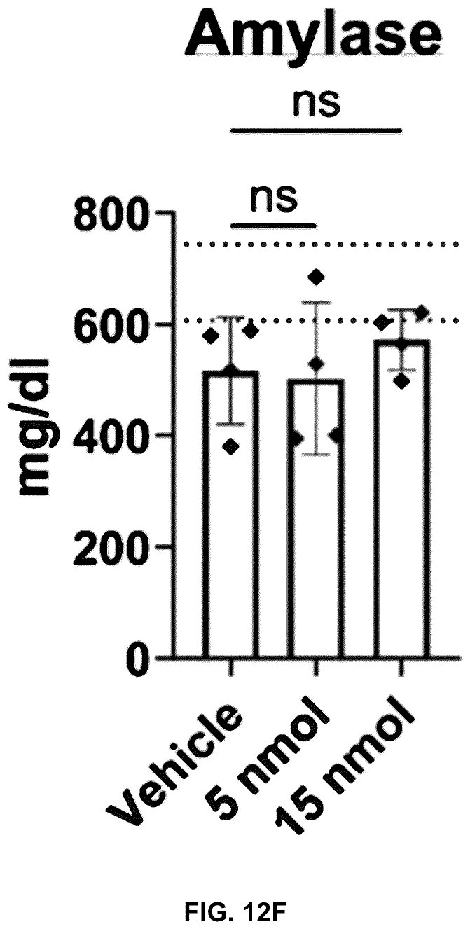

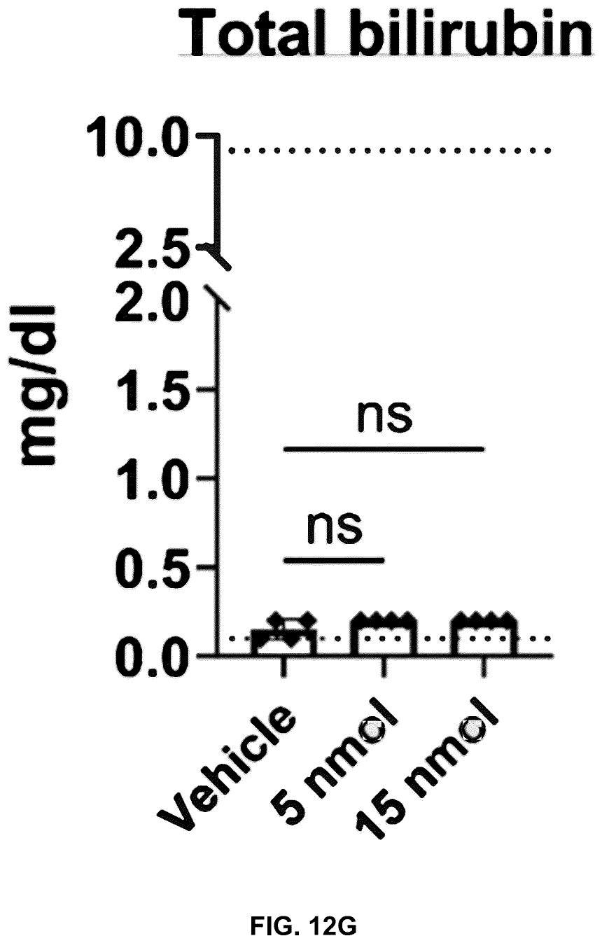

Next, whether ICV injection of L2-siRNA induced any neuroinflammatory responses was investigated. Microglia activation was assessed by Iba1 expression, indicative of a proliferative phenotype. Iba1 protein levels were unchanged after L2-siRNA injection compared to vehicle (FIG. 7D and FIG. 7E). To more broadly examine CNS inflammation, an ELISA panel was utilized to evaluate cortical and striatal tissue two weeks after ICV injection, at either the standard dose used in knockdown studies (15 nmol) or a lower dose (5 nmol). Notably, no difference in pro-inflammatory cytokine, chemokine, or growth factor levels were observed between vehicle and L2-siRNA^{Htt} (FIG. 7F). Changes in serum markers after ICV injection were not seen (FIG. 12). Collectively, L2-siRNA did not exhibit detectable dose-limiting toxicities, suggesting that L2-siRNA is a safe drug conjugate.

In summary, the Examples suggest that L2-siRNA possesses unique properties that enhance its transport and brain-wide distribution relative to other conjugates, thus yielding a promising platform technology for silencing genes implicated in CNS disorders.

### Methods for Examples 16-17

Intravenous Injections of siRNA Compounds

On the day of injection, the siRNA duplexes were annealed in 0.9% sterile saline by heating to 95° C. and gradually cooled to 4° C. If necessary, samples were concentrated with a 3K Amicon Ultra spin filter (UFC500324). To perform intravenous injections into the tail vein, the mice were restrained and siRNA or L2-siRNA was slowly injected into one of the lateral tail veins.

At the terminal timepoint, mice were euthanized by ketamine (450 mg/kg)/xylazine (50 mg/kg) overdose and transcardially perfused with cold heparinized (10 U/ml) DPBS−/− to remove blood cells from the vasculature. For biodistribution studies, mice were further perfused with 4% paraformaldehyde (PFA), and then brains, spinal cords, and organs were extracted, immersion fixed in 4% PFA overnight at 4° C., and subjected to further downstream histological processing. For gene silencing studies, the choroid plexuses were manually dissected and the remainder of the brain used for CD31 MACS sorting.

### MACS Endothelial Isolation

After harvesting the brain, it was digested into a single cell suspension using the papain-based mouse adult brain dissociation kit from Miltenyi Biotec (130-107-677). Manufacturer instructions were then followed to perform CD31 MACS bead sorting. In brief, 10 μl of beads (Miltenyi 130-097-418) were added to 90 μl of cells and incubated for 15 minutes at 4° C. After washing with PB buffer (DPBS+0.5% BSA), cells were resuspended in 500 μl of buffer and magnetically separated using an MS column

Flow cytometry was performed identically to the methods describing the ICV study, except that the samples were run on a BD LSR Fortessa flow cytometer.

### Histology

Performed as described in methods for CSF delivery. Additional antibody used was a pan-laminin marker (Novus NB300-144, 1:500).

### Example 16

### L2-siRNA Accumulates in Brain Endothelial Cells and Choroid Plexus Cells

Brain barriers such as the blood-brain barrier (BBB) and blood-CSF barrier (BCSFB) control the exchange of molecules and cells between the blood and central nervous system (CNS). Yet, this meticulous control over material transfer poses a considerable challenge for efficacious delivery of intravenous therapies, spurring decades of research into CNS drug delivery. Indeed, most current brain-targeting strategies have been designed to deliver drugs across brain barriers and no strategies have been developed to target and retain biological cargos within brain barrier cells for therapeutic intervention. Here, the goal was to therapeutically target brain endothelial cells and choroid plexus cells, which compose the BBB and BCSFB, respectively. As the primary site of immune cell extravasation, the ability to modulate gene expression in these cells presents an exciting opportunity for clinical intervention. To that end, a lipid-siRNA conjugate was developed that is delivered to ˜100% of brain endothelial cells and all choroid plexus structures after intravenous administration. It was further shown that a single 20 mg/kg dose of the disclosed conjugate achieves 50% gene silencing in brain endothelial cells (CD31^{pos} sorted) that is sustained for several weeks, while also achieving statistically significant knockdown in manually dissected choroid plexus over a one-month period.

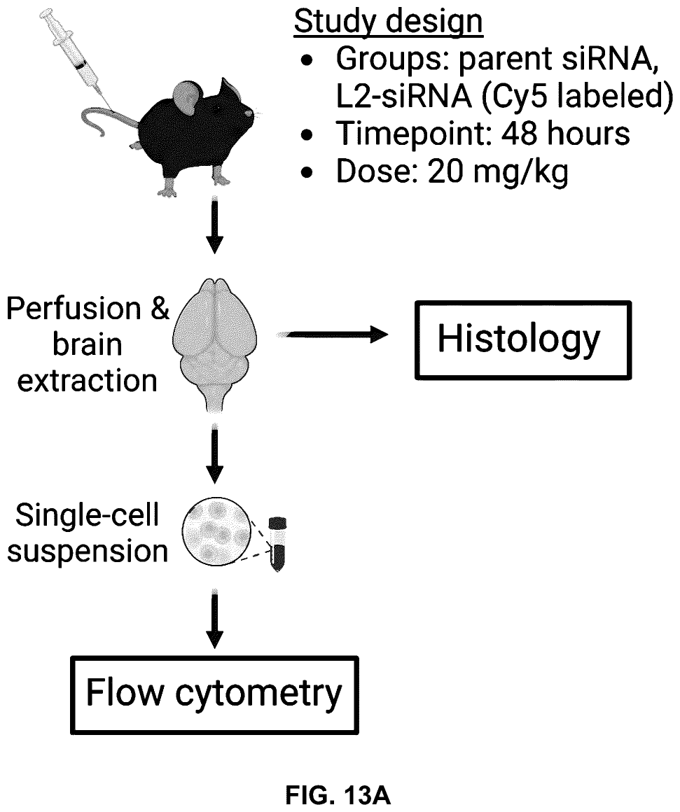

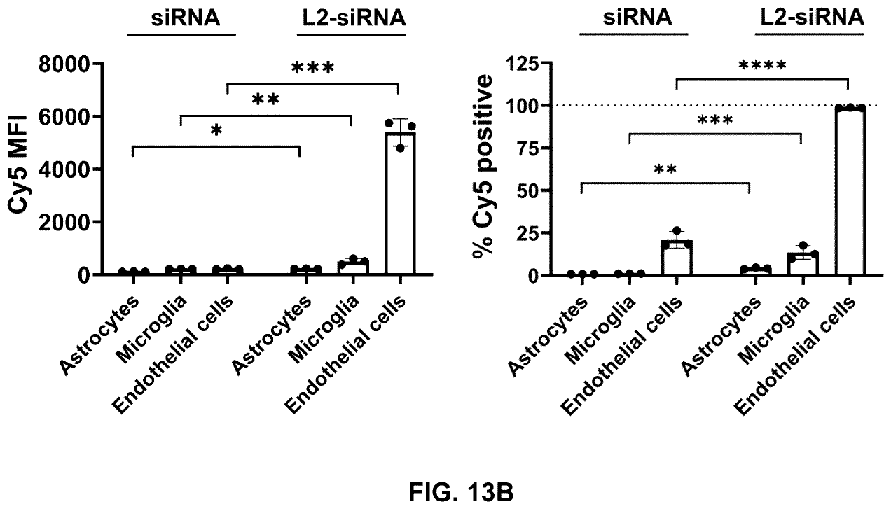

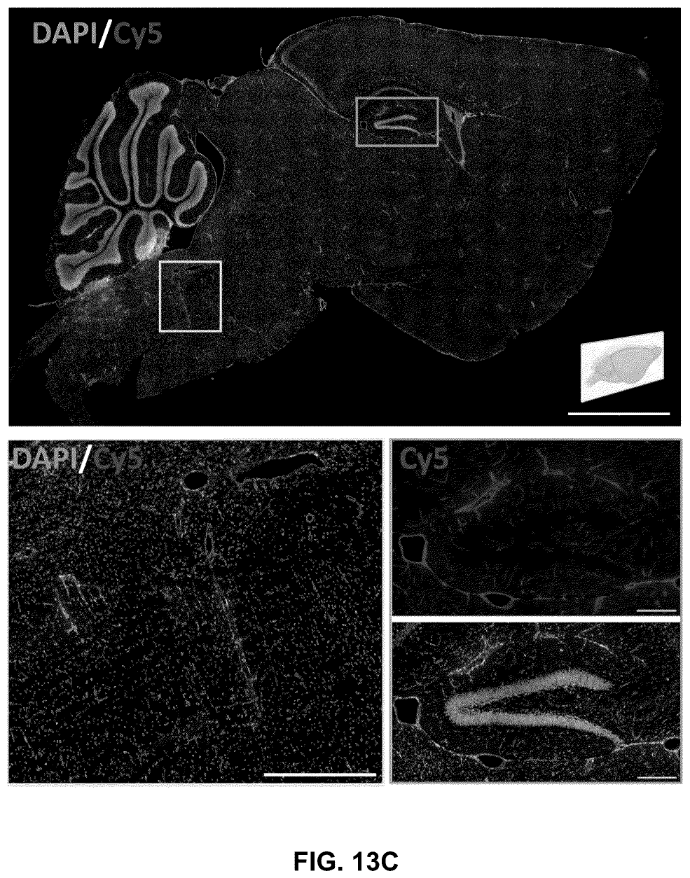

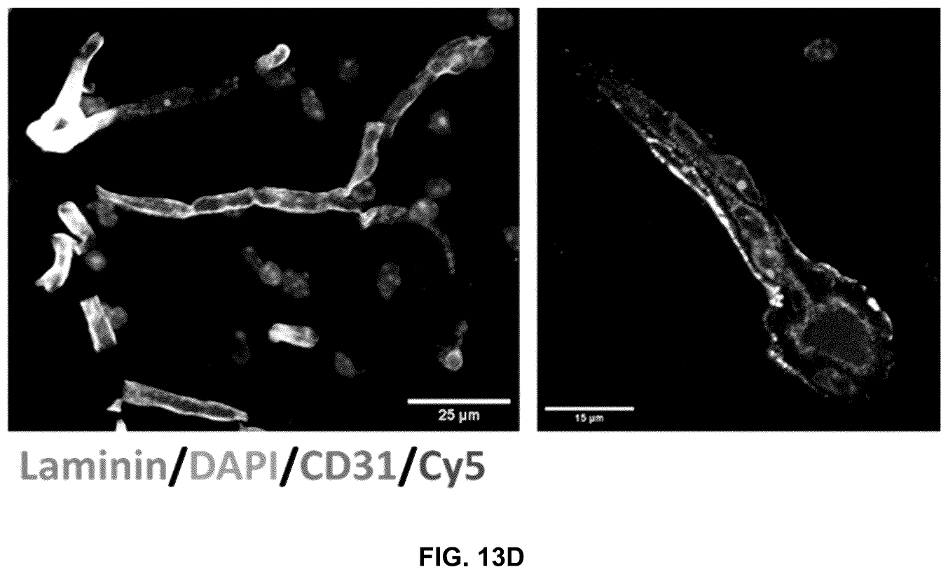

Whether L2-siRNA facilitates delivery to the brain endothelium and choroid plexus was investigated. 20 mg/kg of Cy5 labeled L2-siRNA was injected and after 48 hours cell-specific uptake was quantified with flow cytometry and CNS distribution was visualized with histology (FIG. 13A). To isolate the role of L2 lipid conjugation in promoting distribution, a comparison to unconjugated siRNA was provided, which contains the identical parent “zipper” siRNA sans lipidation. Impressively, the data show that a single intravenous dose of L2-siRNA leads to siRNA uptake in ˜100% of brain endothelial cells, in contrast to the unconjugated siRNA which was taken up into a mere ˜25% of brain endothelial cells (FIG. 13B). These findings are further corroborated by histology, where L2-siRNA distributes in a vascular-like pattern throughout the entire parenchyma (FIG. 13C). To confirm the vascular localization of L2-siRNA, the basement membrane and endothelium were stained with laminin and CD31, respectively. The punctate L2-siRNA signal appears confined to the CD31+ endothelium, with minimal penetration into the basement membrane and parenchyma that surrounds all vessels. (FIG. 13D) Collectively, these data highlight widespread accumulation of L2-siRNA in the brain endothelium after intravenous delivery.

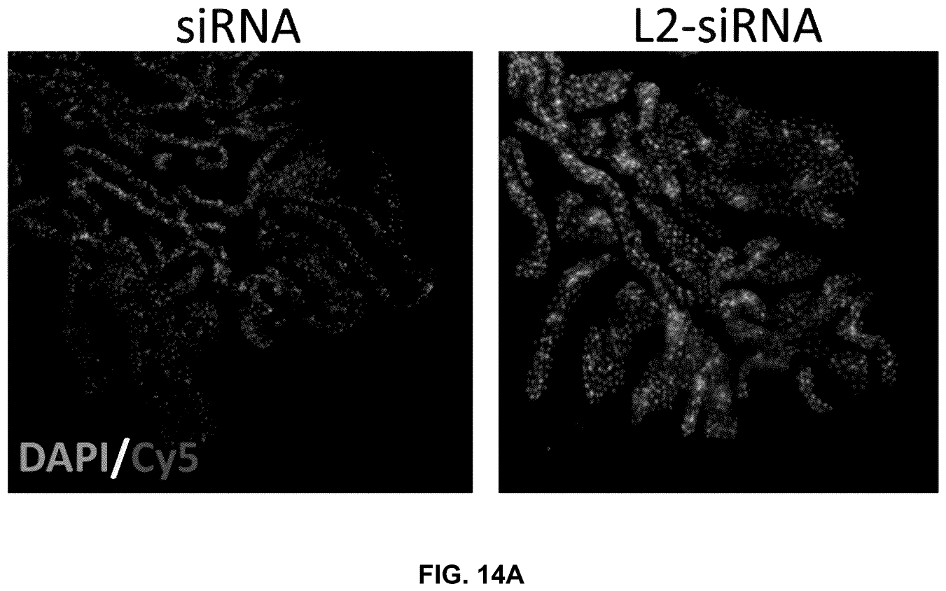

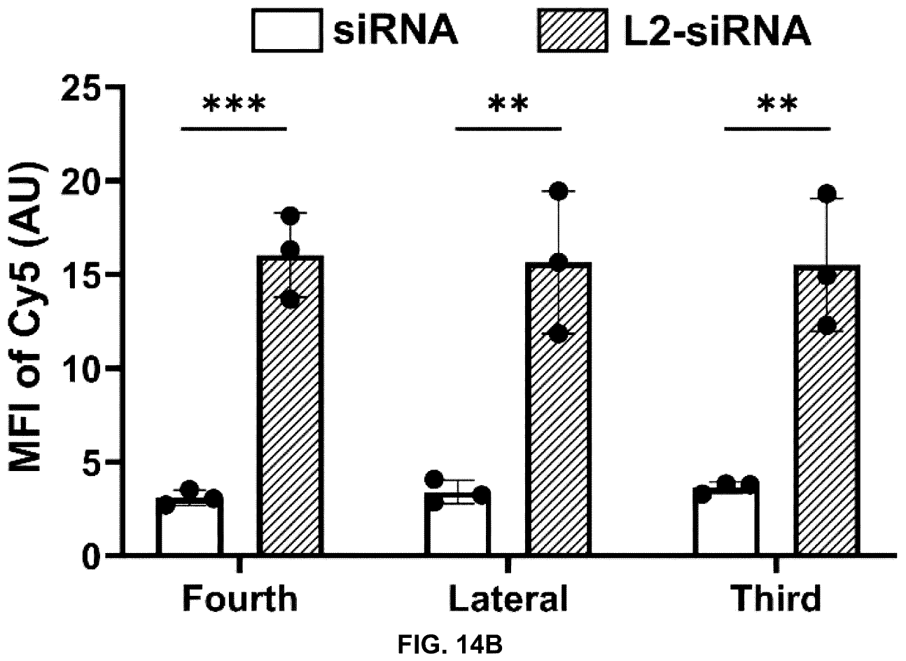

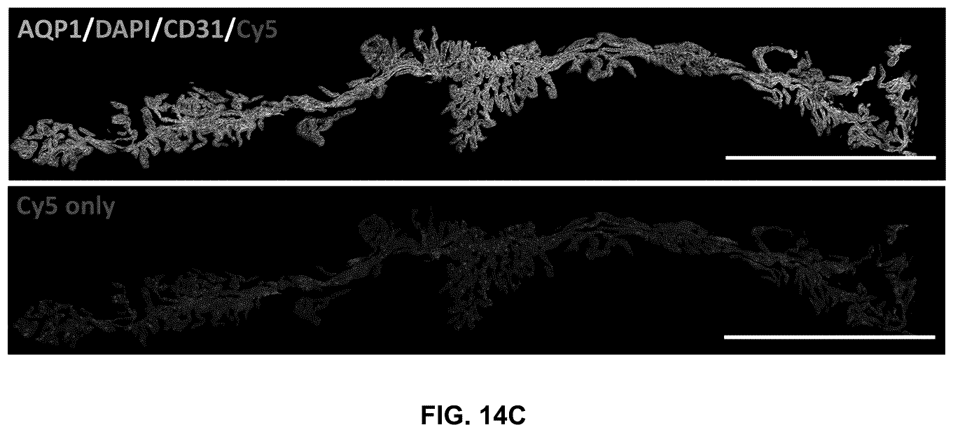

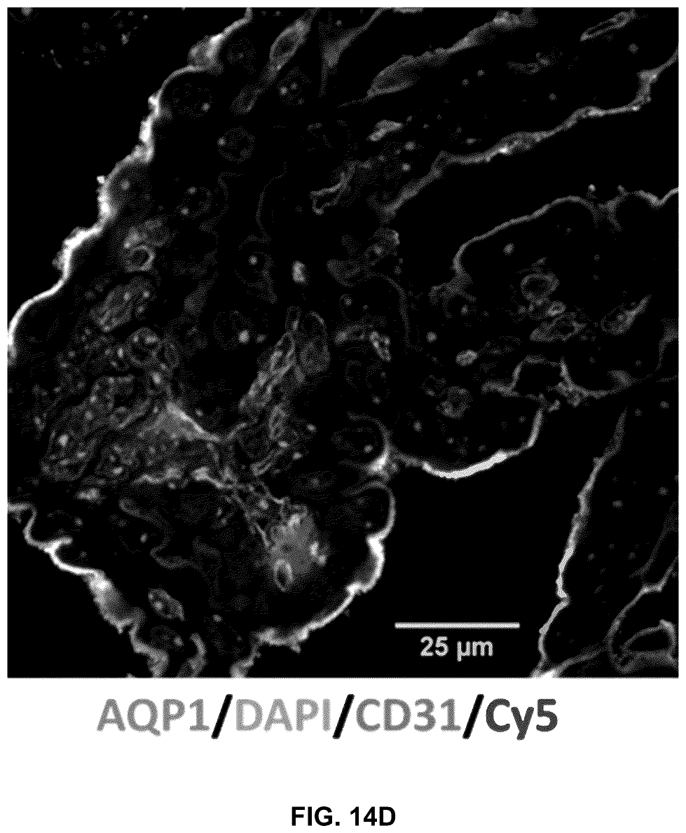

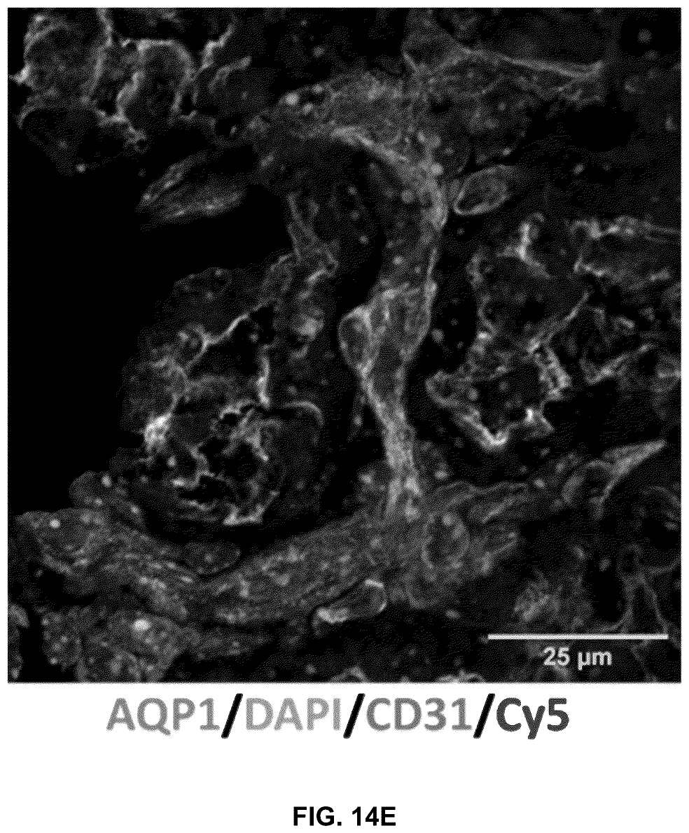

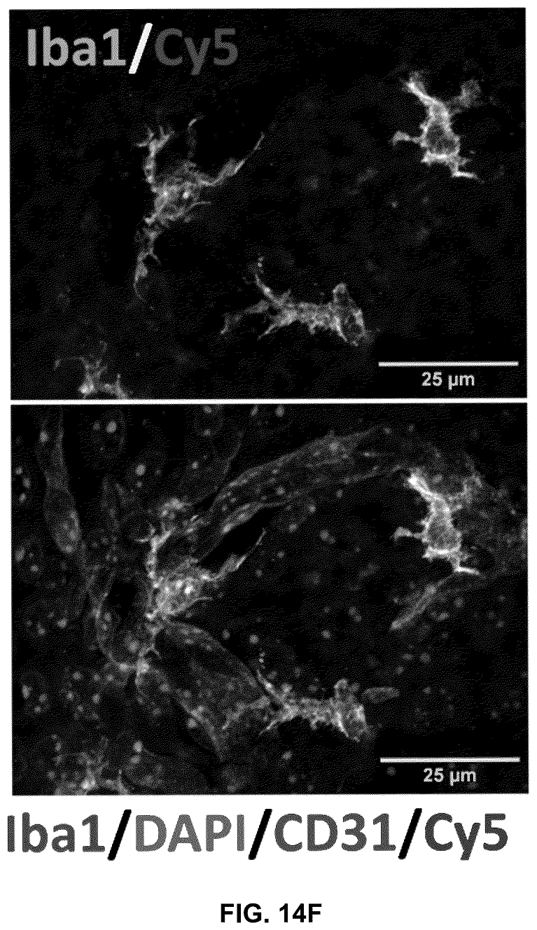

Under the same experimental conditions, delivery to the choroid plexus was assessed, which is composed of a fenestrated endothelium surrounded by a stromal space and continuous columnar epithelium. Compared to unconjugated siRNA, L2-siRNA achieved greater delivery to all choroid plexuses (FIG. 14A and FIG. 14B). Delivery was present throughout the entire structure, as demonstrated in a whole mount image of the 4th ventricle choroid plexus (FIG. 14C). Next, cell-specific staining was employed to examine localization more closely within the choroid plexus. Zooming in, L2-siRNA can be visualized in the stromal space, which lies between the endothelium (CD31+) and epithelium (AQP1+) (FIG. 14D). Further, the punctate pattern of L2-siRNA on the endothelium suggests uptake or transport in choroid plexus vessels (FIG. 14E). Lastly, to investigate which cell types were achieving the greatest uptake (presenting as bright signal in the stromal space), choroid plexus macrophages were stained using Iba1. It was found that these macrophages exhibited considerable uptake (FIG. 14F).

### Example 17

### L2-siRNA Promotes Sustained Gene Silencing in Brain Barrier Cells

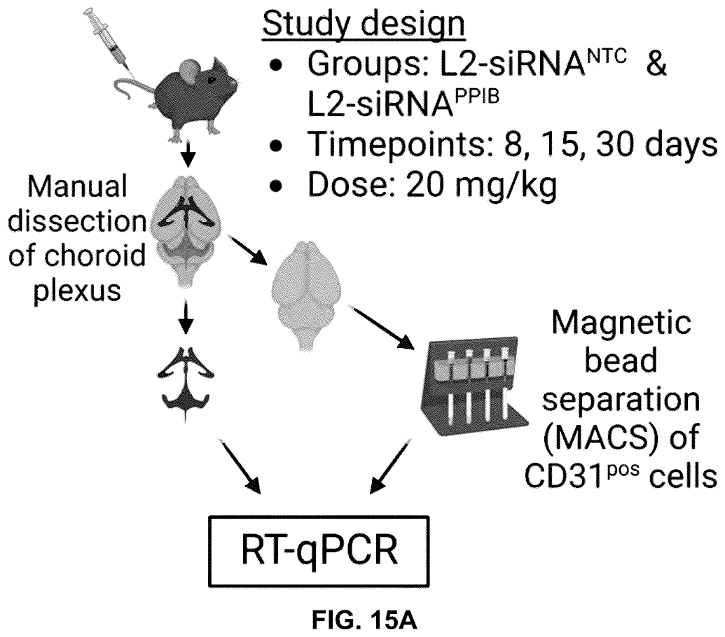

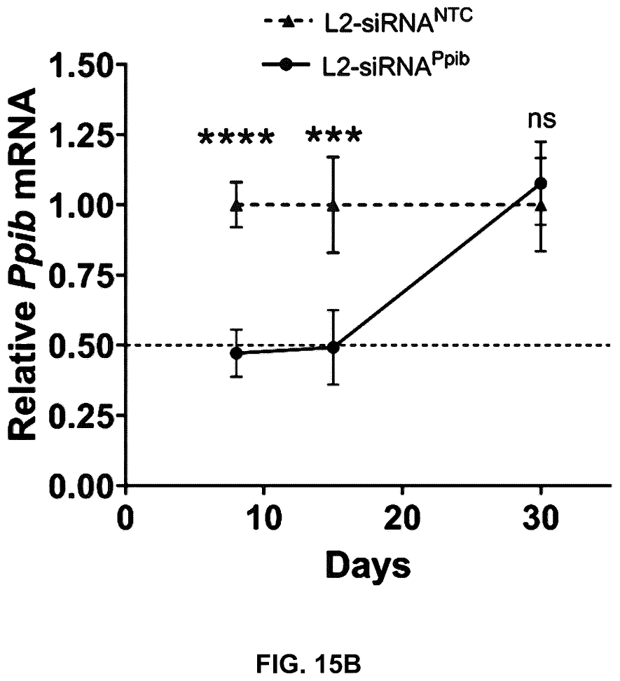

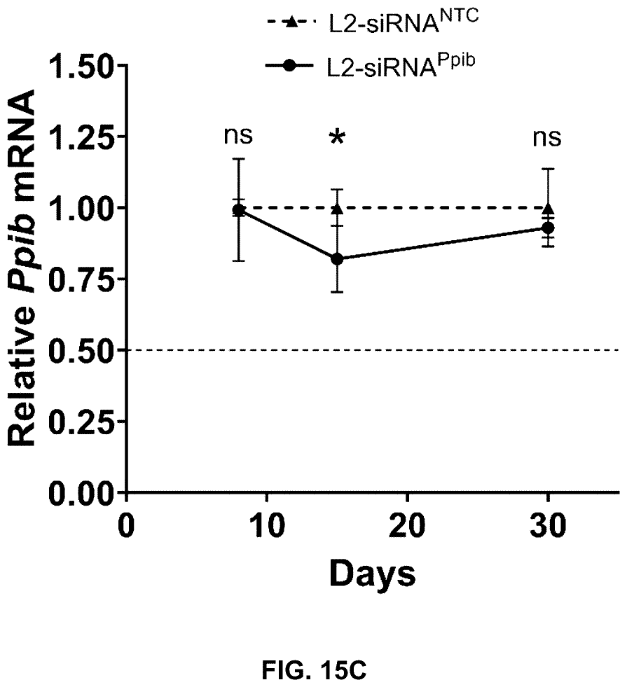

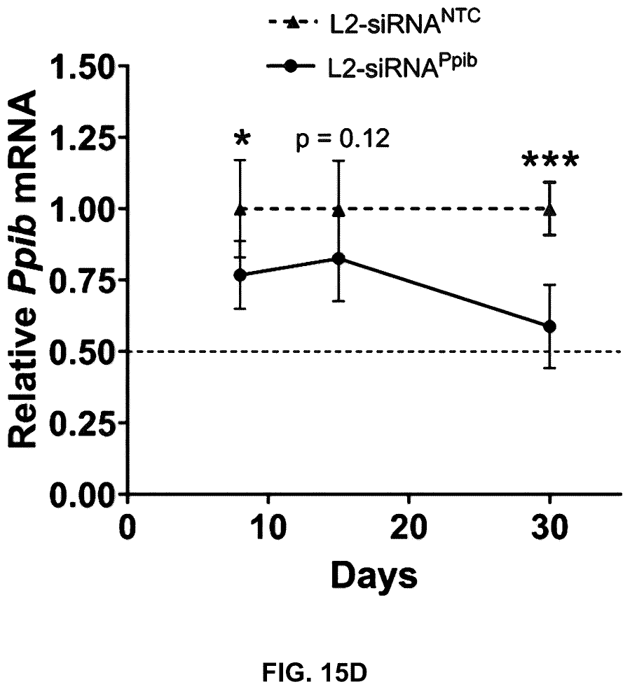

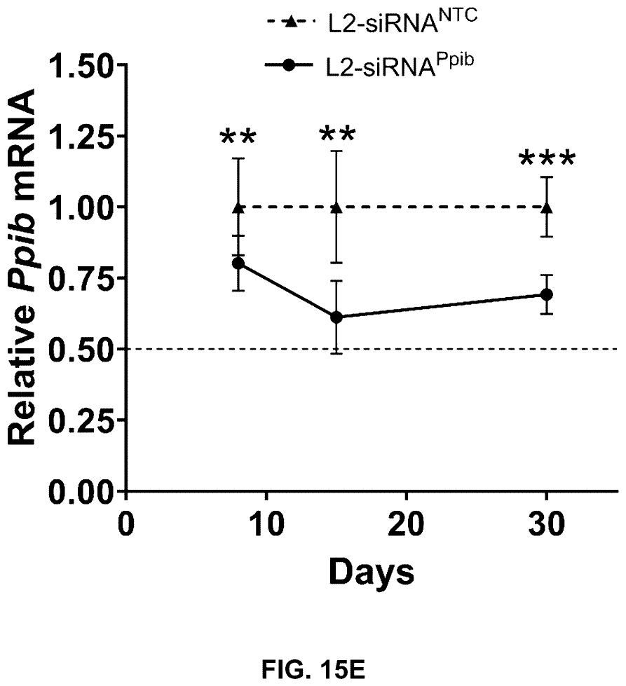

Based on the promising delivery of L2-siRNA to brain barrier cells, it was next sought to characterize potency and kinetics of target gene silencing. Ppib was chosen as a proof-of-concept target that is ubiquitously expressed by CNS cells and for which a well-validated siRNA sequence exists. Using the same dosing regime as biodistribution studies (20 mg/kg), knockdown at three timepoints (8, 15, 30 days) was assessed in CD31^{pos} MACS-sorted endothelial cells and manually dissected choroid plexuses (FIG. 15A). A non-targeting control (NTC) siRNA (termed L2-siRNA^{NTC}) (e.g., SEQ ID NO:7) was used as the primary negative control for gene silencing studies. It was found that L2-siRNA^{Ppib} potentiates robust silencing in brain endothelial cells at 8 and 15 days, and then returns to baseline expression by one month after a single injection (FIG. 15B). In contrast, CD31^{neg} cells, comprising all CNS cells except endothelial and choroid plexus cells, did not experience knockdown at 8 and 30 days, with a ˜20% decrease in Ppib at 15 days (FIG. 15C).

Knockdown was also measured in bulk choroid plexus residing in either the lateral or fourth ventricles. Interestingly, the lateral ventricle achieved peak knockdown 30 days after injection, highlighting a prolonged duration of gene silencing from a single intravenous injection (FIG. 15D). The fourth ventricle choroid plexus achieved peak knockdown at 15 days, but knockdown likewise persisted to 30 days, the longest time examined (FIG. 15E).

### Example 18

### Lipid-siRNA Conjugate Against CD33

To improve the translational relevance, a sequence against CD33 was developed, which is highly expressed by myeloid cells in the brain and classified as a high-quality AD therapeutic target. Further, by inhibiting gene expression of a new target, the versatility of the L2-siRNA platform is shown. Mechanistically, CD33 inhibits myeloid cell uptake and clearance of AB, and its genetic ablation from APP/PSEN1 and 5×FAD mice reduces plaque burden and improves cognition. From a therapeutic perspective, CD33 is considered “undruggable” with small molecules because the sialic acid ligand binding region is very flat, highly polar, and does not contain any binding pockets. Thus, genetic targeting is an appealing option for therapeutic intervention. As such, silencing CD33 expression for AD therapy with an siRNA-lipid conjugate is an innovative approach.

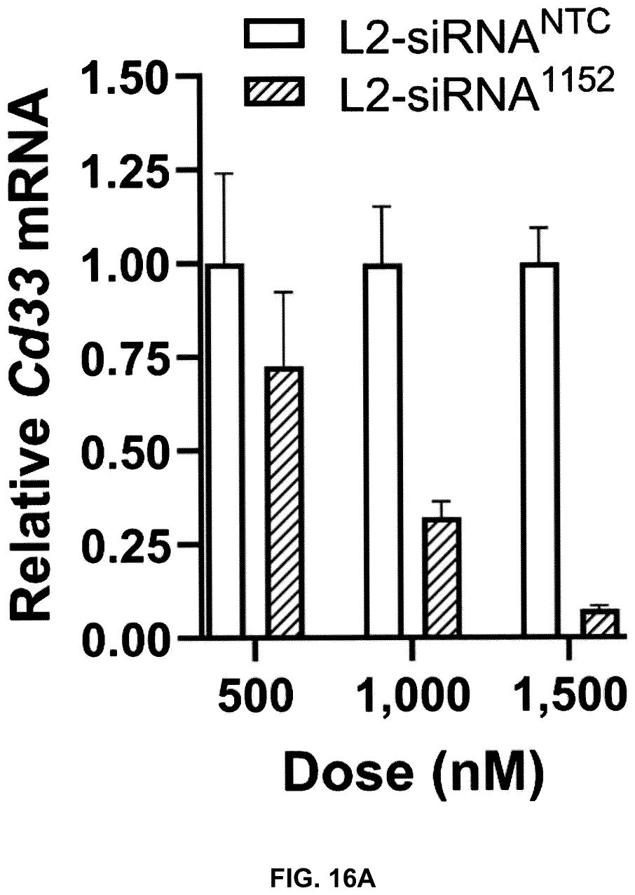

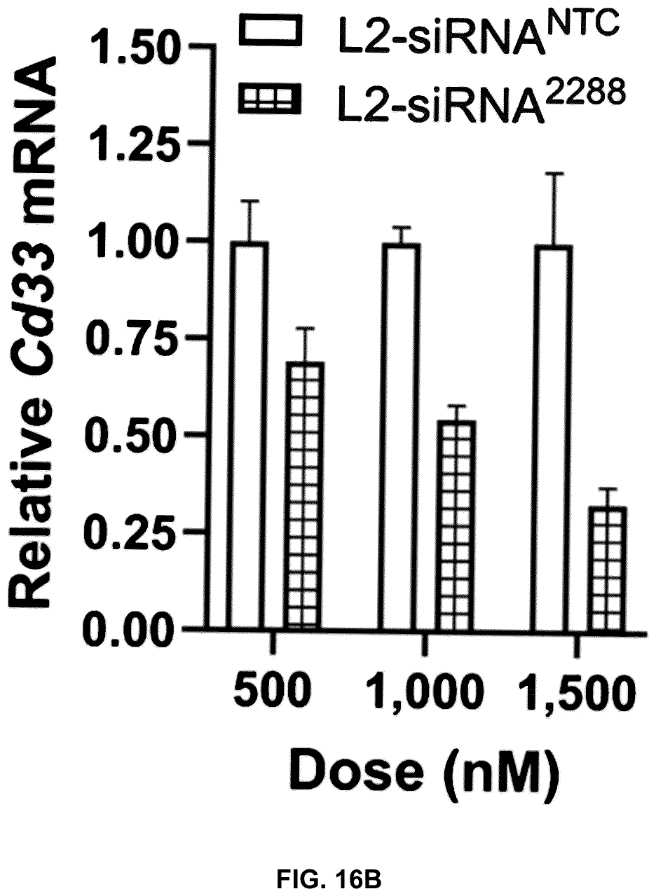

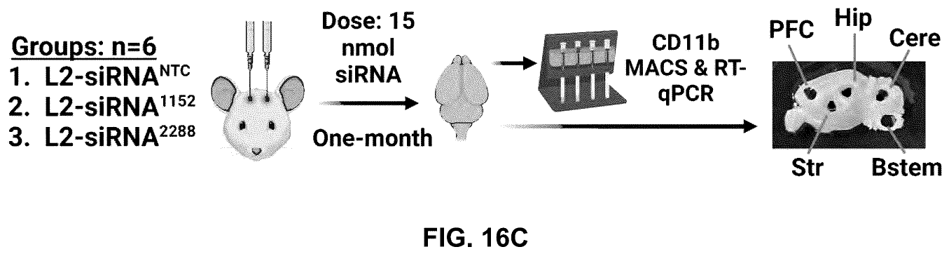

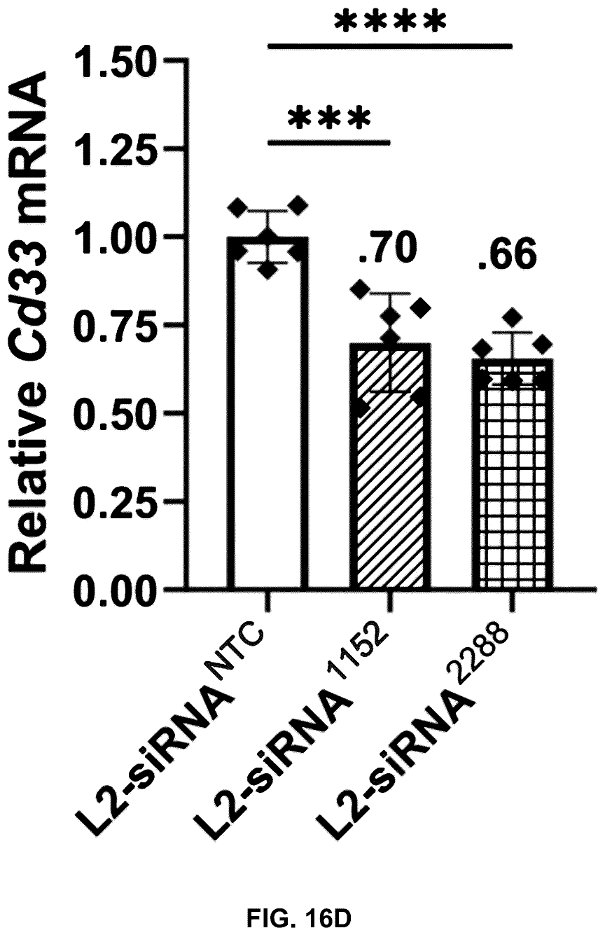

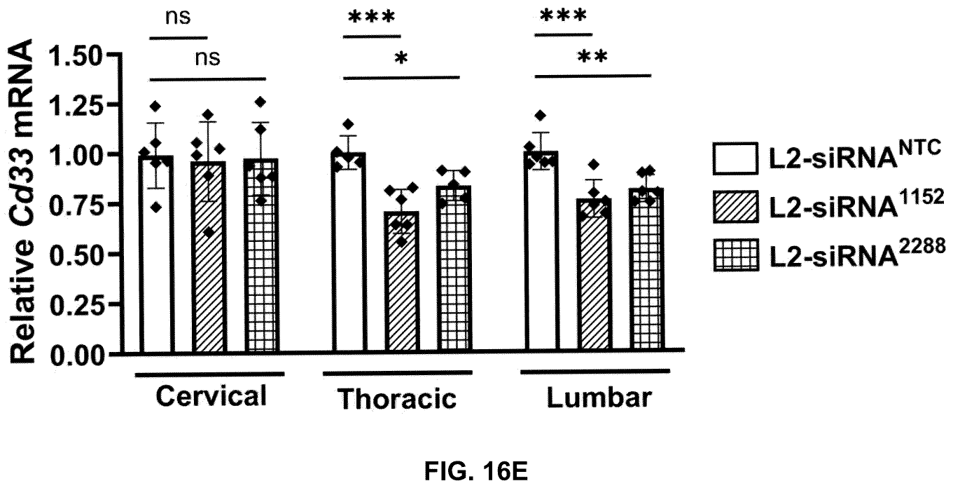

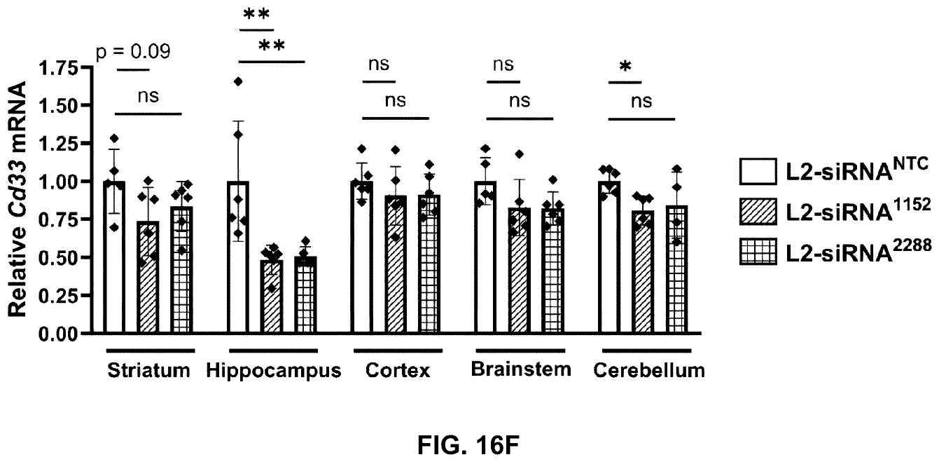

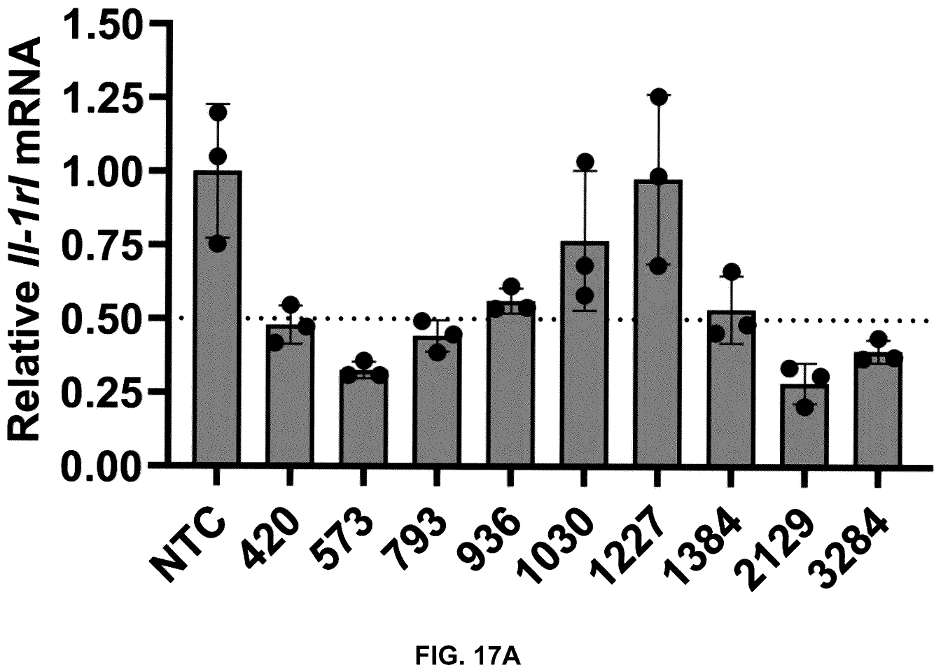

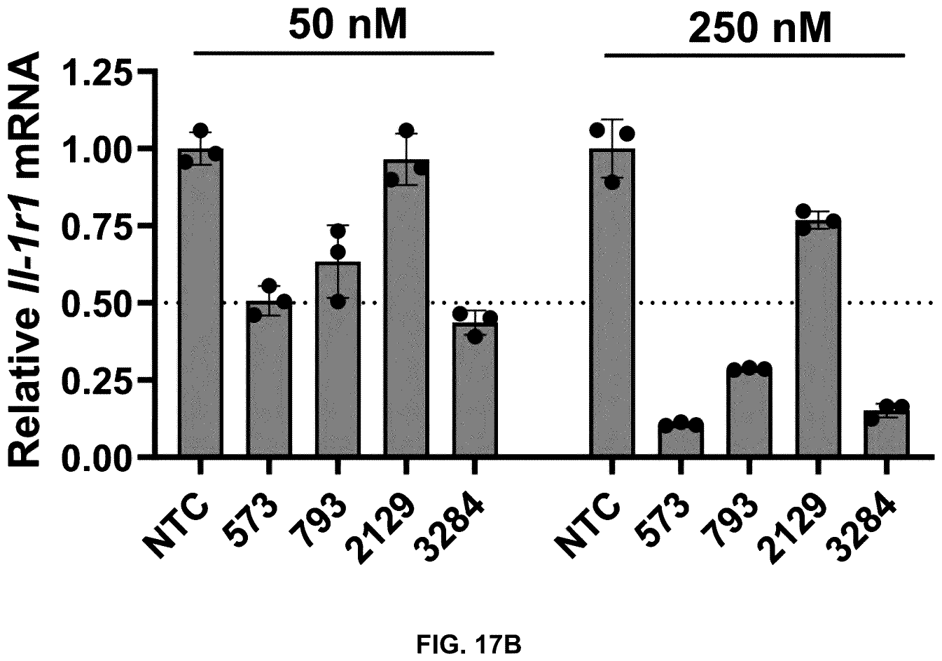

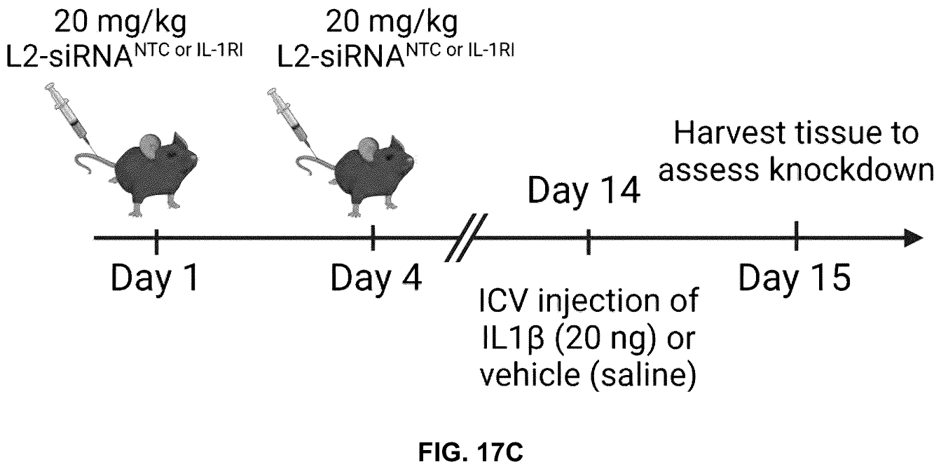

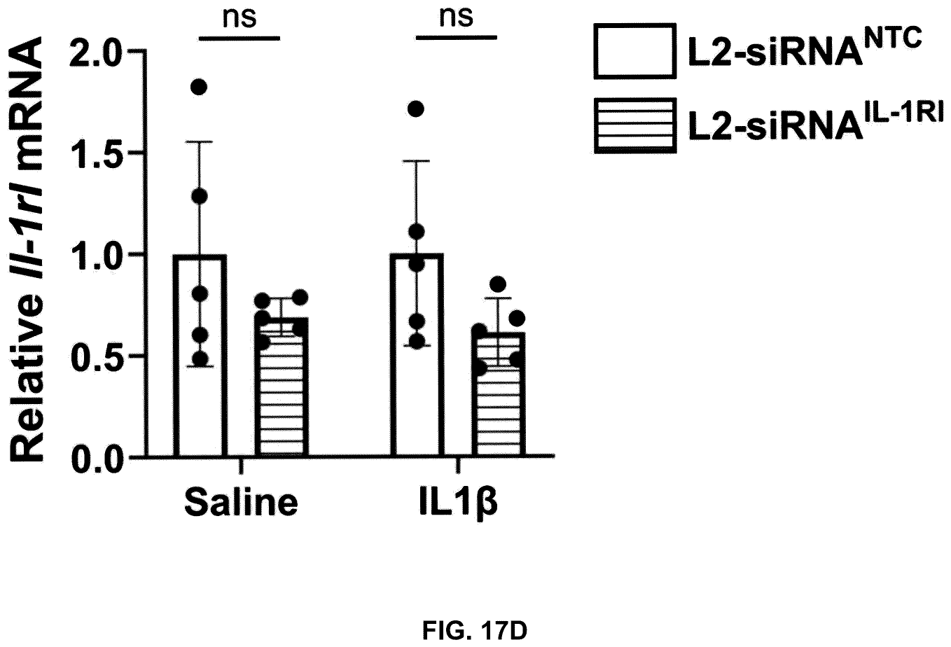

To identify a potent siRNA against CD33, sequences were designed and screened in vitro. Two lead candidates emerged (1152, 2288 named by mRNA position) and were synthesized with the L2 lipid for further characterization. Both sequences exhibited dose-dependent carrier-free gene silencing in RAW 264.7 macrophages after 48 hours (FIG. 16A and FIG. 16B). Having validated these new sequences in vitro, a study was conducted in mice to assess CNS gene silencing. Mice were injected ICV with L2-siRNA targeting CD33 with the 1152 sequence (L2-siRNA¹¹⁵²) or the 2288 sequence (L2-siRNA²²⁸⁸) and knockdown was compared to a non-targeting control L2-siRNA (L2-siRNA^{NTC}) (FIG. 16C). To evaluate knockdown in microglia, CD11b positive cells were isolated and RT-qPCR was performed. Robust gene knockdown of Cd33 was found with both sequences (FIG. 16D). Bulk tissue gene silencing was also examined in different regions of the brain and spinal cord, and knockdown was shown in the thoracic and lumbar regions of the spinal cord, as well as hippocampus and cerebellum (FIG. 16E and FIG. 16F). Additional data showing conjugates silencing IL-1R1 are shown in FIG. 17A, FIG. 17B, FIG. 17C, and FIG. 17D. In summary, it was shown that L2-siRNA is effective at silencing genes with new siRNA sequences, which is especially impressive in microglia, a cell type refractory to many gene therapy approaches.

It is understood that the foregoing detailed description and accompanying examples are merely illustrative and are not to be taken as limitations upon the scope of the disclosure.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the technology, may be made without departing from the spirit and scope thereof.

For reasons of completeness, various aspects of the disclosed technology are set out in the following numbered clauses:

Clause 1. A method of treating a central nervous system (CNS) disease in a subject in need thereof, the method comprising administering to the subject an effective amount of a conjugate, optionally in combination with a pharmaceutically acceptable excipient, wherein the conjugate comprises a siRNA capable of inhibiting expression of a protein associated with the CNS disease; a lipophilic ligand capable of binding albumin; and a linker attaching the siRNA to the lipophilic ligand, the linker comprising a branching molecule attached to the siRNA, and a hydrophilic spacer attaching the branching molecule to the lipophilic ligand.

Clause 2. The method of clause 1, wherein the conjugate is administered intravenously, intracerebroventricularly, intrathecally, subcutaneously, or intra-cisterna magna.

Clause 3. The method of clause 1 or 2, wherein the conjugate is administered intravenously.

Clause 4. The method of clause 1 or 2, wherein the conjugate is administered intracerebroventricularly.

Clause 5. The method of any one of clauses 1-4, wherein the CNS disease is Alzheimer's disease, Huntington's disease, a tauopathy, frontal temporal dementia, hydrocephalus, stroke, a brain tumor, or Amyotrophic Lateral Sclerosis.

Clause 6. The method of any one of clauses 1-5, wherein the CNS disease is Alzheimer's disease, Huntington's disease, or Amyotrophic Lateral Sclerosis.

Clause 7. The method of any one of clauses 1-6, wherein the conjugate is administered at a dosage of about 0.5 mg/kg to about 250 mg/kg.

Clause 8. The method of any one of clauses 1-7, wherein the method decreases the underlying pathology associated with the CNS disease in the subject for at least 30 days post-administration.

Clause 9. The method of any one of clauses 1-8, wherein the method inhibits the expression of the protein associated with the CNS disease in the subject's striatum, hippocampus, cortex, cerebellum, spinal cord, brain parenchyma, brain vasculature, choroid plexus, brain stem, or a combination thereof.

Clause 10. The method of any one of clauses 1-9, wherein the method inhibits the expression of the protein associated with the CNS disease for at least 3 months post-administration.

Clause 11. The method of any one of clauses 1-10, wherein the conjugate is administered once over 3 months.

Clause 12. The method of any one of clauses 1-11, wherein the pharmaceutically acceptable excipient comprises saline, phosphate buffered saline, albumin, dimethyl sulfoxide, trehalose, sucrose, polyethylene glycol, an absorption enhancer, or a combination thereof.

Clause 13. The method of any one of clauses 1-12, wherein the subject is human.

Clause 14. The method of any one of clauses 1-13, wherein the branching molecule includes at least one branch point having at least two independent branches.

Clause 15. The method of any one of clauses 1-14, wherein the hydrophilic spacer comprises 1 to 100 hydrophilic blocks.

Clause 16. The method of clause 15, wherein each hydrophilic block comprises 1 to 150 repeats of a hydrophilic compound.

Clause 17. The method of clause 16, wherein the hydrophilic compound comprises ethylene glycol, zwitterionic linkers, peptoids, amino acids, poly(glycerols), poly(oxazoline), poly(acrylamide), poly(N-acryloyl morpholine, poly(N,N-dimethyl acrylamide), poly(2-hydroxypropyl methacrylamide), poly(2-hydroxyethyl methacryalmide), or a combination thereof.

Clause 18. The method of any one of clauses 15-17, wherein each hydrophilic block comprises 1 to 100 repeats of ethylene glycol.

Clause 19. The method of any one of clauses 15-18, wherein the hydrophilic blocks are attached to each other through phosphorothioate linkages.

Clause 20. The method of any one of clauses 1-19, wherein the siRNA is capable of specifically hybridizing to an oligonucleotide encoding an amyloid protein, a tau protein, or an oncogene.

Clause 21. The method of any one of clauses 1-20, wherein the siRNA is capable of specifically hybridizing to an oligonucleotide encoding huntingtin, CD33, ApoE, MAPT, PDK1, C1QA, VCAM1, TREM2, SPP1, C3, SOD1, SERPINA3, or IL-1R1.

Clause 22. The method of any one of clauses 1-21, wherein the siRNA comprises a nucleotide sequence of SEQ ID NO: 1 to SEQ ID NO: 17, or a combination thereof.

Clause 23. The method of any one of clauses 1-22, wherein the siRNA comprises stabilizing modifications.

Clause 24. The method of any one of clauses 1-23, wherein the siRNA comprises a plurality of phosphorothioate linkages.

Clause 25. The method of any one of clauses 1-24, wherein the siRNA has about 15 nucleotides to about 40 nucleotides.

Clause 26. The method of any one of clauses 1-25, wherein the lipophilic ligand comprises a lipid including a C12-C22 hydrocarbon chain.

Clause 27. The method of any one of clauses 1-26, wherein the lipophilic ligand is divalent.

Clause 28. The method of any one of clauses 1-27, wherein the lipophilic ligand comprises two independent lipids, each lipid including a C12-C22 hydrocarbon chain.

Clause 29. The method of clause 28, wherein each lipid includes a C18 hydrocarbon chain.

Clause 30. The method of clause 26, wherein the lipid includes a carboxyl at its terminal end.

Clause 31. The method of clause 14, wherein each branch is attached to an individual hydrophilic spacer, and each hydrophilic spacer is attached to an individual lipid of the lipophilic ligand.

Clause 32. The method of any one of clauses 1-31, wherein the hydrophilic spacer is attached to the lipophilic ligand through a phosphorothioate linkage.

Clause 33. The method of any one of clauses 1-32, wherein the conjugate has a binding affinity (K_{d}) to albumin of less than 1 μM.

Clause 34. The method of any one of clauses 1-33, wherein the conjugate has a binding affinity (K_{d}) to albumin of less than 100 nM.

Clause 35. The method of any one of clauses 1-34, wherein the conjugate has a critical micelle concentration of greater than 1850 nM.

Clause 36. The method of any one of clauses 1-35, wherein the conjugate comprises about 20% to about 60% phosphorothioate linkages based on a total amount of phosphate-based linkages of the conjugate.

Clause 37. The method of any one of clauses 1-36, wherein the conjugate comprises a lipophilic ligand capable of binding albumin, the lipophilic ligand comprising two independent lipids, each lipid including a C18 hydrocarbon chain; and a linker attaching the siRNA to the lipophilic ligand, the linker comprising a branching molecule attached to the siRNA and including at least one branch point having at least two independent branches, and a hydrophilic spacer attaching an individual branch to an individual lipid, the hydrophilic spacer including 1 to 6 hydrophilic blocks, each hydrophilic block including 2 to 10 repeats of ethylene glycol.

Clause 38. A method of delivering a therapeutic to a central nervous system (CNS) of a subject in need thereof, the method comprising: administering a conjugate to the subject intravenously or intracerebroventricularly, wherein the conjugate localizes to the subject's CNS, and wherein the conjugate comprises a siRNA capable of inhibiting expression of a protein associated with the CNS disease; a lipophilic ligand capable of binding albumin; and a linker attaching the siRNA to the lipophilic ligand, the linker comprising a branching molecule attached to the siRNA, and a hydrophilic spacer attaching the branching molecule to the lipophilic ligand.

Clause 39. The method of clause 38, wherein the conjugate is administered intravenously and localizes to the subject's brain vasculature, choroid plexus, or both.

Clause 40. The method of clause 38, wherein the conjugate is administered intracerebroventricularly and localizes to the subject's perivascular region of the brain.

SEQUENCES

PPIB_AK (sense): 5′ UCACGAUGGAAUUUGCUGUU 3′ (SEQ ID NO: 1)
(see, e.g., Reynolds et al., Rational siRNA design for RNA
interference, Nature Biotechnology, 2004 Mar.; 22(3):326-30,
which is incorporated by reference herein in its entirety)
Htt (sense): 5′ UUAAUCUCUUUACUGAUAUA 3′ (SEQ ID NO: 2) (see,
e.g., U.S. Pat. No. 9,809,817, which is incorporated by reference
herein in its entirety)
Luc (sense): 5′ CAAUUGCACUGAUAAUGAA 3′ (SEQ ID NO: 3)
CD33_1152 (sense): 5′ ACUCAGAGAUCUAAAUGCA 3′ (SEQ ID NO: 4)
CD33_2288 (sense): 5′ GCACAUUCAUUAUCUCAUA 3′ (SEQ ID NO: 5)
IL-1R1_573 (sense): UCAGGAUAUUACUAUUGUA (SEQ ID NO: 6)
NTC (sense): UUCUCCGAACGUGUCACGU (SEQ ID NO: 7
IL-1R1 sequences: 5′→3′ sense strand. Lead sequence italicized.

| | |
|---|---|
| 420 | AUUCG CAAGU GUCCU CUUA (SEQ ID NO: 8) |
| 573 | UCAGG AUAUU ACUAU UGUA (SEQ ID NO: 9) |
| 715 | GAAGU CUUGU GUGCC CUUA (SEQ ID NO: 10) |
| 793 | GUAAA CCUCU GCUUC UUGA (SEQ ID NO: 11) |
| 936 | GUAAU ACAAU UUAUC ACAA (SEQ ID NO: 12) |
| 1030 | UGAUA CAACU GAUCU GCAA (SEQ ID NO: 13) |
| 1227 | CCGUU UAUCU GUGUU GUUA (SEQ ID NO: 14) |
| 1384 | UUGAC AUAGU GCUUU GGUA (SEQ ID NO: 15) |
| 2129 | AGUCC CUUGU UCCAG AUCA (SEQ ID NO: 16) |
| 3284 | GUAUU UAAAU UUGCA AUUA (SEQ ID NO: 17) |

What is claimed is:

1. A method of treating a central nervous system (CNS) disease in a subject in need thereof, the method comprising administering to the subject an effective amount of a conjugate, optionally in combination with a pharmaceutically acceptable excipient, wherein the conjugate comprises
a siRNA capable of inhibiting expression of a protein associated with the CNS disease;
a lipophilic ligand capable of binding albumin; and
a linker attaching the siRNA to the lipophilic ligand, the linker comprising
a branching molecule attached to the siRNA, and
a hydrophilic spacer attaching the branching molecule to the lipophilic ligand.

2. The method of claim 1, wherein the conjugate is administered intravenously, intracerebroventricularly, intrathecally, subcutaneously, or intra-cisterna magna.

3. The method of claim 1, wherein the conjugate is administered intravenously.

4. The method of claim 1, wherein the conjugate is administered intracerebroventricularly.

5. The method of claim 1, wherein the CNS disease is Alzheimer's disease, Huntington's disease, a tauopathy, frontal temporal dementia, hydrocephalus, stroke, a brain tumor, or Amyotrophic Lateral Sclerosis.

6. The method of claim 5, wherein the CNS disease is Alzheimer's disease, Huntington's disease, or Amyotrophic Lateral Sclerosis.

7. The method of claim 1, wherein the conjugate is administered at a dosage of about 0.5 mg/kg to about 250 mg/kg.

8. The method of claim 1, wherein the method decreases the underlying pathology associated with the CNS disease in the subject for at least 30 days post-administration.

9. The method of claim 1, wherein the method inhibits the expression of the protein associated with the CNS disease in the subject's striatum, hippocampus, cortex, cerebellum, spinal cord, brain parenchyma, brain vasculature, choroid plexus, brain stem, or a combination thereof.

10. The method of claim 1, wherein the method inhibits the expression of the protein associated with the CNS disease for at least 3 months post-administration.

11. The method of claim 10, wherein the conjugate is administered once over 3 months.

12. The method of claim 1, wherein the pharmaceutically acceptable excipient comprises saline, phosphate buffered saline, albumin, dimethyl sulfoxide, trehalose, sucrose, polyethylene glycol, an absorption enhancer, or a combination thereof.

13. The method of claim 1, wherein the subject is human.

14. The method of claim 1, wherein the branching molecule includes at least one branch point having at least two independent branches.

15. The method of claim 1, wherein the hydrophilic spacer comprises 1 to 100 hydrophilic blocks.

16. The method of claim 15, wherein each hydrophilic block comprises 1 to 150 repeats of a hydrophilic compound.

17. The method of claim 16, wherein the hydrophilic compound comprises ethylene glycol, zwitterionic linkers, peptoids, amino acids, poly(glycerols), poly(oxazoline), poly(acrylamide), poly(N-acryloyl morpholine, poly(N,N-dimethyl acrylamide), poly(2-hydroxypropyl methacrylamide), poly(2-hydroxyethyl methacryalmide), or a combination thereof.

18. The method of claim 16, wherein each hydrophilic block comprises 1 to 100 repeats of ethylene glycol.

19. The method of claim 16, wherein the hydrophilic blocks are attached to each other through phosphorothioate linkages.

20. The method of claim 1, wherein the siRNA is capable of specifically hybridizing to an oligonucleotide encoding an amyloid protein, a tau protein, or an oncogene.

21. The method of claim 1, wherein the siRNA is capable of specifically hybridizing to an oligonucleotide encoding huntingtin, CD33, ApoE, MAPT, PDK1, C1QA, VCAM1, TREM2, SPP1, C3, SOD1, SERPINA3, or IL-1R1.

22. The method of claim 1, wherein the siRNA comprises a nucleotide sequence of SEQ ID NO: 1 to SEQ ID NO: 17, or a combination thereof.

23. The method of claim 1, wherein the siRNA comprises stabilizing modifications.

24. The method of claim 1, wherein the siRNA comprises a plurality of phosphorothioate linkages.

25. The method of claim 1, wherein the siRNA has about 15 nucleotides to about 40 nucleotides in length.

26. The method of claim 1, wherein the lipophilic ligand comprises a lipid including a C12-C22 hydrocarbon chain.

27. The method of claim 1, wherein the lipophilic ligand is divalent.

28. The method of claim 1, wherein the lipophilic ligand comprises two independent lipids, each lipid including a C12-C22 hydrocarbon chain.

29. The method of claim 28, wherein each lipid includes a C18 hydrocarbon chain.

30. The method of claim 26, wherein the lipid includes a carboxyl at its terminal end.

31. The method of claim 14, wherein each branch is attached to an individual hydrophilic spacer, and each hydrophilic spacer is attached to an individual lipid of the lipophilic ligand.

32. The method of claim 1, wherein the hydrophilic spacer is attached to the lipophilic ligand through a phosphorothioate linkage.

33. The method of claim 1, wherein the conjugate has a binding affinity (K_{d}) to albumin of less than 1 μM.

34. The method of claim 1, wherein the conjugate has a binding affinity (K_{d}) to albumin of less than 100 nM.

35. The method of claim 1, wherein the conjugate has a critical micelle concentration of greater than 1850 nM.

36. The method of claim 1, wherein the conjugate comprises about 20% to about 60% phosphorothioate linkages based on a total amount of phosphate-based linkages of the conjugate.

37. The method of claim 1, wherein the conjugate comprises
a lipophilic ligand capable of binding albumin, the lipophilic ligand comprising two independent lipids, each lipid including a C18 hydrocarbon chain; and
a linker attaching the siRNA to the lipophilic ligand, the linker comprising
a branching molecule attached to the siRNA and including at least one branch point having at least two independent branches, and
a hydrophilic spacer attaching an individual branch to an individual lipid, the hydrophilic spacer including 1 to 6 hydrophilic blocks, each hydrophilic block including 2 to 10 repeats of ethylene glycol.

38. A method of delivering a therapeutic to a central nervous system (CNS) of a subject in need thereof, the method comprising:
administering a conjugate to the subject intravenously or intracerebroventricularly, wherein the conjugate localizes to the subject's CNS, and wherein the conjugate comprises
a siRNA capable of inhibiting expression of a protein associated with the CNS disease;
a lipophilic ligand capable of binding albumin; and
a linker attaching the siRNA to the lipophilic ligand, the linker comprising
a branching molecule attached to the siRNA, and
a hydrophilic spacer attaching the branching molecule to the lipophilic ligand.

39. The method of claim 38, wherein the conjugate is administered intravenously and localizes to the subject's brain vasculature, choroid plexus, or both.

40. The method of claim 38, wherein the conjugate is administered intracerebroventricularly and localizes to the subject's perivascular region of the brain.


SEQUENCE LISTING (17 sequences)

SEQ ID NO: 1 - RNA, 20 nt, synthetic construct
  source 1..20: mol_type = other RNA
tcacgatggaatttgctgtt

SEQ ID NO: 2 - RNA, 20 nt, synthetic construct
  source 1..20: mol_type = other RNA
ttaatctctttactgatata

SEQ ID NO: 3 - RNA, 19 nt, synthetic construct
  source 1..19: mol_type = other RNA
caattgcactgataatgaa

SEQ ID NO: 4 - RNA, 19 nt, synthetic construct
  source 1..19: mol_type = other RNA
actcagagatctaaatgca

SEQ ID NO: 5 - RNA, 19 nt, synthetic construct
  source 1..19: mol_type = other RNA
gcacattcattatctcata

SEQ ID NO: 6 - RNA, 19 nt, synthetic construct
  source 1..19: mol_type = other RNA
tcaggatattactattgta

SEQ ID NO: 7 - RNA, 19 nt, synthetic construct
  source 1..19: mol_type = other RNA
ttctccgaacgtgtcacgt

SEQ ID NO: 8 - RNA, 19 nt, synthetic construct
  source 1..19: mol_type = other RNA
attcgcaagtgtcctctta

SEQ ID NO: 9 - RNA, 19 nt, synthetic construct
  source 1..19: mol_type = other RNA
tcaggatattactattgta

SEQ ID NO: 10 - RNA, 19 nt, synthetic construct
  source 1..19: mol_type = other RNA
gaagtcttgtgtgccctta

SEQ ID NO: 11 - RNA, 19 nt, synthetic construct
  source 1..19: mol_type = other RNA
gtaaacctctgcttcttga

SEQ ID NO: 12 - RNA, 19 nt, synthetic construct
  source 1..19: mol_type = other RNA
gtaatacaatttatcacaa

SEQ ID NO: 13 - RNA, 19 nt, synthetic construct
  source 1..19: mol_type = other RNA
tgatacaactgatctgcaa

SEQ ID NO: 14 - RNA, 19 nt, synthetic construct
  source 1..19: mol_type = other RNA
ccgtttatctgtgttgtta

SEQ ID NO: 15 - RNA, 19 nt, synthetic construct
  source 1..19: mol_type = other RNA
ttgacatagtgctttggta

SEQ ID NO: 16 - RNA, 19 nt, synthetic construct
  source 1..19: mol_type = other RNA
agtcccttgttccagatca

SEQ ID NO: 17 - RNA, 19 nt, synthetic construct
  source 1..19: mol_type = other RNA
gtatttaaatttgcaatta